(19) United States
(12) Patent Application Publication
(10) Pub. No.: US 2016/0215348 A1
(43) Pub. Date: Jul. 28, 2016

(54) GENE EXPRESSION BIOMARKERS AND THEIR USE FOR DIAGNOSTIC AND PROGNOSTIC APPLICATION IN PATIENTS POTENTIALLY IN NEED OF HDAC INHIBITOR TREATMENT

(71) Applicant: 4SC AG, Planegg-Martinsried (DE)

(72) Inventors: Hella KOHLHOF, Munchen (DE); Thomas HERZ, Stockdorf (DE); Robert DOBLHOFER, Hohenkirchen-Siegertsbrunn (DE); Thomas BECKERS (Deceased), Konstanz (DE); Astrid ZIMMERMANN, Muhltal (DE); Marina MOLLENHAUER-THEIN, Bremen (DE); Markus BOEHM, Rheinfelden (DE); Volker GEKELER, Konstanz (DE); Hans-Peter HOFMANN, Otterfing (DE); Thomas MAIER, Stockach (DE); Eike STAUB, Darmstadt (DE); Timo WITTENBERGER, Konstanz (DE); Martin ELMLINGER, Kreuzlingen (CH)

(73) Assignee: 4SC AG, Planegg-Martinsried (DE)

(21) Appl. No.: 14/892,625

(22) PCT Filed: May 22, 2014
(86) PCT No.: PCT/EP2014/060532

§ 371 Date: Nov. 20, 2015

Related U.S. Application Data
(60) Provisional application No. 61/827,201, filed on May 24, 2013.
(60) Provisional application No. 61/865,712, filed on Aug. 14, 2013.
(60) Provisional application No. 61/935,914, filed on Feb. 5, 2014.

(51) Int. Cl. C12Q 1/68; G01N 33/574

(52) U.S. Cl. CPC C12Q 1/6886; G01N 33/57484; C12Q 2600/158; C12Q 2600/106

(57) ABSTRACT

The present invention relates to the utilization of one or more genes selected from the group comprising ZFP64, DPP3, CCDC43, HIST2H4A/B, KDELC2 and MICALL1 as biomarkers for HDAC inhibitor treatment. The expression and/or change of the aforementioned genes are preferably determined via the respective corresponding mRNA or one or more proteins expressed by the aforementioned genes.

The present invention is directed to certain specific biomarkers which may be used in connection to HDAC inhibitor treatment, methods wherein said biomarkers are applied, and kits for use in said methods.

### BACKGROUND OF THE INVENTION

The study of heritable changes in phenotype (appearance) or gene expression caused by mechanisms other than changes in the underlying DNA sequence is called epigenetics. It is possible that such changes remain through cell divisions for the remainder of the cell's life and may exist for multiple generations. Said non-genetic factors cause the organism's genes to behave or express themselves differently (Special report: “What genes remember” by Philip Hunter, Prospect Magazine May 2008 issue 146).

Recent epigenetics research has shown that environmental factors influence characteristics of organisms and may sometimes be passed on to the offspring. By today it has been scientifically proven, which molecular structures are involved: important factors are structural components of the chromosomes, the histones, a sort of packaging material for the DNA, in order to store DNA in an ordered and space-saving way. Depending on the chemical groups and corresponding modifications they carry, e.g., if they are acetylated, phosphorylated or methylated, they permanently activate or deactivate genes.

Taken together, more than 100 examples of transgenerational epigenetic inheritance phenomena have been reported in a wide range of organisms, including prokaryotes, plants, and animals (Jablonka, Eva; Gal Raz, 2009, The Quarterly Review of Biology 84 (2): 131-176).

The molecular basis of epigenetic mechanisms is complex and heterogeneous and involves modifications of the activation status of certain genes, but not the basic structure and sequence of DNA. Furthermore, the chromatin proteins associated with the DNA may be in an activated or silenced state induced by a variety of protein modifications. As mentioned above, also such epigenetic changes, e.g., to the chromatin are preserved when cells divide. Based on these modifications the way the DNA is wrapped around the histones is changed, and due to this structural modification, gene expression is changed as well. Such mechanisms of chromatin remodeling may be accomplished through several mechanisms.

One important process comprises post translational modifications of the amino acids that make up histone proteins, which may occur e.g. as acetylation, methylation and/or phosphorylation. If the amino acids are modified, the overall shape of the histone protein might be changed. Also, DNA is not completely unwound during replication, and therefore, it appears possible that such modified histones may be carried into each new copy of the DNA. These modified histones may then act as template structures, having the surrounding new histones also shaped in a modified manner.

The unstructured N-termini of histones (also called “histone tail”) are particularly highly modified, but histone modifications may occur throughout the entire sequence. These modifications include acetylation, methylation, ubiquitinylation, phosphorylation and sumoylation. For example, acetylation of the K14 and K9 lysines of the tail of histone H3 by histone acetyltransferase enzymes (HATs) is generally correlated with transcriptional competence. Deacetylation accordingly is correlated with transcriptional silencing and is being served by enzymes having histone deacetylase (HDAC) activity.

It is thought that the tendency of acetylation to be associated with “active” transcription is biophysical in nature. Because a lysine residue normally has a positively charged nitrogen at its end, and can therefore associate to the negatively charged phosphates of the DNA backbone. In contrast, once an acetylation event changes this positively charged amine group on the lysine side chain, it is converted into a neutral amide linkage, resulting in loosening the DNA from the histone. When this occurs, transcriptional factors and complexes can bind more easily to the DNA and allow transcriptional processes to occur. This may be referred to as the “cis” model of epigenetic mechanisms in which changes to the histone tails have a direct effect on the DNA itself. In another model of epigenetic mechanisms, the “trans” model, changes to the histone tails act indirectly on the DNA. For example, a lysine acetylation may create a binding site for chromatin modifying enzymes (and the basal transcription machinery as well) which then cause changes to the state of the chromatin. Indeed, the conserved bromodomain, a protein segment (domain) that specifically binds acetyl-lysine, is found in many enzymes that help activate transcription, including the SWI/SNF complex (on the protein polybromo). In summary, it appears that acetylation acts in both “cis” and “trans” models to modify transcriptional activation.

Different histone modifications are believed to function in different ways; acetylation at one position is likely to function differently than acetylation at another position. Also, multiple modifications may occur, and these modifications may work together to change the behavior of the nucleosome structure (DNA plus histones). These underlying multiple dynamic modifications of histones regulate gene transcription in a systematic and reproducible way and are referred to as the histone code.

Modulating epigenetics mechanisms holds promise for a variety of potential medical applications. Congenital genetic disease is well understood, but it is also clear that epigenetics is important, as e.g., in the case of Angelman syndrome and Prader-Willi syndrome. These are normal genetic diseases caused by gene deletions or inactivation of the genes, but are unusually common because affected individuals are essentially hemizygous because of genomic imprinting, and therefore a single gene knock out is sufficient to cause the disease, where most cases would require both copies to be knocked out (Online ‘Mendelian Inheritance in Man’, OMIM, www.ncbi.nlm.nih.gov/omim).

Even though epigenetic mechanisms in multicellular organisms have generally thought to be involved in differentiation, there have even been some observations of transgenerational epigenetic inheritance in various species. Most of these multigenerational epigenetic traits may be gradually lost over several generations, but the possibility remains that multigenerational epigenetics could add another aspect to evolution and adaptation. The modification of epigenetic features associated with a region of DNA allows organisms, on a multigenerational time scale, to switch between phenotypes that express and repress that particular gene (O. J. Rando and K. J. Verstrepen, 2007, Cell 128 (4): 655-668). When the DNA sequence of the region is not mutated, this change is reversible and offers flexibility for adaptive processes.

Current research has shown that epigenetic pharmaceuticals could be a putative replacement or adjuvant therapy for currently accepted treatment methods such as radiation and chemotherapy, or could enhance the effects of these current treatments (Wang, L G; Chiao, J W, 2010, Int. J. Oncolo. 3 (37): 533-9). It was shown that the epigenetic control of, e.g., proto-oncogene regions and of tumor suppressor sequences by conformational changes in histones directly affects the formation and progression of cancer (Iglesias-Linares et al., 2010, Oral Oncology 5 (46): 323-9).

Such new treatment options with drugs that act epigenetically could furthermore offer the opportunity of reversibility, a characteristic that other cancer treatments do not offer (Li, L C; Carroll, P R; Dahiya, R, 2005. JNCI 2 (97): 103-15). By today, epigenetic drug development has mainly focused on histone acetyltransferases (HAT) and histone deactylases (HDAC), including the introduction of the new HDAC inhibitory pharmaceuticals Vorinostat and Romidepsin to the market (Spannhoff, A; Sippl, W; Jung, M (2009), International Journal of Biochemistry & Cell Biology 1 (41): 4-11). HDAC enzymes specifically have been shown to play an integral role in the progression of oral squamous cancer (Iglesias-Linares et al., (2010), Oral Oncology 5 (46): 323-9). Overexpression of selected HDAC isoenzymes has recently been linked to a worsening prognosis in different cancer types, such as HDAC-1 and HDAC-2 in hepatocellular cancer and others (Lee T K, Poh Y P et al., 2011: The Journal of Clinical Investigation, published online February 2011, www.jci.org), or HDAC-2 in colorectal tumors (Zhu P, Martin E, Mengwasser J, Schlag P, Janssen K P, Göttlicher M., 2004, Cancer Cell. 2004 May; 5(5):455-63). As described above, aberrant HDAC enzyme expression or activity was found to be associated with a number of human malignancies causing repression of well-known tumor-suppressor genes and modifying the activity of other factors important for malignant progression. Thus, inhibition of histone deacetylases represents a promising therapeutic concept in oncology drug development.

Today it is in fact well accepted that not only the genetic makeup, i.e., the direct base sequence of the DNA, and/or mutations of genes of an individual, contributes to the pathogenesis of various diseases, notably cancer, but furthermore, also the influence of, e.g., environmental signals which influence the epigenetic secondary packaging into chromation may influence or cause pathogenic events, contributing to cancerogenesis.

The aforementioned epigenetic changes may even be passed forward from one cell generation to another, or may even be passed on to the offspring in general, thus, providing an individual with both, a genetic and also an epigenetic makeup.

Therefore, it has to be noted, that not only the genetic makeup, or mutations in the genes, but also the overall epigenetic makeup of an individual may contribute to the development of a disease and may contribute to the body's response to drugs that influence such epigenetic mechanisms. This way, the observation of changes in the epigenetic makeup of an individual in any cell of the body induced by drugs that impact on epigenetic mechanisms may represent a valid method to predict an individual's response to treatment. The diseased tissue of such an individual is likely to respond to the drug having an effect on an epigenetic level in the same or at least similar way as a non-diseased tissue in which such a drug effect may be measured.

Such drugs are for example represented by inhibitors of enzymes having histone deacetylase activity, or rather today referred to as protein deacetylases in general, since their deacetylation activity is frequently not limited to histones as client proteins, and may impact more broadly directly or indirectly on proto-oncogene and tumor suppressor protein function.

Various HDAC inhibitors are currently under clinical investigation in a broad range of tumor entities including both, hematologic malignancies and solid tumors, and represent a class of epigenetically active, potent anti-proliferative, differentiation-inducing and pro-apoptotic agents. Two members of the histone deacetylase inhibitor family (Vorinostat and Romidepsin) have already been approved for treatment of refractory cutaneous T-cell lymphoma, showing considerable clinical benefit as mono therapeutic agent in these patients.

A number of further HDAC inhibitors are currently in clinical development at various stages, including panobinostat, entinostat, belinostat, givinostat and resminostat (Marks, P A and Wu, W-s, 2009, J. Cell. Biochem.; 107(4): 600-608) (Ellis, L and Pili, R, 2010, Pharmaceuticals (Basel); 3(8); 2411-2469).

However, there is a need in the art for determining the effect of an HDAC inhibitor treatment as early as possible in the intent or process of treatment.

ZFP64, a transcription factor of the C2H2-type zinc finger protein (ZFP) family plays an important role in many cell functions including development, differentiation, tumorigenesis and immune response. It is a positive regulator in TLR signaling with NF-κB activation and subsequent inflammatory response to invading pathogens (Wang et al., J Biol Chem 2013, published online Jul. 15, 2013). However, its biological function remains largely unknown.

Based on IHC staining experiments (see http://www.proteinatlas.org/ENSG00000020256/tissue/staining+overview) ZFP64 protein is expressed preferably in certain tissues/organs and is predominantly found in the nucleus. ZFP64 protein in normal tissue/organs is preferably expressed in: Liver, Pancreas, and GI tract, as well as Testis, and Skin. ZFP64 protein in cancer tissue is preferably expressed in the following cancer types: Liver, Lymphoma, Pancreas, Thyroid and Renal. ZFP64 is up-regulated in liver metastases compared to the primary tumor in CRC patients (Li et al., Hepatobiliary Pancreat Dis Int. 2010; 9:149-53).

ZFP64 has been identified to regulate differentiation of mesenchymal cells by co-activation of Notch1. ZFP64 is reported to be associated with the intracellular domain of Notch1 (NICD), and is recruited to the promoters of the Notch target genes Hesl and Heyl, and transactivates them, and is involved in the differentiation of mesenchymal cells by co-activation of Notch1 (Sakamoto et al., J Cell Sci. 2008 May 15; 121(Pt 10):1613-23. doi: 10.1242/jcs.023119).

### BRIEF SUMMARY OF THE INVENTION

In preclinical and clinical studies it was identified that the use of HDAC inhibitors resulted in the reproducible modulation of specific gene expression patterns. It could be shown that HDAC inhibitors regulated those gene expressions in the same way in both, (i) in a variety of different cancer cell types treated with the HDAC inhibitors in vitro, as well as (ii) in peripheral blood cells of cancer patients treated with the HDAC inhibitors in the context of clinical studies and (iii) in PBMCs of healthy donors treated with HDAC inhibitors ex vivo.

It can therefore be reasoned that the overall epigenetic makeup of cancer cells and peripheral blood cells of a given patient is comparable with respect to its susceptibility to the influence of certain pharmacological agents, such as HDAC inhibitors, and that therefore both cell types will experience the same or similar gene expression modulations. Therefore, the gene expression changes in certain specific genes induced epigenetically in patients' peripheral blood cells can potentially be used to determine the drug's activity on the same patients' tumor cells.

Furthermore, a decrease or increase of these epigenetically induced changes in gene expression profiles during treatment of a patient with an HDAC inhibitor may indicate a decreased or increased therapeutic benefit for the patient and may therefore correlate with a disease prognosis.

### BRIEF DESCRIPTION OF THE PRESENT INVENTION

In one aspect, the present invention relates to the modulation of gene expression of specific selected genes which are reproducibly changed upon exposure to an HDAC inhibitor, in cancer cell lines and in PBMCs, as well as in peripheral blood cells of cancer patients.

It is expected that these gene expression modulations, also herein referred to as biomarkers, have various utilities.

They may serve as markers of the pharmacodynamic activity of the HDAC inhibitor applied.

They may serve as predictive biomarkers (or for stratification) prior to engagement into treatment with an HDAC inhibitor allowing for a prediction whether a patient should be treated with an HDAC inhibitor and whether in a patient to be treated with an HDAC inhibitor a clinical benefit may be expected or not.

They may serve as prognostic biomarkers prior to engagement into treatment with an HDAC inhibitor allowing for a prediction how the disease will progress for a given patient, independent of treatment.

They may serve as biomarkers during treatment with an HDAC inhibitor in order to predict how long a patient may benefit from the treatment with the HDAC inhibitor, and in general to monitor progress of the HDAC inhibitor treatment.

Furthermore, these biomarkers may also be causally involved in the disease progress and may therefore represent therapeutic target structures on their own. Therefore, their activity may be subject to therapeutic interference utilizing various means, such as influencing their mRNA expression pattern by, e.g., siRNA interference, via gene therapy to increase their presence, antibody technology to modulate their protein functions, or small molecule binders which change the functional potency of such biomarker entities. Also vaccination processes could be possible.

Therefore, subject matter of the present invention is the use of a at least one gene, a DNA sequence of said at least one gene, an RNA sequence encoded by said at least one gene or fragments thereof of at least 150, preferably 180 nucleotides in length, or at least one protein encoded by said at least one gene, or a domain of said protein, in diagnostic and prognostic methods related to HDAC inhibitor treatment and for monitoring an HDAC inhibitor treatment or for stratifying patients, wherein said at least one gene is selected from one or more members the group comprising ZFP64, DPP3, CCDC43, HIST2H4A/B, KDELC2 and MICALL1. A further embodiment of the present invention is the use of the aforementioned at least one gene, DNA sequence, RNA sequence or protein, respectively, as targets for therapeutic interference.

The present invention encompasses the application of the biomarkers, nucleotide sequences, proteins, kits, methods and uses according to the present invention for monitoring HDAC inhibitor treatment and for stratifying patients potentially in need of said treatment into responders or non-responders.

The identification of the genes selected for this invention is given in table 1.

TABLE 1
Biomarkers of the present invention, identified by NCBI symbol,
gene name and Entrez ID

| Official | | |
|---|---|---|
| Symbol | | |
| (NCBI | | Entrez Gene ID |
| Database) | Gene Name [human] | [human] |
| CCDC43 | coiled-coil domain containing 43 | 124808 |
| DPP3 | dipeptidyl-peptidase 3 | 10072 |
| HIST2H4A | histone cluster 2, H4a and histone | 8370 and 554313 |
| and | cluster 2, H4b | |
| HIST2H4B | | |
| KDELC2 | KDEL (Lys-Asp-Glu-Leu) containing 2 | 143888 |
| MICALL1 | MICAL-like 1 | 85377 |
| ZFP64 | zinc finger protein 64 homolog (mouse) | 55734 |

### DETAILED DESCRIPTION OF THE EMBODIMENTS

One subject of the present invention is to measure the gene expression of one or more of the genes according to the present invention, either directly in a sample of a diseased tissue or in peripheral blood cells, either prior to start of the treatment with an HDAC inhibitor or during the course of the treatment. Furthermore, another subject of the invention is to measure the change in these expression profiles of one or more of the genes selected in this invention, comparing the gene expressions before start of the treatment with the gene expressions observed during treatment. Furthermore, another subject of the invention is to measure the difference in these expression profiles of one or more of the genes selected in this invention, comparing the gene expressions between different subgroups of the patients receiving treatment.

[symbol]

Certain embodiments of the present invention are listed in the following.
1. A method of determining an effect of an HDAC inhibitor treatment, the method comprising the following steps:
   a) Providing a sample of a patient receiving said HDAC inhibitor treatment,
   b) determining the gene expression and/or the change of the gene expression of at least one gene selected from the group comprising ZFP64, DPP3, CCDC43, HIST2H4A/B, KDELC2 and MICALL1 in said sample,
   c) correlating the determined gene expression and/or the change of the gene expression of said at least one gene to an effect of said HDAC inhibitor treatment in said patient.

In certain embodiments of the present invention, the correlation of the determined gene expression and/or the change of the gene expression of said at least one gene to an HDAC inhibitor treatment in a patient may be determined by comparing said determined gene expression and/or change of the gene expression to prior data acquired from other patients, where a certain gene expression and/or change of the gene expression of said at least one gene is already addressed to an effect of said HDAC inhibitor treatment. Such data may for instance be provided in the form of a table or a machine readable data bank.
2. A method of monitoring an HDAC inhibitor treatment, the method comprising the following steps:
   a) Providing a sample of a patient receiving said HDAC inhibitor treatment,
   b) determining the gene expression and/or the change of the gene expression of at least one gene selected from the group comprising ZFP64, DPP3, CCDC43, HIST2H4A/B, KDELC2 and MICALL1 in said sample,
   c) repeating the above steps a and b at least once, preferably more than once, and
   d) using said gene expressions determined in steps a) to c) to generate a time profile of said patient's response to said HDAC inhibitor treatment.

The method of monitoring an HDAC inhibitor treatment according to the present invention may comprise the step of correlating the determined gene expressions and/or the changes of the gene expressions of said at least one gene to an effect of said HDAC inhibitor treatment in said patient.
3. A method according to any of above items 1 or 2, wherein the gene expression and/or the change of the gene expression of said at least one gene is furthermore correlated with the probability of a positive or negative outcome of the HDAC inhibitor treatment.
4. A method of stratification of a patient potentially in need of an HDAC inhibitor treatment comprising the following steps:
   a) Providing a sample of said patient
   b) Determining the gene expression of at least one gene selected from the group comprising ZFP64, DPP3, CCDC43, HIST2H4A/B, KDELC2 and MICALL1 in said sample
   c) Correlating the determined gene expression of said at least one gene to the probability that an HDAC inhibitor treatment has a beneficial effect on said patient and
   d) classifying said patient as responder or non-responder to said HDAC inhibitor treatment, based on the probability determined in step c.
5. The method according to above item 4, wherein said sample provided in step a) is provided before an HDAC inhibitor is administered to said patient,
   wherein after step a) an HDAC inhibitor is added to said sample ex vivo to inhibit HDAC in said sample, and
   wherein in step b) the gene expression of at least one gene is determined in said sample comprising said HDAC inhibitor.

Particularly, in above item 5, the expression “sample provided in step a) is provided before an HDAC inhibitor is administered to said patient” means that said sample provided in step a) is provided before the first time an HDAC inhibitor is administered to said patient. Alternatively, in above item 5, the expression “sample provided in step a) is provided before an HDAC inhibitor is administered to said patient” means that said sample provided in step a) is provided before the first time a specific HDAC inhibitor (e.g. resminostat), which is intended to be administered to said patient for an HDAC inhibitor treatment, is administered to said patient.

In one embodiment, the patient is to be classified as responder if the gene expression of CCDC43 in a sample of said patient differs by 25% or more, preferably 50% or more, more preferably 75% or more, even more preferably 100% or more, compared with the median gene expression of said gene in healthy subjects. In one particular embodiment, said difference is an increase in gene expression, compared with the median gene expression of said gene in healthy subjects. In one particular embodiment, said difference is a decrease in gene expression, compared with the median gene expression of said gene in healthy subjects.

In one embodiment, the patient is to be classified as responder if the gene expression of DPP3 in a sample of said patient differs by 25% or more, preferably 50% or more, more preferably 75% or more, even more preferably 100% or more, compared with the median gene expression of said gene in healthy subjects. In one particular embodiment, said difference is an increase in gene expression, compared with the median gene expression of said gene in healthy subjects. In one particular embodiment, said difference is a decrease in gene expression, compared with the median gene expression of said gene in healthy subjects.

In one embodiment, the patient is to be classified as responder if the gene expression of HIST2H4A/B in a sample of said patient differs by 25% or more, preferably 50% or more, more preferably 75% or more, even more preferably 100% or more, compared with the median gene expression of said gene in healthy subjects. In one particular embodiment, said difference is an increase in gene expression, compared with the median gene expression of said gene in healthy subjects. In one particular embodiment, said difference is a decrease in gene expression, compared with the median gene expression of said gene in healthy subjects.

In one embodiment, the patient is to be classified as responder if the gene expression of KDELC2 in a sample of said patient differs by 25% or more, preferably 50% or more, more preferably 75% or more, even more preferably 100% or more, compared with the median gene expression of said gene in healthy subjects. In one particular embodiment, said difference is an increase in gene expression, compared with the median gene expression of said gene in healthy subjects. In one particular embodiment, said difference is a decrease in gene expression, compared with the median gene expression of said gene in healthy subjects.

In one embodiment, the patient is to be classified as responder if the gene expression of MICALL1 in a sample of said patient differs by 25% or more, preferably 50% or more, more preferably 75% or more, even more preferably 100% or more, compared with the median gene expression of said gene in healthy subjects. In one particular embodiment, said difference is an increase in gene expression, compared with the median gene expression of said gene in healthy subjects. In one particular embodiment, said difference is a decrease in gene expression, compared with the median gene expression of said gene in healthy subjects.

In one embodiment, the patient is to be classified as responder if the gene expression of ZFP64 in a sample of said patient differs by 25% or more, preferably 50% or more, more preferably 75% or more, even more preferably 100% or more, compared with the median gene expression of said gene in healthy subjects. In one particular embodiment, said difference is an increase in gene expression, compared with the median gene expression of said gene in healthy subjects. In one particular embodiment, said difference is a decrease in gene expression, compared with the median gene expression of said gene in healthy subjects.
6. A method of predicting the probability of a positive outcome of an HDAC inhibitor treatment for a patient receiving said HDAC inhibitor treatment, the method comprising the following steps:
   a) Providing a sample of said patient
   b) Determining the gene expression of at least one gene selected from the group comprising ZFP64, DPP3, CCDC43, HIST2H4A/B, KDELC2 and MICALL1 in said sample,
   c) Comparing said gene expression with the gene expression of said at least one gene in a sample provided from said patient prior to step a), and
   d) Correlating the difference of the gene expression of said at least one gene in said sample provided in step a) and in said sample provided prior to step a) to the probability of a positive outcome of said HDAC inhibitor treatment for said patient.
7. A method according to above item 6, wherein said sample provided prior to step a) is provided from said patient before an HDAC inhibitor is administered to said patient, and wherein said sample provided in step a) is provided after an HDAC inhibitor is administered to said patient, preferably after an HDAC inhibitor is administered to said patient for the first time, wherein, preferably, “before an HDAC inhibitor is administered” means 1 second to one day, more preferably one second to one hour before said HDAC inhibitor is administered.

In one embodiment, the probability of a positive outcome of an HDAC inhibitor treatment for a given patient is 75% or greater, preferably 85% or greater, more preferably 90% or greater, even more preferably 95% or greater, if the gene expression of CCDC43, determined two hours after administration of an HDAC inhibitor, is changed by 25% or more, preferably 50% or more, more preferably 75% or more, even more preferably 100% or more, yet even more preferably 150% or more, compared with the gene expression of said gene determined in a sample from said patient before an HDAC inhibitor is administered to said patient. In one particular embodiment, said change is an increase in gene expression. In one particular embodiment, said change is a decrease in gene expression.

In one embodiment, the probability of a positive outcome of an HDAC inhibitor treatment for a given patient is 75% or greater, preferably 85% or greater, more preferably 90% or greater, even more preferably 95% or greater, if the gene expression of DPP3, determined two hours after administration of an HDAC inhibitor, is changed by 25% or more, preferably 50% or more, more preferably 75% or more, even more preferably 100% or more, yet even more preferably 150% or more, compared with the gene expression of said gene determined in a sample from said patient before an HDAC inhibitor is administered to said patient. In one particular embodiment, said change is an increase in gene expression. In one particular embodiment, said change is a decrease in gene expression.

In one embodiment, the probability of a positive outcome of an HDAC inhibitor treatment for a given patient is 75% or greater, preferably 85% or greater, more preferably 90% or greater, even more preferably 95% or greater, if the gene expression of HIST2H4A/B, determined two hours after administration of an HDAC inhibitor, is changed by 25% or more, preferably 50% or more, more preferably 75% or more, even more preferably 100% or more, yet even more preferably 150% or more, compared with the gene expression of said gene determined in a sample from said patient before an HDAC inhibitor is administered to said patient. In one particular embodiment, said change is an increase in gene expression. In one particular embodiment, said change is a decrease in gene expression.

In one embodiment, the probability of a positive outcome of an HDAC inhibitor treatment for a given patient is 75% or greater, preferably 85% or greater, more preferably 90% or greater, even more preferably 95% or greater, if the gene expression of KDELC2, determined two hours after administration of an HDAC inhibitor, is changed by 25% or more, preferably 50% or more, more preferably 75% or more, even more preferably 100% or more, yet even more preferably 150% or more, compared with the gene expression of said gene determined in a sample from said patient before an HDAC inhibitor is administered to said patient. In one particular embodiment, said change is an increase in gene expression. In one particular embodiment, said change is a decrease in gene expression.

In one embodiment, the probability of a positive outcome of an HDAC inhibitor treatment for a given patient is 75% or greater, preferably 85% or greater, more preferably 90% or greater, even more preferably 95% or greater, if the gene expression of MICALL1, determined two hours after administration of an HDAC inhibitor, is changed by 25% or more, preferably 50% or more, more preferably 75% or more, even more preferably 100% or more, yet even more preferably 150% or more, compared with the gene expression of said gene determined in a sample from said patient before an HDAC inhibitor is administered to said patient. In one particular embodiment, said change is an increase in gene expression. In one particular embodiment, said change is a decrease in gene expression.

In one embodiment, the probability of a positive outcome of an HDAC inhibitor treatment for a given patient is 75% or greater, preferably 85% or greater, more preferably 90% or greater, even more preferably 95% or greater, if the gene expression of ZFP64, determined two hours after administration of an HDAC inhibitor, is changed by 25% or more, preferably 50% or more, more preferably 75% or more, even more preferably 100% or more, yet even more preferably 150% or more, compared with the gene expression of said gene determined in a sample from said patient before an HDAC inhibitor is administered to said patient. In one particular embodiment, said change is an increase in gene expression. In one particular embodiment, said change is a decrease in gene expression.
8. A method of determining the gene expression of at least one gene as pharmacodynamic marker in a patient in need of an HDAC inhibitor treatment, the method comprising the following steps:
   a) Providing a sample of said patient,
   b) determining the gene expression and/or the ZFP64, DPP3, CCDC43, HIST2H4A/B, KDELC2 and MICALL1 in said sample
   c) correlating the determined gene expression and/or the change of the gene expression of said at least one gene to the relative inhibition of HDAC by the HDAC inhibitor.
9. The method according to any of above items 1 to 8, wherein the gene expression of said at least one gene is determined by measuring the level of at least one mRNA encoded by said at least one gene or a fragment thereof of at least 150 nucleotides in length, preferably at least 180 nucleotides in length, in said sample.
10. The method according to any of above items 1 to 8, wherein the gene expression of said at least one gene is determined by measuring the level of at least one protein encoded by said at least one gene, or a domain of said protein, in said sample.
11. The method according to above item 10, wherein the level and/or the change of the level of said at least one protein or domain thereof is determined by the binding of an antibody or a probe comprising an antibody, wherein said antibody specifically binds to said at least one protein or domain thereof.
12. The method according to any of above items 4 to 11, wherein the sample is taken either before starting of the HDAC inhibitor treatment or during HDAC inhibitor treatment.
13. A method according to any of above items 1 to 12, wherein said sample is a sample of a bodily fluid, preferably a blood sample selected from the group comprising whole blood, serum or plasma, more preferably a peripheral blood sample selected from the group comprising whole blood, serum or plasma.
14. A method according to any of above items 1 to 12, wherein the sample is a tissue sample, preferably a sample of diseased tissue, more preferably a biopsy from cancer tissue.
15. The method according to any of above items 1 to 14, wherein steps a to c or a to b are repeated at least once, preferably more than once.

In the methods of the present invention, where steps a to c or a to b are repeated, typically, said steps are repeated after each administration of an HCAD inhibitor or after each treatment cycle. Alternatively steps a to c or a to b may be repeated in less frequent intervals, such as after each second, third, fourth, etc. administration of an HCAD inhibitor or after each second, third, fourth, etc. treatment cycle. In this way, the course of the treatment and the patient's health state can be monitored.
16. A method according to any of the preceding above items 1 to 15, wherein the HDAC inhibitor is selected from the group comprising vorinostat, romidepsin, valproic acid, panobinostat, entinostat, belinostat, mocetinostat, givinostat and resminostat or a pharmaceutically acceptable salt thereof, preferably (E)-3-(1-(4-((dimethylamino)methyl)phenylsulfonyl)-1H-pyrrol-3-yl)-N-hydroxyacrylamide in free form or the hydrochloride or mesylate salt thereof.
17. The use of at least one gene or the use of a protein encoded by said at least one gene, wherein said at least one gene is selected from the group comprising ZFP64, DPP3, CCDC43, HIST2H4A/B, KDELC2 and MICALL1 as a pharmacodynamic marker in an HDAC inhibitor treatment for a patient in need of said HDAC inhibitor treatment.
18. The use of at least one gene or the use of a protein encoded by said at least one gene, wherein said at least one gene is selected from the group comprising ZFP64, DPP3, CCDC43, HIST2H4A/B, KDELC2 and MICALL1 for predicting the outcome of an HDAC inhibitor treatment for a patient in need of said HDAC inhibitor treatment.
19. The use of at least one gene or the use of a protein encoded by said at least one gene, wherein said at least one gene is selected from the group comprising ZFP64, DPP3, CCDC43, HIST2H4A/B, KDELC2 and MICALL1 as a surrogate marker for determining HDAC activity.
20. The use of at least one gene or the use of a protein encoded by said at least one gene, wherein said at least one gene is selected from the group comprising ZFP64, DPP3, CCDC43, HIST2H4A/B, KDELC2 and MICALL1 for stratifying a patient potentially in need of an HDAC inhibitor treatment as responder or non-responder.
21. A kit for determining the gene expression of at least one gene selected from the group comprising ZFP64, DPP3, CCDC43, HIST2H4A/B, KDELC2 and MICALL1 in a sample,
   wherein the kit comprises probes which specifically bind to at least one mRNA encoded by said at least one gene or a fragment thereof of at least 150 nucleotides in length, preferably at least 180 nucleotides in length, and
   wherein the kit optionally comprises one or more further components selected from the group comprising media, medium components, buffers, buffer components, RNA purification columns, DNA purification columns, dyes, nucleic acids including dNTP mix, enzymes including polymerases, and salts.
22. A kit for determining the level of at least one protein encoded by a gene selected from the group comprising ZFP64, DPP3, CCDC43, HIST2H4A/B, KDELC2 and MICALL1 in a sample:
   wherein the kit comprises probes which specifically bind to at least one protein encoded by said at least one gene or a domain of said protein, and
   wherein the kit optionally comprises one or more further components selected from the group comprising media, medium components, buffers, buffer components, membranes, ELISA plates enzyme substrates, dyes, enzymes including polymerases, and salts.
23. The use of a kit according to above item 21 or 22 for determining the gene expression of at least one gene selected from the group comprising ZFP64, DPP3, CCDC43, HIST2H4A/B, KDELC2 and MICALL1 in a sample.
24. The use according to above item 23, wherein said determined gene expression is correlated to HDAC activity in said sample.
25. The use according to above item 23 or 24, wherein said sample is provided from a patient potentially in need of an HDAC inhibitor treatment.
26. The use of a kit according to above item 21 or 22 in a method according to any of above items 1 to 16.
27. An HDAC inhibitor for use in the treatment of a patient potentially in need of an HDAC inhibitor treatment, wherein before and/or during said treatment at least one gene selected from the group comprising ZFP64, DPP3, CCDC43, HIST2H4A/B, KDELC2 and MICALL1, at least one mRNA corresponding to said at least one gene, or at least one protein encoded by said at least one gene is used for determining the probability of an effect of the HDAC inhibitor treatment to said patient, or for determining whether said patient is a responder to the HDAC inhibitor treatment.
28. A method of treating a patient potentially in need of an HDAC inhibitor treatment, the method comprising administering to the patient an HDAC inhibitor, wherein before and/or during said method at least one gene selected from the group comprising ZFP64, DPP3, CCDC43, HIST2H4A/B, KDELC2 and MICALL1, at least one mRNA corresponding to said at least one gene, or at least one protein encoded by said at least one gene is used for determining the probability of an effect of the HDAC inhibitor treatment to said patient, or for determining whether said patient is a responder to the HDAC inhibitor treatment.
29. The HDAC inhibitor for use in the treatment of a patient potentially in need of an HDAC inhibitor treatment according to above item 27 or the method according to above item 28 wherein the HDAC inhibitor is selected from the group comprising vorinostat, romidepsin, valproic acid, panobinostat, entinostat, belinostat, mocetinostat, givinostat and resminostat or a pharmaceutically acceptable salt thereof, preferably (E)-3-(1-(4-((dimethylamino)methyl)phenylsulfonyl)-1H-pyrrol-3-yl)-N-hydroxyacrylamide in free form or a hydrochloride salt or a mesylate salt thereof.

Particularly preferred embodiments of the present invention relate to the respective methods, uses, kits and HDAC inhibitors for use in the treatment of a patient potentially in need of an HDAC inhibitor treatment as described above, wherein the gene selected from the group comprising ZFP64, DPP3, CCDC43, HIST2H4A/B, KDELC2 and MICALL1 is ZFP64.

More preferred are methods for predicting the probability of a positive outcome of an HDAC inhibitor treatment for a patient receiving an HDAC inhibitor treatment, determining the probability of a certain effect of the HDAC inhibitor treatment, monitoring an HDAC inhibitor treatment and/or the stratification of a patient potentially in need of a HDAC inhibitor treatment as described herein, wherein the gene selected from the group comprising ZFP64, DPP3, CCDC43, HIST2H4A/B, KDELC2 and MICALL1 is selected from the group comprising ZFP64 and DPP3.

Even more preferred is a method of predicting the probability of a positive outcome of an HDAC inhibitor treatment for a patient receiving said HDAC inhibitor treatment as, the method comprising the following steps:
a) Providing a sample of said patient
b) Determining the gene expression of ZFP64 in said sample,
c) Comparing said gene expression with the gene expression of ZFP64 in a sample provided from said patient prior to step a), and
   correlating the difference of the gene expression of ZFP64 in said sample provided in step a) and in said sample provided prior to step a) to the probability of a positive outcome of said HDAC inhibitor treatment for said patient. Even more particularly preferred embodiments of the present invention relate to the preferred methods of predicting the probability of a positive outcome of an HDAC inhibitor treatment for a patient receiving said HDAC inhibitor treatment as described above, wherein said gene is ZFP64.

Furthermore even more preferred is a method of predicting the probability of a positive outcome of an HDAC inhibitor treatment for a patient receiving said HDAC inhibitor treatment as, the method comprising the following steps:
d) Providing a sample of said patient
e) Determining the gene expression of DPP3 in said sample,
f) Comparing said gene expression with the gene expression of DPP3 in a sample provided from said patient prior to step a), and
   correlating the difference of the gene expression of DPP3 in said sample provided in step a) and in said sample provided prior to step a) to the probability of a positive outcome of said HDAC inhibitor treatment for said patient. Even more particularly preferred embodiments of the present invention relate to the preferred methods of predicting the probability of a positive outcome of an HDAC inhibitor treatment for a patient receiving said HDAC inhibitor treatment as described above, wherein said gene is DPP3.

In the embodiments of the present invention, e.g. the uses, methods, kits and HDAC inhibitors according to the present invention, where applicable, the patient is preferably a patient suffering from cancer, particularly from hematological cancer, more particularly from Hodgkin's Lymphoma, and where applicable, the sample is preferably obtained from a patient suffering from cancer, particularly from hematological cancer, more particularly from Hodgkin's Lymphoma.

In other embodiments of the present invention, e.g. the uses, methods, kits and HDAC inhibitors according to the present invention, where applicable, the patient is preferably a patient suffering from CRC or HCC, more preferably HCC, and where applicable, the sample is preferably obtained from a patient suffering from CRC or HCC, more preferably HCC.

In one particular embodiment of the present invention the gene expression of one or more of the biomarkers according to the present invention is measured at multiple time points after administration of an HDAC inhibitor to the patient. In this manner, a time profile of the change of gene expression of the biomarkers can be determined, which may increase the biomarker's validity. Generally, the one or more of the biomarkers according to the present invention is/are measured at multiple time points after administration of an HDAC inhibitor to the patient. Preferably, the one or more of the biomarkers according to the present invention is/are measured at at least three, or at least four, or at least five, or at least six time points after administration of an HDAC inhibitor to the patient.

In the methods according to the present invention the gene expression of said one or more genes is preferably an indicator for the inhibition of HDAC by the HDAC inhibitor.

In the methods according to the present invention the gene expression of said one or more genes is preferably correlated with the outcome of the HDAC inhibitor treatment.

In certain embodiments of the methods according to the present invention the sample is taken either before starting the HDAC inhibitor treatment or during HDAC inhibitor treatment, as appropriate in the respective method.

Preferably the kits according to the present invention are used for determining the level of the at least one gene according to the present invention or at least one protein encoded by said at least one gene according to the present invention in a sample of a patient in need of a HDAC inhibitor treatment.

As used herein, the term “HDAC”, or histone deacetylase, specifies an enzyme which facilitates deacetylation of the histone, and which may furthermore facilitate deacetylation of other proteins, such as transcription factors, receptors, etc. The family of HDAC proteins includes proteins transcribed from the following human genes, which are defined by their NCI Gene IDs, as well as their counterparts in other mammalian species: HDAC1, ID: 3065; HDAC2, ID: 3066; HDAC3, ID: 8841; HDAC4, ID: 9759; HDAC5, ID: 10014; HDAC6, ID: 10013; HDAC7, ID: 51564; HDAC8, ID: 55869; HDAC9, ID: 51564; HDAC10, ID: 83933; HDAC11, ID: 79885; SIRT1, ID: 23411; SIRT2, ID: 22933; SIRT3, ID: 23410; SIRT4, ID: 23409; SIRT5, ID: 23408; SIRT6, ID: 51548; SIRT7, ID: 51547.

The gene sequences specified herein by Entrez ID and Official gene symbol (NCBI) relate to the human genes. However, the present invention also encompasses the corresponding genes in other mammalian species for applications wherein the patient is a non-human mammal.

As used herein, the term “HDAC inhibitor of the hydroxamate type” specifies an HDAC inhibitor comprising a hydroxamate group which is capable of chelating the zinc ion situated in the active site of HDAC.

In all embodiments of the present invention, including all methods, uses, compounds for use, kits, etc., the HDAC inhibitor is particularly resminostat (e.g. “HDAC inhibitor treatment” then is particularly “resminostat treatment”.

In all embodiments of the present invention, including all methods, uses, compounds for use, kits, etc., the gene selected from the group comprising ZFP64, DPP3, CCDC43, HIST2H4A/B, KDELC2 and MICALL1 is particularly ZFP64.

As used herein, the term “inhibition” specifies that the activity of the entity which is to be inhibited is diminished, e.g. in the case of an enzyme, e.g. HDAC, that the turnover rate of the substrate conversion by the enzyme is diminished.

As used herein, the term “relative inhibition” in the context of HDAC inhibition by the HDAC inhibitor means the inhibition of HDAC activity relative to the predose HDAC activity level, i.e. before administration of the HDAC inhibitor.

As used herein, the term “level of inhibition of HDAC” or “level of HDAC inhibition” relates to the ratio by which HDAC activity is reduced upon administration of an inhibitor. High level of inhibition of HDAC would mean that HDAC activity is strongly reduced, whereas low level of inhibition of HDAC would mean that HDAC activity is only slightly reduced, in each case compared with the HDAC activity before administration of an inhibitor.

As used herein, the term “gene expression” refers to the amount of an expression product of a gene in a cell. Expression products include transcripts of the gene, e.g. mRNA, and the corresponding translation products, i.e. proteins. The gene expression is often indicated as relative expression level, i.e. the expression of a target gene at a given timepoint relative to the expression of one or more housekeeping genes and/or relative to the expression of said target gene at a specified different timepoint, which usually is the timepoint before administering the drug, e.g. before administering the drug for the first time, or before administering the drug for the first time in a given treatment cycle. The gene expression specifies the relative abundance of a target gene within a certain sample. “Gene expression” therefore may also include the absence of expression of a given gene. Absolute values for gene expressions are usually expressed as “Ct” value (see herein below) and may vary for each gene, depending on the particular method with which expression is determined, in particular on the polymerases used.

As used herein, the expression “selected from the group comprising (e.g. item x, item y and item z)”, and the like, are preferably equivalent to “selected from (e.g. item x, item y and item z)” (wherein the term “and” is not meant to be understood that all of the aforementioned items—e.g. item x, item y and item z—are to be selected, but rather that one (or more, depending on the specific context) of the items of said group is to be selected). In particular embodiments, this also includes “selected from the group consisting of (e.g. item x, item y and item z)”.

In table 2 data are presented which define discrete expression ranges for the expression of the genes according to the present invention, as described above. Herein, a correlation with an outcome of an HDAC inhibitor treatment can be made in ranges 2 and 3 with a certain confidence, and preferably, a correlation with an outcome of an HDAC inhibitor treatment can be made in ranges 1 and 4 with higher confidence.

TABLE 2
Definition of levels of gene expressions given in dCt values

| | range 1 | range 2 | range 3 | range 4 |
|---|---|---|---|---|
| CCDC43 | <9.44 | 9.45-10.00 | 10.01-10.70 | >10.71 |
| DPP3 | >9.30 | 9.31-9.92 | 9.93-10.20 | >10.21 |
| HIST2H4A and | <1.61 | 1.6-2.29 | 2.30-3.03 | >3.04 |
| HIST2H4B | | | | |
| KDELC2 | <11.80 | 11.81-12.53 | 12.54-13.23 | >13.23 |
| MICALL1 | <9.33 | 9.34-9.99 | 10.00-11.03 | >11.04 |
| ZFP64 | <10.94 | 10.94-11.87 | 11.88-12.63 | >12.64 |

In Table 2a data are presented which particularly define discrete expression ranges for the expression of the ZFP64 genes according to the present invention, as described above. Herein, a correlation with an outcome of an HDAC inhibitor treatment can be made in range 2 with a certain confidence, and preferably, a correlation with an outcome of an HDAC inhibitor treatment can be made in ranges 1 and 3 with higher confidence

TABLE 2a
Particular levels of ZFP64 gene expressions given in dCt values

| | range 1 | range 2 | range 3 |
|---|---|---|---|
| ZFP64 | <10.02 | 10.02-11.15 | >11.15 |

As used herein, the term “baseline gene expression” specifies a level of expression of a gene, RNA or protein which corresponds to the mean or average level determined in a population of individuals, wherein said individuals are not under the influence of an HDAC inhibitor. Said population may reflect the demographic composition of the overall population, or a specific sub-population, wherein the sub-population comprises individuals which are selected on one or more factors selected from the group comprising medical condition, including whether the individual is suffering from a specific disease, such as cancer or a certain cancer type, has a certain degree of severity of the disease, or is healthy; gender; ethnicity; body mass index; prior history of medical conditions; age; certain factors in the individual's lifestyle, such as alcohol or substance use, smoking, medication, nutrition, etc.

As used herein, the term “change of the gene expression” relates to a change in the gene expression of a gene at a given time point, relative to the gene expression of said gene at a different time point, preferably relative to the gene expression of said gene at an earlier time point. This may for instance refer to a change in the gene expression with respect to a baseline gene expression (compare above), a predose gene expression (i.e. the gene expression for the same individual before administration of an HDAC inhibitor) or the gene expression measured at a time point before, after or during treatment. In certain cases, the change of the gene expression of a gene may be zero (i.e. the gene expression does not change) or not detectable; such cases are however also encompassed in embodiments relating to a change of the gene expression, where the result of determining the change of the gene expression is that the gene expression remains unchanged.

For instance, the change of the gene expression may be determined (i) by comparing the gene expression of a given gene prior to start of treatment with an HDAC inhibitor to the gene expression of said gene during HDAC inhibitor treatment; (ii) by comparing the gene expression of a given gene at one time point during HDAC inhibitor treatment to the gene expression of said gene at another time point during HDAC inhibitor treatment; and/or (iii) by comparing the gene expression of a given gene with a baseline gene expression of said gene determined from a population of healthy, diseased, untreated and/or treated individuals.

Said change of the gene expression may relate to an increase or a decrease of gene expression, i.e. an up- or downregulation of the gene. Moreover, the genes according to the present invention may be up- or downregulated independently from one another, e.g. one or more of said genes may be upregulated, whereas others may be downregulated and whereas the gene expression of other genes may remain unchanged.

As used herein, the term “biomarker” specifies a molecular species, such as a polypeptide, e.g. a protein, or a polynucleic acid, e.g. mRNA that can be detected and evaluated as an indicator of normal biologic processes, pathogenic processes, or pharmacologic responses to a therapeutic intervention. As such, a biomarker is a measurable characteristic that reflects physiological, pharmacological, or disease processes or disease states.

Unless specified otherwise, terms such as “biomarker” or “a biomarker” includes a combination of more than one biomarker. This means that in certain cases the combined data gathered for more than one biomarker may be indicative for a certain effect of HDAC inhibitor treatment. Moreover, the predictive or prognostic value of the data gathered for one or more biomarkers according to the present invention may be enhanced by taking further biomarkers, such as baseline HDAC activity or baseline level of histone or protein acetylation, or other biomarkers commonly used in medicine, such body mass index, prior history of disease, age, etc.

As used herein, a diagnostic biomarker is a biomarker as described above which is used to identify the presence, severity or absence of a specific disease state.

As used herein, a prognostic biomarker is a biomarker as described above which is used to determine a patient's survival probability.

As used herein, the term “pharmacodynamic biomarker” or “pharmacodynamic marker” specifies a biomarker as described above which, by the change of its level upon administration of a drug, e.g. an HDAC inhibitor, indicates the presence and/or an effect of the drug in the patient. The drug may be present in the patient's overall system or in a specific pharmacological compartment of the patient, such as e.g. in the patient's blood, body fluids, liver, fatty tissue or other tissues. The pharmacodynamic biomarker may be used in the testing of novel drugs to determine whether said drug hits the target in vivo, i.e. whether there is any HDAC inhibition in vivo.

Furthermore, the pharmacodynamic biomarker may be used for pharmacokinetic/pharmacodynamic modeling (PK/PD modeling), i.e. for correlating pharmacokinetic behavior with pharmacodynamic behavior, which relates to a correlation of the administrated dose of an HDAC inhibitor to the level of inhibition in vivo, e.g. in preclinical Phase I clinical trials. Furthermore, the pharmacodynamic biomarker may also be used for dose finding in vivo, e.g. in a preclinical model study for a Phase I clinical trial or by using the data collected in a Phase I clinical trial for dose finding in a Phase II clinical trial.

As used herein, a predictive biomarker is a biomarker as described above which is used to identify which patient is likely or unlikely to benefit from a particular treatment, e.g. discriminating a responder from a non-responder. A predictive biomarker can be used before administration of the drug for patient stratification or during treatment monitoring, wherein the monitoring data is used to predict the further outcome of the treatment. Predictive biomarkers may also serve as a surrogate endpoint, i.e. to determine whether treatment should be continued.

As used herein, “stratification” or “stratifying” relates to the use of a biomarker according to the present invention for selection of patients depending on their predose biomarker level, i.e. the level of one or more of the biomarkers according to the present invention before administration of an HDAC inhibitor, thereby determining the probability that a certain patient will benefit from an HDAC inhibitor treatment.

As used herein, the term “housekeeping gene” specifies typically one or more constitutive genes that show a good detectable expression by the used technique, e.g. in the present invention Ct values <25 in the qPCR technique with an amount of at least 100 ng total RNA. Similar considerations apply of course in the case wherein the expression is determined on the protein level. Furthermore, the housekeeping gene shows none to minimal changes in gene expressions upon administration of the drug, in all samples of a specific patient group receiving equal treatment regimens. The expression of the housekeeping gene or the housekeeping genes is used to normalize deviation in the determined results, caused e.g. by individual differences in the respective samples, such as different cell numbers, and/or by technical aspects e.g. pipetting errors.

As used herein, the term “target gene” specifies the gene of interest that is investigated in this test system for its expression and regulation by a certain treatment.

As used herein, the term “HDAC inhibitor treatment” or means that a patient in need thereof receives a treatment regimen encompassing the administration of one or more doses of an HDAC inhibitor in order to treat a medical condition in said patient (e.g. disease), as detailed further in the description of the present invention. The HDAC inhibitor treatment as defined herein may encompass the period of time beginning from the diagnosis of a medical condition, and including the regimen of treatment, until the last follow-up examination, i.e. wherein the patient does not receive any more medication but the patient's physical condition and state of the treatment are controlled. In certain cases, the follow-up may encompass the determination of long-term effects of the administration of an HDAC inhibitor, which may be present even months or years after the last administration of an HDAC inhibitor to a given patient.

In this context “treatment cycle” refers to a period of time during which an HDAC inhibitor is administered to the patient in certain specific time intervals, and which may comprise a certain time period wherein no HDAC inhibitor is administered to the patient so that the HDAC inhibitor is completely excreted from said patient. For example, a treatment cycle may comprise 14 days, wherein an HDAC inhibitor is administered twice daily on days 1 to 5 and wherein no HDAC inhibitor is administered on days 6 to 14. The treatment cycle is usually repeated at least once, preferably more than once, during the HDAC inhibitor treatment, which may however depend on a number of factors, such as for example the patient's response to the administration of the HDAC inhibitor, the occurrence of unwanted side effects of the HDAC inhibitor, the patient's overall health state, etc.

As used herein, the term “effect” in the context of an “effect of an HDAC inhibitor treatment” or “effect of the HDAC inhibitor treatment” includes a pharmacodynamic effect and/or a positive or negative outcome of HDAC inhibitor treatment as defined herein below. Examples for such effects are a) a pharmacodynamic effect, i.e. an effect on the molecular level, including effects selected from the group comprising reduction of HDAC activity, induction of histone acetylation or acetylation of other proteins, such as transcription factors or receptors, modulation of gene transcription, modulation of protein expression and modulated activity of signaling pathways; b) an effect on the diseased tissue or cells including changes in tumor size, metabolic activity, cell viability, blood supply of the tumor, i.e. angiogenesis, composition of the tumor, e.g. relationship of cells comprising the tumor e.g. tumor cells, immune cells, fibroblasts and endothelial cells; and c) an effect on the patient's medical state including changes in clinical status, health status, progression or stabilization of disease, decreased or increased time of progression free survival, cure of disease, enhanced or shortened overall survival, delay of disease progression and alleviation or aggravation of symptoms. In a preferred embodiment of the present invention, the effect is a pharmacodynamics effect.

As used herein, the term “positive outcome of HDAC inhibitor treatment” means that the HDAC inhibitor treatment results in a beneficial effect for the patient. This includes clinical benefit, health improvement, stabilization of disease, increased time of progression free survival, cure of disease, enhanced overall survival, and delay of disease progression and alleviation of symptoms.

As used herein, the term “negative outcome of HDAC inhibitor treatment” means that the HDAC inhibitor treatment does not result in a beneficial effect for the patient or that the outcome is the opposite of the aforementioned positive outcome, e.g. health decline.

As used herein, a “responder” is a patient who shows a positive outcome due to HDAC inhibitor treatment as defined above.

As used herein, a “non-responder” is a patient who shows a negative outcome due to HDAC inhibitor treatment as defined above.

As used herein, the term “bodily fluid or body fluid” specifies a fluid or part of a fluid originating from the body of a patient, including fluids that are excreted or secreted from the body of the patient, including but not limited to blood, including peripheral blood, serum, plasma, urine, interstitial fluid, liquor, aqueous humour and vitreous humour, bile, breast milk, cerebrospinal fluid, endolymph, perilymph, ejaculate, gastric juice, mucus, peritoneal fluid, pleural fluid, saliva, sweat, tears and vaginal secretion. Preferred bodily fluids in the context of the present invention are peripheral blood, serum, plasma and urine. Said bodily fluid itself may or may not comprise diseased and/or non-diseased cells.

As used herein, the term “tissue sample” specifies a non-fluid material or solid originating from the body of a patient. Tissue samples include, but are not limited to samples of bone material, bone marrow, skin, hair follicle, mucosa, brain, cartilage, muscles, lung, kidney, stomach, intestines, bladder and liver. Said tissue sample itself may or may not comprise diseased cells, and may for instance be a sample taken from a diseased region of a patient's body, such as a biopsy of a tumor. Preferably the tissue sample is selected from skin, hair follicle or oral mucosa.

In the embodiments of the present invention, the sample is obtained from the patient by any method and/or means commonly known to the skilled person in the field of medicine, e.g. preferably blood sample taking by venipuncture.

As used herein, the term “peripheral blood” specifies blood obtained from the circulation remote from the heart, i.e. the blood in the systemic circulation, as for example blood from acral areas.

As used herein, the term “whole blood” specifies unmodified blood comprising cells and fluid, as obtained from the donor of said blood, such as a patient.

As used herein, the term “patient” specifies a subject which is intended to receive HDAC inhibitor treatment. Patients are potentially diseased and may include diseased and healthy subjects, e.g. healthy volunteers in Phase I clinical trials to determine safety, toxicity and pharmacodynamic behavior of an HDAC inhibitor. Preferably, the patient is a mammal, more preferably a human. In a further preferred embodiment the patient is suffering from cancer.

As used herein, the term “patient potentially in need of an HDAC inhibitor treatment” specifies a subject suspected of having a disease or disorder, preferably having a disease or disorder, for which an HDAC inhibitor treatment is expected to be beneficial and/or which is responsive to an HDAC inhibitor treatment. In this context, “patient potentially in need” also includes and in particular embodiments means “patient in need”.

The expression of the genes according to the present invention can be measured using detection methodology according to the state of the art for quantification of mRNA derived from transcription processes of these genes, such as quantitative Real-time PCR (qPCR) approaches. Furthermore, these gene expressions can be measured by analyzing the expression of proteins encoded by the genes in question. All of those measurements may be performed ex vivo or in vitro.

The methods for detection or measurement of RNA are not particularly limited and detection or measurement of RNA may be conducted by any suitable method known to the skilled person. Examples of such methods are quantitative PCR (also known as real time PCR), quantitative sequencing of mRNA (also known as deep sequencing), Northern blot technique or dot blot technique. The above methods may entail the use of certain specific probes comprising primer pairs and/or comprising a DNA molecule. Such primer pairs are typically a pair of short, non-complementary single stranded DNA molecules, e.g. of about 20 bases in length, which bind specifically to different regions of the same polynucleic acid molecule of interest (typically a cDNA copy of a certain mRNA molecule) and may be used in the amplification of said polynucleic acid. The aforementioned molecular probe comprising a DNA molecule is a molecular construct comprising a single stranded DNA molecule, which specifically binds to the polynucleic acid molecule of interest, and one or more labels (also known as “tags”) to facilitate detection. Optionally, said probes may further comprise one or more linker moieties. The aforementioned labels may for instance be selected from color labels which show a change in color intensity upon binding of the single stranded DNA molecule, fluorescence labels, such as fluorescent proteins or fluorescent dyes, enzymatic labels, such as horseradish peroxidase, radioactive labels or other labels allowing for detection of the binding of the single stranded DNA molecule which are commonly applied in molecular biology.

In the context of the present invention a “probe comprising an antibody” is a molecular construct comprising a specific antibody for binding to the epitope and one or more labels (also known as “tags”) to facilitate detection. Optionally, said probes may further comprise one or more linker moieties. The aforementioned labels may for instance be selected from color labels which indicate binding of the antibody based on color intensity and/or change, fluorescence labels, such as fluorescent proteins or fluorescent dyes, enzymatic labels, such as horseradish peroxidase, radioactive labels or other labels allowing for detection of antibody binding which are commonly applied in molecular biology. Other possibilities of detecting the binding of a specific antibody to its target epitope include indirect techniques wherein for detection of the specific antibody a second antibody is used, wherein the second antibody carries a label, the label being as defined above, and wherein the second antibody specifically binds to the aforementioned specific antibody. Such indirect techniques include methods commonly known in the field of molecular biology, such as ELISA, HPLC methods for the detection of proteins, Western Blot technique, reversed phase protein detection technique or dot blot technique. The aforementioned “indirect techniques” may also be performed in a direct setting, wherein the aforementioned probes bind to the target epitope and are detected directly. In certain embodiments, the specific antibody may be immobilized, e.g. on a sheet material, on beads, strips, etc. In one embodiment, the detection is facilitated in solution or with beads suspended in a solution, said beads comprising immobilized probes as mentioned herein.

As used herein, the term “probes” also refers to “a probe”, i.e. a single probe.

In a further embodiment detection and/or quantification of proteins may be facilitated by mass spectrometry methods, or LC-coupled mass spectrometry methods.

Exemplary methods for use in the present invention are described in detail in “Short Protocols in Molecular Biology”, 5th Edition, 2 Volume Set; Frederick M. Ausubel (Editor), Roger Brent (Editor), Robert E. Kingston (Editor), David D. Moore (Editor), J. G. Seidman (Editor), John A. Smith (Editor), Kevin Struhl (Editor); Wiley; ISBN: 978-0-471-25092-0.

As defined herein, a specifically binding antibody, primer pair or DNA molecule preferably has a binding affinity to its target structure of at least 1000-fold relative to other structures. “Structure” herein relates to molecular entities including protein epitopes and polynucleic acid sequences. Herein, “epitope” is the part of a protein that is recognized by an antibody.

Antibodies for use in the present invention may be obtained by any method known to a person skilled in the art. The type of said antibodies is not particularly limited and in principle, any antibody type suitable for the detection of the expression product of a gene can be applied, including monoclonal antibodies and polyclonal antibodies.

The methods, uses and kits according to the present invention are applicable in HDAC inhibitor treatment, including preparation and follow-up thereof, of diseases or disorders which are responsive to the inhibition of HDAC. Such diseases and disorders include cellular neoplasia, which is defined by cells displaying aberrant cell proliferation and/or survival and/or a block in differentiation. The term neoplasia includes “benign neoplasia” which relates to hyperproliferation of cells, incapable of forming an aggressive, metastasizing tumor in vivo and “malignant neoplasia”, which relates to cells with multiple cellular and biochemical abnormalities, capable of forming a systemic disease, for example forming tumor metastases in distant organs. Examples of malignant neoplasia include solid and hematological tumors. Solid tumors are exemplified by tumors of the breast, bladder, bone, brain, central and peripheral nervous system, colon, endocrine glands (e.g. thyroid and adrenal cortex), esophagus, endometrium, germ cells, head and neck, kidney, liver, lung, larynx and hypopharynx, mesothelioma, ovary, pancreas, prostate, rectum, renal, small intestine, soft tissue, testis, stomach, skin, ureter, vagina and vulva. Malignant neoplasias include inherited cancers exemplified by Retinoblastoma and Wilms tumor. In addition, malignant neoplasias include primary tumors in said organs and corresponding secondary tumors in distant organs (“tumor metastases”). Hematological tumors are exemplified by aggressive and indolent forms of leukemia and lymphoma, namely non-Hodgkins disease, chronic and acute myeloid leukemia (CML/AML), chronic and acute lymphoblastic leukemia (CLL/ALL), Hodgkin's disease, multiple myeloma and T-cell lymphoma. Also included are myelodysplastic syndrome, plasma cell neoplasia, paraneoplastic syndromes, cancers of unknown primary site as well as AIDS related malignancies.

Particularly preferred embodiments of the present invention relate to cancers selected from the group consisting of liver including HCC and liver cancer metastases, pancreas, GI tract including colon and colorectal (CRC), thyroid, kidney (i.e. renal cancer), skin, testis and lymphoma including Hodgkin lymphoma. In such particularly preferred embodiments, the patient according to the present invention is preferably a cancer patient, more preferably a patient suffering from a cancer selected from the group consisting of liver including HCC and liver cancer metastases, pancreas, GI tract including colon and colorectal (CRC), thyroid, kidney (i.e. renal cancer), and lymphoma including Hodgkin lymphoma.

Furthermore, diseases or disorders which are responsive to the inhibition of HDAC include non-malignant diseases selected from the group comprising

(i) arthropathies and osteopathological conditions such as rheumatoid arthritis, osteoarthrtis, gout, polyarthritis, and psoriatic arthritis;
(ii) systemic lupus erythematosus;
(iii) smooth muscle cell proliferation including vascular proliferative disorders, atherosclerosis and restenosis;
(iv) inflammatory conditions and dermal conditions such as ulcerative colitis, Chrons disease, allergic rhinitis, allergic dermatitis, cystic fibrosis, chronic bronchitis and asthma;
(v) endometriosis, uterine fibroids, endometrial hyperplasia and benign prostate hyperplasia;
(vi) cardiac dysfunction;
(vii) inhibiting immunosuppressive conditions like HIV infections;
(viii) neuropathological disorders like Parkinson disease, Alzheimer disease or polyglutamine related disorders; and
(ix) pathological conditions amenable to treatment by potentiating of endogenous gene expression as well as enhancing transgene expression in gene therapy.

In particular embodiments of the present invention, where gene expression is determined, this is performed via qPCR of a cDNA copy of mRNA transcribed from said gene, wherein said cDNA may be a complete or partial copy of mRNA transcribed from said gene, wherein said mRNA typically comprises more than one exon of said gene in the case of ZFP64 as detailed herein for Transcript variants of ZFP64. In said qPCR, complete or partial copies of said cDNA may be produced (depending on the respective binding sites of the qPCR primers), wherein typically such copies comprise from 50 to 90, particularly from 60 to 80, more particularly from 65 to 75, even more particularly 73 base pairs. In certain cases, more than one transcription variant of said gene (i.e. different mRNAs), based on different combinations of exons of said gene may be present in a sample; in this case, cDNA copies of one or more said transcription variants may be produced, one or more of which may then be processed by qPCR (producing one or more amplification products); the readout to determine gene expression may then also be based on one or more of said amplification products.

In other particular embodiments of the present invention, where gene expression of ZFP64 is determined, this is performed by Taqman® Assay ID Hs00217022_m1 or by a pair of oligonucleotides specifically binding to the target sequence within a cDNA copy of mRNA transcribed from ZFP64 (from which ZFP64 mRNA introns are excluded), having the sequence (Seq ID 1) 5′CACCTCGGAGACCCAGACAATCACAGTTTCAGCTCCAGAA TTTGTTTTTGAACATGGCTATCAAACTTACCTG 3′. Pairs of oligonucleotides which can be used comprise the following, or a combination of Pair 1 Forward primer with Pair 2 Reverse primer or Pair 2 Forward primer with Pair 1 Reverse primer, or such primers can be shorter or longer than these, particularly from 17 to up to 26 bases in length:

Pair 1:
Forward primer
(Seq ID 2)
5′ CACCTCGGAGACCCAGACAA 3′
(20 bases),
Reverse Primer
(Seq ID 3)
5′ CAGGTAAGTTTGATAGCCATGTTCA 3′
(25 bases)
Pair 2:
Forward primer
(Seq ID 4)
5′ CACCTCGGAGACCCAGACA 3′
(19 bases),
Reverse Primer
(Seq ID 5)
5′ CAGGTAAGTTTGATAGCCATGTTC 3′
(24 bases)

Additionally to the primers described above for determination of gene expression of ZFP64 by qPCR method, primers to specifically amplify cDNA sequences of ZFP64 can be used. In general, those probes/primers are short DNA sequences, specifically binding to one or more transcript variants of ZFP64. The length of those primer pairs can comprise 17-25, particularly 19-23, more particularly 20-22 nucleotide bases, and should be designed intron spanning (i.e. forward and reverse primer of a primer pair binding to two separate exons, separated by at least one intron) to avoid amplification of genomic DNA that might be present in the RNA sample. The PCR product obtained by this method can be 50-400, particularly 70-300, more particularly 80-200, more particularly 123 or 198 base pairs in length. PCR products rising from remaining genomic DNA in the RNA sample can theoretically be produced together with amplified cDNA. However such copies of genomic DNA are much longer than amplified cDNA and moreover, a restricted timeframe for the elongation step of the polymerase mediated PCR process prevents elongation of long products (note that even high speed polymerases need 15 seconds for 1000 bases). Thus, the skilled person can easily determine the appropriate duration of amplification steps to avoid formation of excess copies of genetic DNA.

Examples for particular primer pairs for use for amplifying a cDNA copy of mRNA transcribed from ZFP64 in the present invention are:

| | | | Detection |
|---|---|---|---|
| | | | of ZFP64 |
| | | Product | transcript |
| Primer | Sequence | length | variants |
| forward 1 | 5′ ACCTGCCCACG | 198 bp | 1, 3, 4 |
| (Seq ID 6) | GAAAGTAAT 3′ | | |
| reverse 1 | 5′ TATGGGGTTTG | | |
| (Seq ID 7) | TCTCCCGTG 3′ | | |
| forward 2 | 5′ ACCCAGACAAT | 123 bp | 2 |
| (Seq ID 8) | CACAGGTTG 3′ | | |
| reverse 2 | 5′ GCTAAAGCACT | | |
| (Seq ID 9) | TGCCACAGAC 3′ | | |

All of the above primers have an annealing temperature of 58° C.

Transcript variants of ZFP64 by reference number in the NCBI database (http://www.ncbi.nlm.nih.gov/gene/55734)

NM_199427.2; transcript variant 4, mRNA
NM_018197.2, transcript variant 1, mRNA
NM_199426.1, transcript variant 3, mRNA
NM_022088.4, transcript variant 2, mRNA

Further, ZFP64 mRNA level can be determined as well by RNA sequencing. This can be performed by at least two different methods, direct sequencing of mRNA and sequencing of the reversed transcribed mRNA, the cDNA. Methods for these sequencing techniques are well known in the art.

In particular embodiments of the present invention, where gene expression is determined, this is performed by RNA Sequencing. RNA Sequencing, also called “Whole Transcriptome Shotgun Sequencing” (“WTSS”) (RD. Morn, et al. (2008), BioTechniques 45 (1): 81-94), allows to reveal the presence and quantity of a specific RNA from a genome at a given moment in time (Chu Y, Corey D R (2012). Nucleic Acid Ther 22 (4): 271-4). Sequencing-based RNA analysis records the numerical frequency of a given sequence in the sample. Levels of mRNA/cDNA of a gene according to the present invention, in particular ZFP64, are detected (beside other genes) by sequencing the whole cDNA of a cell with a pool of primers spanning the whole transcriptome of a cell.

In particular embodiments of the present invention, where gene expression is determined, this is performed (on the protein level) by Western blot. The western blot (sometimes called the protein immunoblot) is a widely used analytical technique used to detect specific proteins in a sample. It uses gel electrophoresis to separate native proteins by 3-D structure or alternatively denatured proteins by the length of the polypeptide. The proteins are then transferred to a membrane (typically nitrocellulose or PVDF), where they are stained with antibodies specific to the target protein. The gel electrophoresis step is included in Western blot analysis to resolve the issue of cross-reactivity of antibodies. An improved immunoblot method, Zestern analysis (Zhang, Jiandi; Wang, Dan., U.S. Pat. No. 8,293,487), is able to address this issue without the electrophoresis step, thus significantly improving the efficiency of protein analysis. Compared to Western Blot analysis, Zestern analysis adds an elution step is added before the detection step. The immunocomplex formed on the membrane is allowed to access a solution containing an excess amount of competing molecules. The competing molecule can be a synthetic antigen or partial antigen, or multiple repeats of antigen or partial antigen within one molecule. The competition occurs due to the reversibility of the antigen-antibody interaction. Antibodies are liberated by the competing molecule from the membrane into the elution solution. Quantification of the amount of antibodies in the elution solution through a reporter assay gives a reliable indication of the amount of antigen in the protein samples. Therefore, the specific binding of the labeled antibody to the epitope can be quantified by adding a competing antigen. Other related techniques include dot blot analysis, immunohistochemistry where antibodies are used to detect proteins in tissues and cells by immunostaining, and enzyme-linked immunosorbent assay (ELISA). Particular specific antibodies for ZFP64 are e.g.:

Abcam ab66658; Rabbit polyclonal to ZFP64; Immunogen: A region within synthetic peptide DGGQNIAVATTAPPVFSSSSQQELPKQTYSIIQGAAHPALLCPADSIPD (Seq ID 10), corresponding to C terminal amino acids 633-682 of Human ZFP64;
Sigma HPA035112; Rabbit polyclonal; Immunogen sequence SFDTKQPSNLSKHMKKFHGDMVKTEALERKDTGRQSSRQVAKLDAKKSFHCDICDA SFMREDSLRSHKRQHSEYSESKNSDVTVLQFQIEPS (Seq ID11);
ThermoScientific PA5-28546, Rabbit polyclonal; Immunogen: Recombinant fragment corresponding to a region within amino acids 394 and 681 of Human ZFP64

In particular embodiments of the present invention, where gene expression is determined, this is performed (on the protein level) by luminex technology. Luminex technology is a bead-based technology to determine the expression of a given protein in different matrices in an antibody dependent fashion. Therefore, a set of a capture antibody and a detection antibody, both specifically binding to the protein of interest but necessarily to different, non-overlapping epitopes. The capture antibody is coated to a bead, and the detection antibody is labeled (e.g. with a label as described herein), either directly (Phycoerythrin (PE) coupled) or indirectly (biotynilated—detection of this antibody by subsequent streptavidin-PE binding)—or can be detected by a labeled species specific antibody (e.g. labeled anti-rabbit antibody against Zfp64 epitope specific rabbit antibody).

In particular embodiments of the present invention, where gene expression is determined, this is performed (on the protein level) by ELISA. The enzyme-linked immunosorbent assay (ELISA) and enzyme immunoassay are technologies to determine the expression of a given protein in different sample types (e.g. blood plasma, serum, cell lysates etc.) in an antibody dependent fashion. Therefore, a set comprising a capture antibody and a detection antibody is needed, both specifically binding to the protein of interest but necessarily to different, non-overlapping epitopes. The capture antibody is immobilized on a solid phase, e.g. a plastic surface, and the detection antibody is labeled (e.g. with a label as described herein), either directly (e.g. with enzyme converting a substrate into a detectable signal like horse radish peroxidase converting chromogenic substrates like TMB, DAB, ABTS) or indirectly. (biotynilated—detection of this antibody by subsequent streptavidin-enzyme or—streptavidine-fluorescence label binding) or can be detected by a labeled species specific antibody (e.g. labeled anti-rabbit antibody against Zfp64 epitope specific rabbit antibody).

In particular embodiments of the present invention, where gene expression is determined, this is performed at one or more specific time points, e.g. at one or more time points lying directly before administration of an HDAC inhibitor to a patient (also termed 0 h), and from 1 to 10, particularly 1 to 6, more particularly 2 to 5 hours after administration of an HDAC inhibitor to a patient. In particular embodiments of the present invention, where ZFP64 gene expression is determined, this may be performed at one or more specific time points described herein in the context of the clinical trial description (SAPHIRE, SHELTER, SHORE), i.e. directly before administration of an HDAC inhibitor to a patient, about 2 and/or 5 hours after administration of an HDAC inhibitor to a patient.

Typically, if gene expression is measured to determine an effect of an HDAC inhibitor treatment, monitor an HDAC inhibitor treatment, for stratification of a patient or for predicting the probability of a positive outcome, this is performed before, in certain embodiments directly before administration of an HDAC inhibitor to a patient. For stratification of a patient or for predicting the probability of a positive outcome, this is typically performed before start of HDAC inhibitor treatment (i.e. before the first HDAC inhibitor dose is administered to said patient), which may be accompanied by further measurements at later time points, in particular one or more times directly before the start of a treatment cycle, e.g. as described herein in the context of the clinical trial description. To determine an effect of an HDAC inhibitor treatment or monitor an HDAC inhibitor treatment, this may be performed before start of HDAC inhibitor treatment and is typically performed one or more times directly before the start of a treatment cycle, e.g. as described herein in the context of the clinical trial description.

In particular embodiments of the present invention, where ZFP64 gene expression is determined this is performed by contacting the sample with an antibody, particularly an antibody selected from Abcam ab66658; Sigma HPA035112 and ThermoScientific PA5-28546 (described further herein) and measuring binding between a protein expressed by ZFP64 and said antibody. In particular, said binding is measured using a method as described herein, e.g. ELISA, Luminex, etc.

Particular embodiments of the present invention relate to a method of treating a patient in need thereof with an HDAC inhibitor, the method comprising the following steps:
a) providing a sample of said patient, wherein said patient has already received HDAC inhibitor treatment,
b) determining the gene expression and/or the change of the gene expression of at least one gene selected from the group comprising ZFP64, DPP3, CCDC43, HIST2H4A/B, KDELC2, and MICALL1, particularly ZFP64, in said sample,
c) correlating the determined gene expression and/or the change of the gene expression of said at least one gene to an effect of said HDAC inhibitor treatment of step a) in said patient, and
d) administering an HDAC inhibitor to said patient using a dosage of HDAC inhibitor and/or an administration schedule that is determined based on the correlation of step c).

Further particular embodiments of the present invention relate to a method of treating a patient in need thereof with an HDAC inhibitor comprising the following steps:
a) providing a sample of said patient,
b) determining the gene expression of at least one gene selected from the group comprising ZFP64, DPP3, CCDC43, HIST2H4A/B, KDELC2, and MICALL1, particularly ZFP64, in said sample,
c) correlating the determined gene expression of said at least one gene to the probability that an HDAC inhibitor treatment has a beneficial effect on said patient,
d) classifying said patient as a responder to said HDAC inhibitor treatment, based on the probability determined in step c), and
e) administering an HDAC inhibitor to said patient, based on the classification of said patient as a responder.

Further particular embodiments of the present invention relate to a method of stratification of a patient potentially in need of HDAC inhibitor treatment comprising the following steps:
a) providing a sample of said patient, wherein said patient was diagnosed with cancer, particularly hepatocellular carcinoma (HCC), Hodgkin Lymphoma (HL), or colorectal cancer (CRC). More particularly hepatocellular carcinoma (HCC),
b) obtaining a dCt value for the level of gene expression of ZFP64 by subtracting the mean of the Ct values of one or more housekeeping genes from a Ct value determined for ZFP64, and
c) classifying said patient as responder if the dCt value obtained for ZFP64 in step b) is lower than 11.15, particularly lower than 10.02.

Further particular embodiments of the present invention relate to a method of stratification of a patient potentially in need of HDAC inhibitor treatment comprising the following steps:
a) providing a sample of said patient, wherein said patient was diagnosed with cancer, particularly hepatocellular carcinoma (HCC), Hodgkin Lymphoma (HL), or colorectal cancer (CRC). More particularly hepatocellular carcinoma (HCC),
b) obtaining a dCt value for the level of gene expression of ZFP64 by subtracting the mean of the Ct values of one or more housekeeping genes from a Ct value determined for ZFP64, and
c) classifying said patient as eligible for an HDAC inhibitor treatment if the dCt value obtained for ZFP64 in step b) is lower than 11.15, particularly lower than 10.02.

In particular embodiments of the methods of the present invention, said dCT value for gene expression of ZFP64 is obtainable by subtracting the mean of the Ct values of the housekeeping genes 18sRNA, TBP and GAPDH from a Ct value determined for ZFP64.

In particular embodiments of the methods of the present invention, Ct values are determinable by qPCR amplification of a cDNA copy of mRNA expressed by said gene, in particular expressed by ZFP64.

A particular embodiment of the present invention is a kit for determining the gene expression of at least one gene selected from the group comprising ZFP64, DPP3, CCDC43, HIST2H4A/B, KDELC2 and MICALL1, in particular ZFP64, in a sample, wherein the kit comprises a nucleotide probe which (e.g. a PCR primer pair) that binds to a cDNA copy of mRNA expressed by said gene and which is complementary to parts of two exons, in particular a primer pair selected from the primer pairs described herein for use in qPCR of a cDNA copy of mRNA transcribed from said gene, more particularly ZFP64, and

wherein the kit optionally comprises one or more further components selected from the group comprising media, medium components, buffers, buffer components, RNA purification columns, DNA purification columns, dyes, nucleic acids including dNTP mix, enzymes including polymerases, and salts.

Another particular embodiment of the present invention is a k kit for determining the level of at least one protein encoded by a gene selected from the group comprising ZFP64, DPP3, CCDC43, HIST2H4A/B, KDELC2 and MICALL1, in particular ZFP64, in a sample,

wherein the kit comprises a probe which specifically binds to at least one protein encoded by said gene or a domain of said protein, wherein said probe particularly comprises a label as described herein, and/or wherein said probe is an antibody as described herein as particular specific antibody for ZFP64, and/or wherein said probe is immobilized as described herein e.g. in the context of ELISA or Luminex, and wherein the kit optionally comprises one or more further components selected from the group comprising media, medium components, buffers, buffer components, membranes, ELISA plates enzyme substrates, dyes, enzymes including polymerases, and salts.

In the embodiments of the present invention where an antibody or probe which specifically binds to a protein expressed by ZFP64 is applied, said probe or antibody is particularly selected from the particular specific antibodies for ZFP64 as described herein above.

### BRIEF DESCRIPTION OF THE FIGURES

FIG. 1 shows the resminostat dependent, median HDAC enzyme inhibition found in the SAPHIRE clinical trial of the 600 mg dose group in comparison to the 800 mg dose group. The Y axis relates to the enzyme activity in percent in relation to the value at the timepoint at day 1, 0 hours of the HDAC inhibitor treatment, the X axis relates to the treatment cycles, sub-divided into days and hours after start of each respective treatment cycle.

FIG. 2 shows the comparison between the change in gene expression for the genes of the present invention upon HDAC inhibitor administration, measured as expression level (Y axis), as determined in samples of peripheral blood (ex vivo) and selected human cancer cell lines (in vitro) at time points 0, 2 and 5 after administration of an HDAC inhibitor (timepoints shown on the x axis). Hatched columns relate to blood, black columns relate to HepG2 cells, white boxes relate to HT 29 cells. Values are standardized to the value at 0 h.

FIG. 3 shows the comparison between change in gene expression upon HDAC inhibitor administration for housekeeping genes—measured as expression level (Y axis), as determined in samples of peripheral blood (ex vivo) and selected human cancer cell lines (in vitro) at time points 0, 2 and 5 after administration of an HDAC inhibitor (timepoints shown on the x axis). Hatched columns relate to blood, black columns relate to HepG2 cells, white boxes relate to HT 29 cells. Values are standardized to the value at 0 h.

FIG. 4 shows the pharmacodynamic behavior of the CCDC43 gene expression pattern for days 1, 5, 8, and 33, which is in accordance with the HDAC enzyme inhibition displayed in FIG. 1. The values are measured as expression level (Y axis), the X axis relates to the treatment cycles, sub-divided into days and hours after start of each respective treatment cycle.

FIG. 4b shows the ZFP64 down-regulation by Resminostat in blood cells of HCC and HL Patients. Bold lines relate to a daily dose of Resminostat 600 mg (n=14 HCC+16 HL), intermittent lines relate to a daily dose of Resminostat 600 mg+Sorafenib 400 mg (n=19 HCC) and dotted lines relate to a daily dose of Resminostat 800 mg (n=15 HL). Data are shown for relative ZFP64 expression at Day 1 (left panel) and at Day 5 (right panel) dosing.

FIG. 4c shows ZFP64 gene expression data in diverse cancer cell lines after treatment with resminostat (10 μM) at 0, 2, 5, and 24 hours post-treatment.

FIG. 4d shows the treatment of a liver cancer cell line HepG2, as well as whole blood and PBMCs from the same healthy donor with resminostat (5 μM) (“R”) or the combination of resminostat (5 μM) and sorafenib (5 μM) (“R/S”) in terms of the fold change in expression of ZFP64 at 4 h and 2 h hours after administration of said drug or drug combination. For comparison reasons, the siRNA experiment (ZFP64 knockdown) on HepG2 cells is shown. The samples are compared to a DMSO control and the fold change is determined with the exception of the siRNA samples, which are compared to the resminostat sample.

FIG. 5 shows a box plot for the dCt values in blood cells in Hodgkin's Lymphoma from the SAPHIRE clinical study at cycle 1, day 1, hour 0 for ZFP64, with median values (Y axis) 11.08 (progressive disease patients: PD) and 10.67 (stable disease patients: SD), as well as the respective p-value 0.03 (based on Mann-Whitney-test).

FIG. 6 shows ZFP64 baseline expression in blood cells in HCC from the SHELTER clinical study at cycle 1, day 1, hour 0 for ZFP64. Patients are separated by clinical outcome (PD vs. SD).

FIG. 7 shows the biomarker ZFP64 expression in CRC patients from the SHORE clinical study, experiencing PD vs SD. Samples taken at Cycle 1, Day 1, Hour 0 (predose).

FIG. 8 shows boxplots of clinical benefit vs. ZFP64 dCt at baseline comparing 4SC clinical trials (SAPHIRE, SHORE, SHELTER) and healthy volunteers; data for each clinical trial is shown separately, patients are divided into SD and PD group.

FIG. 9 shows boxplots of clinical benefit vs. ZFP64 dCt at baseline comparing 4SC clinical trials data and healthy volunteers; data for all clinical trials (SAPHIRE, SHORE, SHELTER) is consolidated, patients are divided into SD and PD group.

FIG. 10 shows the percentile ranking and splitting for prognostic areas for ZFP64—The upper third indicates a ‘No clinical benefit (PD)’ group with prediction rate=0.78, the bottom third shows a prediction rate=0.69 for the ‘Clinical benefit group (SD)’. The Y axis relates to the percentile, the X axis relates to dCt. In each case, X marks PD patients, dots mark SD patients.

FIG. 11 shows ZFP64 baseline expression in blood cells in HCC from SHELTER clinical study. Patients are separated into the 40^{th} and 60^{th} percentile group with respect to length of overall survival.

FIG. 11b shows ZFP64 baseline expression in blood cells in HCC from SHELTER clinical study. Patients are separated into the 40^{th} and 60^{th} percentile group with respect to length of progression free survival.

FIG. 12 (XXX was 13b) shows SHELTER clinical trial data from HCC patients, ZFP64 expression at baseline vs. overall survival (OS); Kaplan-Meier estimates of overall survival (OS) for the split of ZFP64 relative expression—Baseline ZFP64 expression split at 60th percentile (60% high/40% low). Bold line relates to low relative ZFP64 expression at baseline, intermittent line relates to high relative ZFP64 expression at baseline. Open circles relate to patients alive at the point of data collection.

FIG. 12b shows SHELTER clinical trial data from HCC patients, receiving resminostat (600 mg) or a combination of resminostat (600 mg) and sorafenib (400 mg). ZFP64 expression at baseline vs. overall survival (OS); Kaplan-Meier estimates of overall survival (OS) for the split of ZFP64 relative expression at 75^{th} percentile (75% high/25% low). Bold line relates to high relative ZFP64 expression at baseline, dotted line relates to low relative ZFP64 expression at baseline. Open circles relate to patients alive at the point of data collection, filled circles relate to patients lost to follow-up.

FIG. 13 shows SHELTER clinical trial data—HCC patients receiving resminostat (600 mg); ZFP64 expression at baseline vs. overall survival (OS). The split is calculated by the method described herein, and based only on the specific subgroup of patients receiving resminostat (600 mg). Bold line relates to high relative ZFP64 expression at baseline, dotted line relates to low relative ZFP64 expression at baseline, intermittent line relates to overall Kaplan-Meier plot. Open circles relate to patients alive at the point of data collection, filled circles relate to patients lost to follow-up.

FIG. 14 shows SHELTER clinical trial data—HCC patients receiving resminostat (600 mg) and sorafenib (400 mg)—ZFP64 expression, baseline vs. Overall survival (OS). The split is calculated by the method described herein, and based only on the specific subgroup of patients receiving resminostat (600 mg). Bold line relates to high relative ZFP64 expression at baseline, dotted line relates to low relative ZFP64 expression at baseline, intermittent line relates to overall Kaplan-Meier plot. Open circles relate to patients alive at the point of data collection.

FIG. 15 shows SHELTER data from HCC patients; ZFP64 expression at baseline vs. overall survival (OS). The dashed line relates to high relative ZFP64 expression at baseline, the bold black line relates to low relative ZFP64 expression at baseline, the dashed-dotted line relates to overall Kaplan-Meier plot. Open circles relate to patients alive at the point of data collection. The left panel shows the Resminostat monotherapy arm, the right panel shows the Resminostat/Sorafenib combination arm. The split values are taken from full study cohort (evaluable patients: 6 high/8 low for resminostat, 12 high/6 low for combination arm).

FIG. 16 shows ZFP64 baseline expression in blood cells in Hodgkin Lymphoma from SAPHIRE clinical study. Patients are separated into the 35^{th} and 65^{th} percentile group with respect to length of overall survival.

FIG. 17 shows SAPHIRE data from Hodgkin Lymphoma patients; ZFP64 expression at baseline vs. overall survival (OS)—Baseline ZFP64 expression split at 65th percentile (65% high/35% low). Bold line relates to low relative ZFP64 expression at baseline, intermittent line relates to high relative ZFP64 expression at baseline. Open circles relate to patients alive at the point of data collection.

FIG. 18 shows ZFP64 baseline expression in CRC patients correlated to OS (SHORE clinical trial). Bold line relates to high relative ZFP64 expression, intermittent line relates to low relative ZFP64 expression.

FIG. 19 shows a box plot for the dCt values at cycle 1, day 1, hour 0 for DPP3, with median values 9.15 (PD) and 8.67 (SD), as well as the respective p-value 0.03 (based on Mann-Whitney-test).

FIG. 20, the percentile ranking and splitting for prognostic areas for DPP3 are displayed. The upper third indicates a ‘No clinical benefit (PD)’ group with prediction rate=0.69, the bottom third shows a prediction rate=0.64 for the ‘Clinical benefit group (SD)’. The Y axis relates to the percentile, the X axis relates to dCt. In each case, X marks PD patients, dots mark SD patients.

FIG. 21 displays ZFP64 nuclear protein levels in HepG2 cells which were treated with either 0.1% DMSO (vehicle control) or 5 μM resminostat for 24 h. After isolating nuclear fractions, ZFP64 protein levels were detected by Western Blot analysis. Histone H3 serves as nuclear loading control. ZFP64 protein levels are diminished upon addition of resminostat, whereas Histone H3 levels remain essentially unaltered.

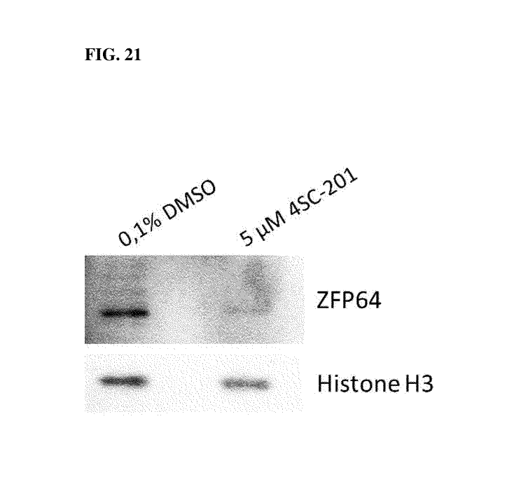

### EXAMPLES

### (E)-3-(1-(4-((dimethylamino)methyl)phenylsulfonyl)-1H-pyrrol-3-yl)-N-hydroxyacrylamide

(INN: resminostat) is a recently developed HDAC inhibitor of the hydroxamate class. The oral administration of resminostat to human subjects was investigated, and its pharmacological behavior and efficacy were determined with the set of biomarkers according to the present invention.

### Example 1

### HDAC Inhibition

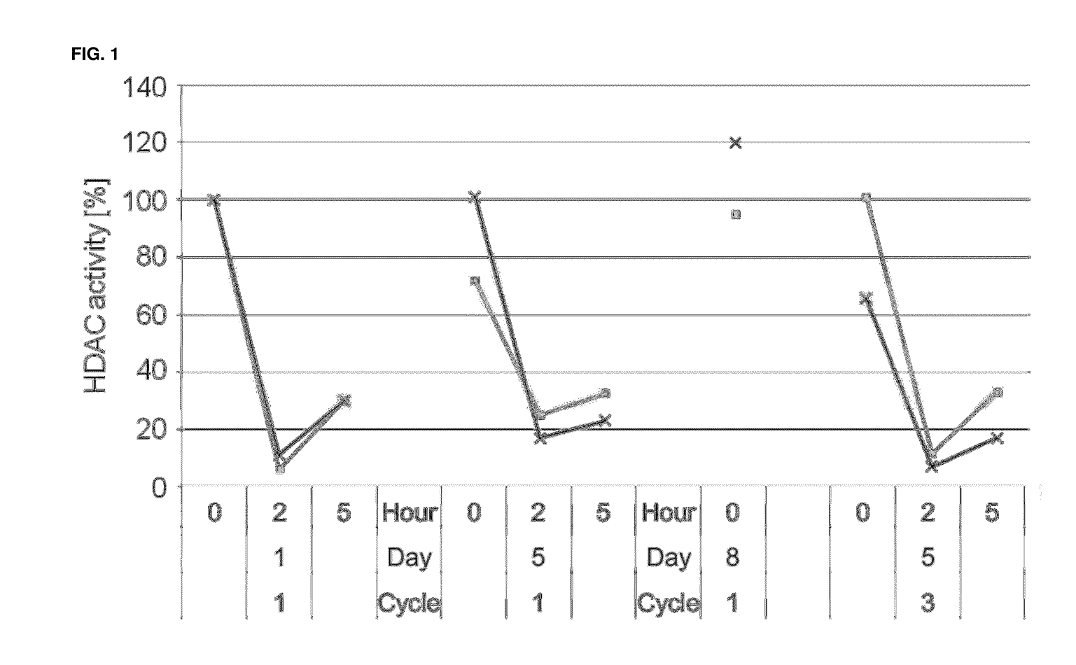

Samples were obtained and gene expressions were determined with the methods as described herein below. Whole blood was incubated for 2 h at 37° C. with fluorescent HDAC substrate Boc-K(Ac)-AMC. After the lysis of erythrocytes remaining cells were stored at −80° C. HDAC activity was determined by fluorimetric analysis using a FLUOstar OPTIMA plate reader, where cells were incubated with a defined developer reagent (containing trypsin and lysis buffer) which leads to cell lysis and generation of a fluorophore from the deacetylated substrate. Finally inhibition of HDAC activity compared to pre-dose levels was calculated. The results are shown in FIG. 1.

Inhibition of HDAC enzyme activity was transiently and time dependent with a maximum inhibition 2 h post-dose corresponding to median peak plasma levels of resminostat between 1.0 h and 1.5 h. HDAC enzyme activity could be inhibited in both dose groups up to a median of 93% 2 h post-dose.

### Example 2

### Correlation Between Gene Expression in Peripheral Blood Cells and Cancer Cells

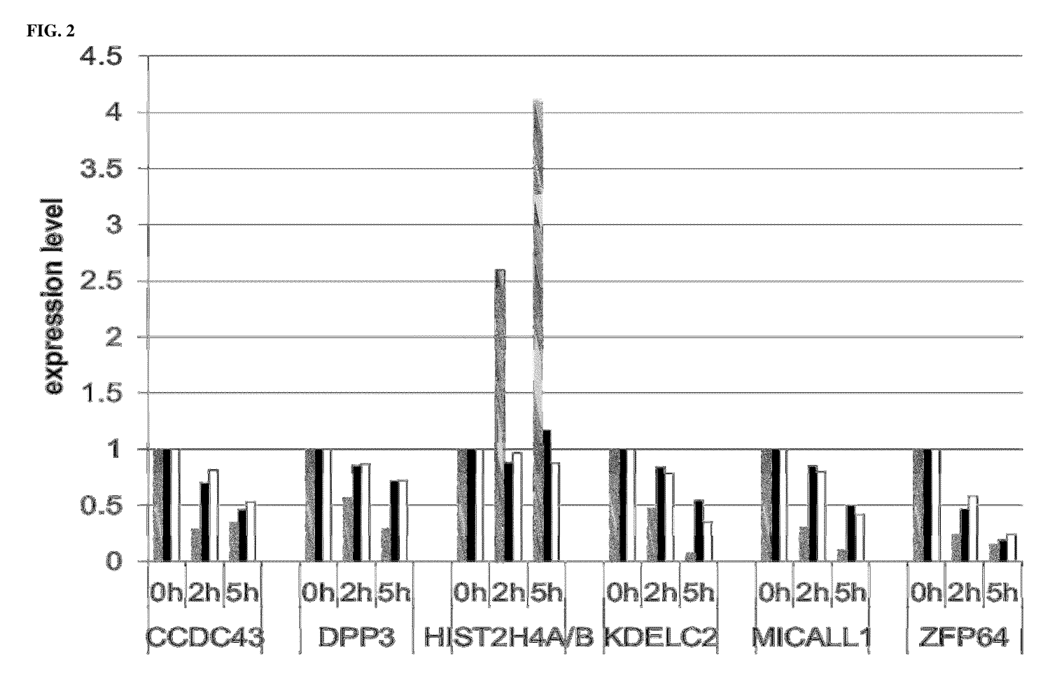

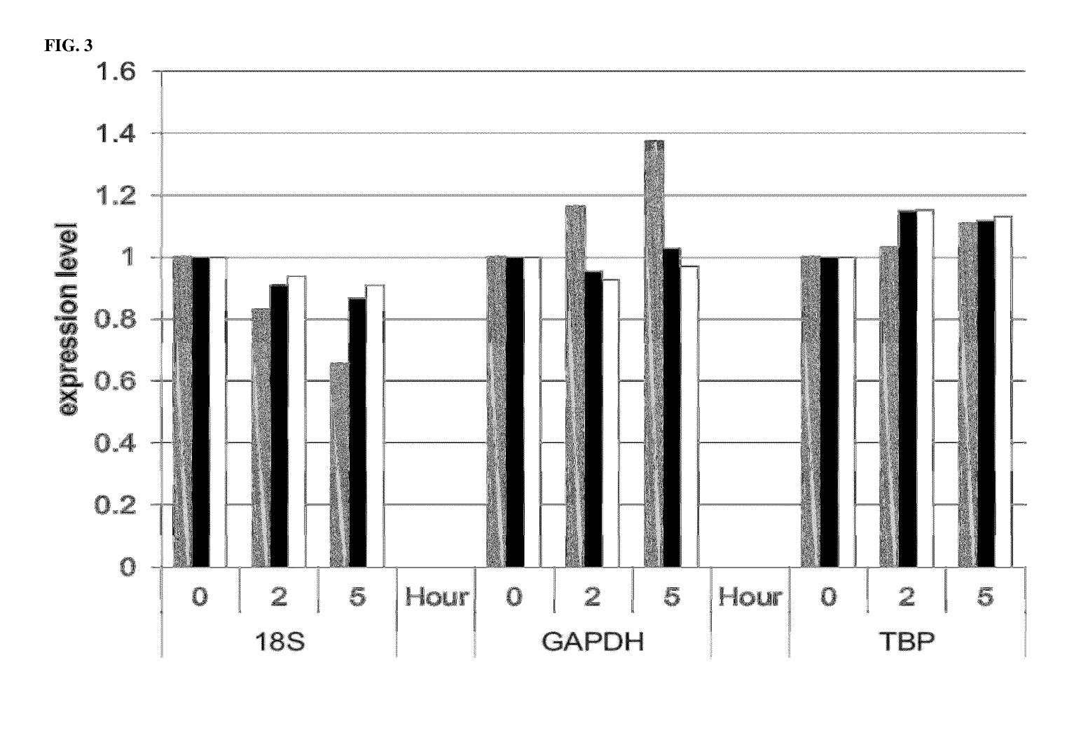

Samples were obtained and gene expressions were determined with the methods as described herein below. The results are shown in below table 3 and FIGS. 2 and 3

The results summarized in FIGS. 2 and 3 show that the biomarkers are regulated in the same manner in blood cells of healthy donors and in cancer cells lines treated with resminostat. The above values represent a mean value which was determined on the basis of 2 biological replicates, for each of which 3 technical replicates were prepared. The results were shown to be reproducible. This indicates that the gene expression of said genes in peripheral blood corresponds to the gene expression in diseased tissue. The level of gene expression determined in peripheral blood samples can be correlated with a certain level of activity of HDAC in the diseased cells, e.g. by applying an appropriate conversion factor or conversion table, which may be determined for each specific disease, and/or patient group.

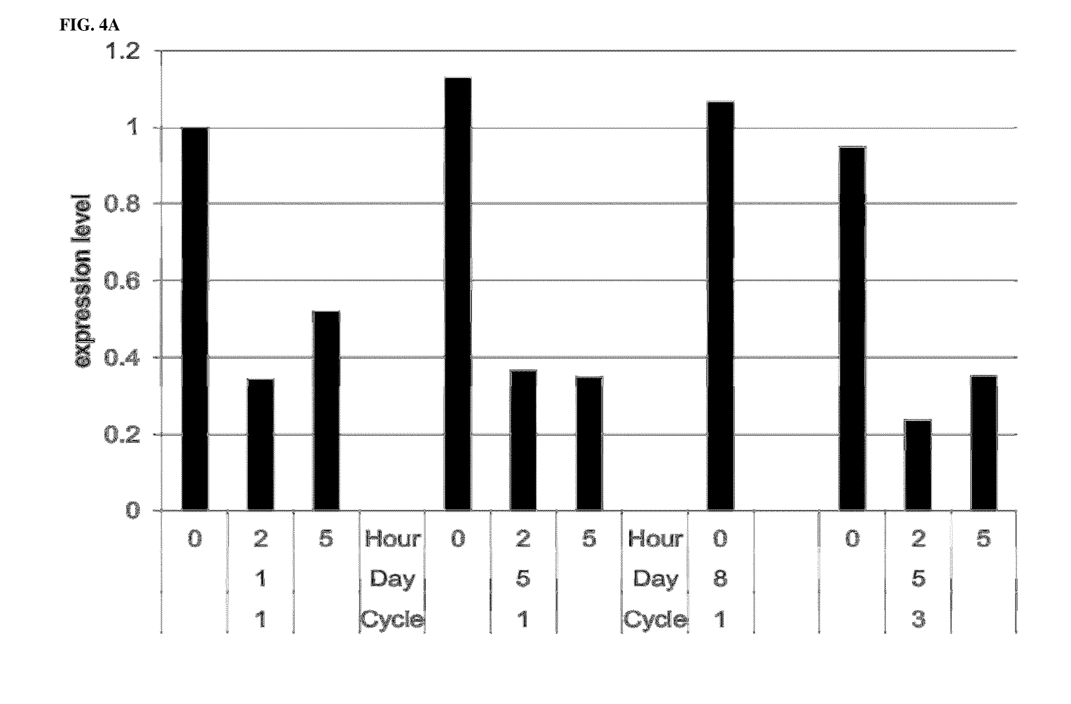

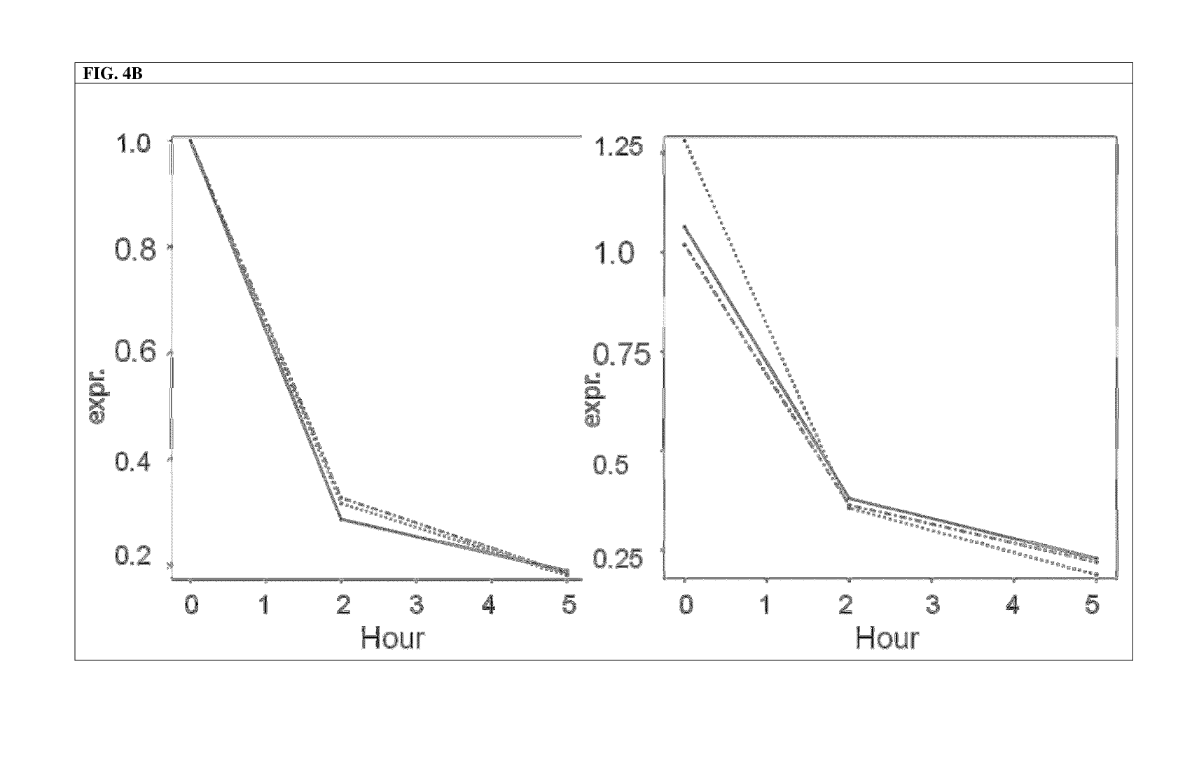

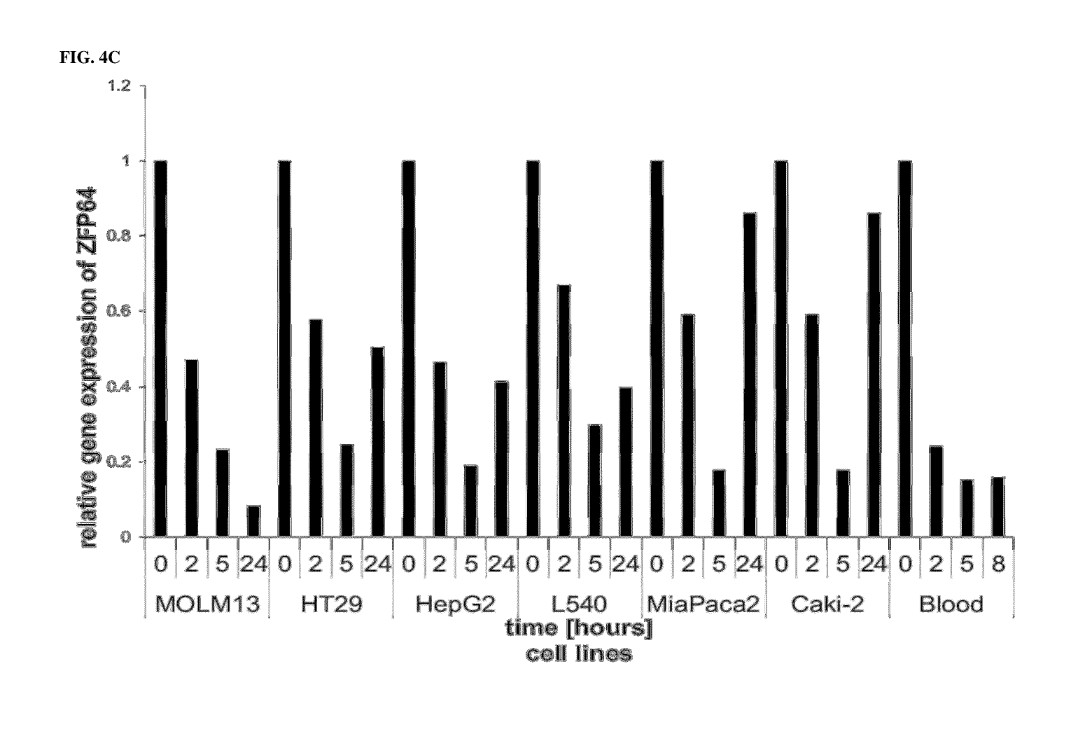

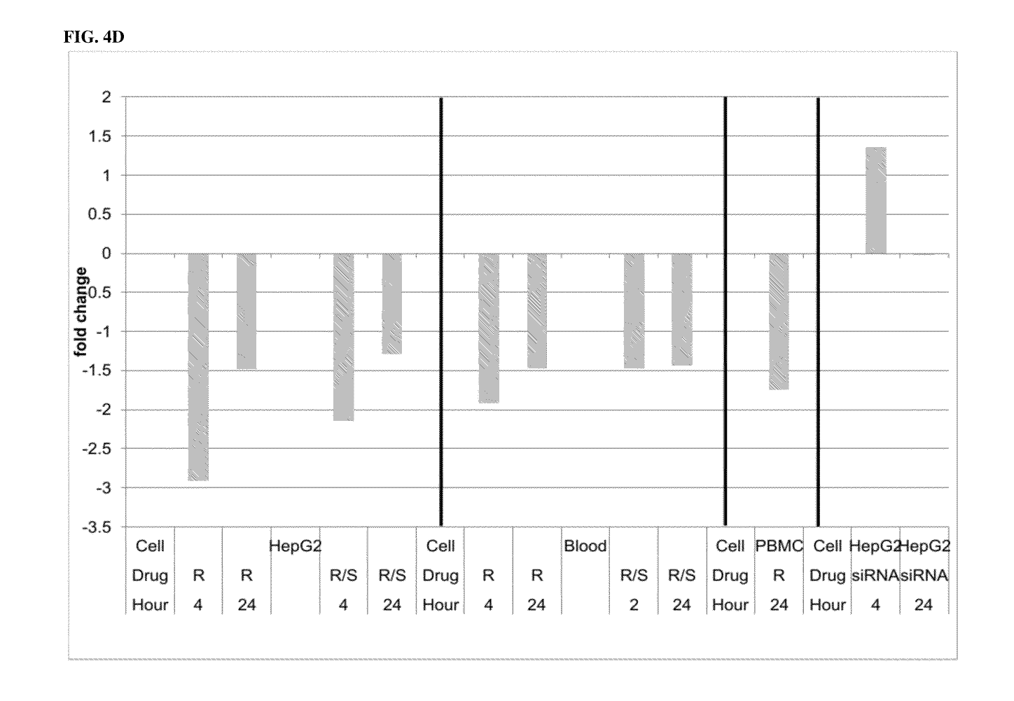

FIG. 4b shows that the HDAC inhibitor Resminostat down-regulates ZFP64 expression in cancer patients, while additional administration of sorafenib does not affect ZFP64 expression.

The same down-regulating effect onto ZFP64 can be seen in FIG. 4c for several cancer cell lines, where the expression levels of ZFP64 at 0, 2, 5, and 24 hours after administration of resminostat are shown. Additionally, in FIG. 4d, the regulation of ZFP64 is displayed in terms of fold change, where a cancer cell line, the whole blood and PBMC from the same healthy donor are compared with each other and are furthermore compared with a siRNA knockdown of ZFP64 in the HepG2 cell line. The same trend of down-regulation can be seen for all samples. The transitional up-regulation of the siRNA sample is due the fact that the expression of the siRNA is compared to the sample with resminostat administration. Typically, down-regulation of target genes by siRNA is delayed compared to small molecule inhibitors.

### Example 3

### Gene Expression Analysis in Samples Obtained from Cancer Patients

HDAC enzyme activity, H4 Histone acetylation and gene expression of a group of genes were measured in the dose groups 100 mg, 200 mg, 400 mg, 600 mg and 800 mg. Each group consisted of 3 patients with different types of tumors. The highest dose group consisted of 6 patients due to a dose-limiting toxicity [DLT] (fatigue and nausea grade 3) of one patient within the first 3 patients in this group. The treatment cycles were as detailed for the SAPHIRE, SHELTER and SHORE clinical trials described herein below.

Drug plasma levels (PK data) were correlated with HDAC enzyme inhibition during drug dose escalation. The analysis showed a prolonged drug effect starting from the 200 mg dose group. As a consequence of these results an additional time point was amended at day 8 in the SAPHIRE trial for the 800 mg dose group. HDAC enzyme activity could be shown to be inhibited in this dose group up to 41% but with a wide range of individual inhibition values.

The mRNAs corresponding to ZFP64, DPP3, CCDC43, HIST2H4A/B, KDELC2 and MICALL1 were extracted via gene chip microarray analysis with the Human Chip U133 v2.0 (Affymetrix Inc., Santa Clara, USA) from 54.675 probe sets and subsequent qPCR. Goal of these experiments was to identify mRNA biomarkers for monitoring HDAC inhibitor effects on transcription of human PBMCs possibly linked to clinical response. Additional selection criteria were up- and down-regulation of the genes of interest with high amplitude and stable expression signals for the HDAC inhibitor.

Three genes were selected as housekeeping genes for the normalization, namely 18sRNA, TBP and GAPDH (Entrez gene IDs are detailed below).

### Clinical Studies Description

The clinical data herein were acquired during clinical trials with (E)-3-(1-(4-((dimethylamino) methyl)phenylsulfonyl)-1H-pyrrol-3-yl)-N-hydroxyacrylamide (INN: Resminostat) mesylate salt, namely the SAPHIRE clinical trial by 4SC AG, Germany (for further reference, see: http://clinicaltrials.gov/show/NCT01037478), the SHELTER clinical trial by 4SC AG, Germany (for further reference, see: http://clinicaltrials.gov/show/NCT00943449), and the SHORE clinical trial by 4SC AG, Germany (for further reference, see: http://clinicaltrials.gov/show/NCT01277406). A short description of the respective study protocols is detailed in the following.

The open label single arm SAPHIRE trial included Hodgkin lymphoma patients who had progressed after prior therapy or were refractory to treatment. Resminostat was administered once daily at 600 mg or 800 mg. Patients were treated in cycles of 5 consecutive days followed by a 9 day treatment-free period (5+9 schedule), constituting one 14 day cycle.

Patients underwent assessment of their disease status by PET/CT. Primary endpoint of the study was the overall objective response rate (ORR) and secondary endpoints included efficacy, safety and tolerability and the analysis of pharmacokinetics of both doses for up to 6 h post dose during the 1st and 3rd treatment cycle. At the same time points, the effect of different doses of Resminostat on pharmacodynamic markers such as HDAC enzyme inhibition and changes in gene expressions of selected target genes was determined in peripheral blood cells.

The SHELTER trial was designed to evaluate safety, PK and efficacy in patients with hepatocellular cancer (HCC) who were refractory to sorafenib to the treatment of Resminostat. Resminostat was explored as monotherapy and in combination with sorafenib. Patients with advanced HCC (BCLC staging B/C) were included in a multi-center, two-arm trial. Radiologic progression under sorafenib first line therapy had to be confirmed by central review (RECIST) prior to study entry. A dose escalation of resminostat (range 200 to 600 mg) combined with Sorafenib (400 or 800 mg) was performed. Arm A investigated the drug combination (resminostat+sorafenib), Arm B the monotherapy of resminostat (600 mg). Primary objective was the progression-free survival rate (PFSR) after 12 weeks (w). Secondary objectives included safety, tolerability, tumor response, PFS, TTP, OS and the analyses of PK and biomarkers (BM), incl. HDAC enzyme inhibition, histone acetylation and gene expressions in peripheral blood.

The SHORE clinical study (4SC-201-3-2010) was designed as a phase I/II study to evaluate safety, tolerability, pharmacokinetics and efficacy of Resminostat in combination with an established second-line chemotherapy regimen (FOLFIRI) for patients with k-ras mutated advanced colorectal carcinoma (CRC).

Main inclusion criteria included: age ≧18 years, histological or cytological confirmed advanced or metastasized k-ras mutated colorectal cancer. Patients must have previously received treatment with 5-FU and be eligible for second-line treatment with FOLFIRI. For the Phase I part, k-ras wildtype status and subsequent treatment lines were also allowed for inclusion.

The primary objective of the Phase I part was to determine the MTD of resminostat in combination with FOLFIRI by investigating safety, tolerability and pharmacokinetics of said combination. Secondary objectives were to assess PFSR after 8 weeks (4 cycles) and every following 8 weeks, PFS, TTP, number of objective responses, OS and DOR. Further, biomarkers were investigated including HDAC enzyme inhibition, histone acetylation, gene expression analysis, protein biomarkers and tumor markers such as CA 19-9 and CEA. At the filing date of the present application, no patients were recruited after phase I, and the study was marked as “active, not recruiting on http://clinicaltrials.gov.

During the Phase I part, cohorts of 3-6 patients received escalating doses of resminostat from 200-800 mg per day, combined with a standard regimen of FOLFIRI treatment until determination of the MTD of the combination. In each 14-day treatment cycle, patients were dosed on 5 consecutive days (Days 1-5) with resminostat followed by a 9-day rest period (Days 6-14). On Days 3 and 4, compounds of the FOLFIRI regimen were administered.

As of the filing date of this patent application, 17 patients were enrolled in the Phase I part: 3 on each of the dose levels 200 mg, 400 mg and 600 mg resminostat plus FOLFIRI, and 8 patients in the dose level 400 mg Resminostat BID plus FOLFIRI.

### Material and Methods

### Samples

2.5 ml whole blood from patients was collected in PAXgene™ tubes (BD Biosciences) that contain chemicals to lyse the erythrocytes, stabilize RNA from degradation by RNases and minimize ex vivo changes in gene expression. The samples were incubated at 20° C. to 25° C. for 2 hours and then frozen at −20° C. until purification of total RNA.

### Preferred Protocols for Sample Collection are Described in the Following:

All procedures were done on ice. The following steps were performed:

Collect venous blood samples (2 ml) in labeled K-EDTA vacutainers (e.g. Monovettes®). The time point “0 h” refers to the time just before dosing (predose sample).

Cycle 1, day 1: 0 h (pre-dose); 2 h; 5 h

Cycle 1, day 5: 0 h (pre-dose); 2 h; 5 h

Cycle 1, day 8: 0 h (pre-dose)

Cycle 3, day 5: 0 h (pre-dose); 2 h; 5 h

The accepted time deviation is, unless stated otherwise: +/−10 min.

### PBMC Isolation:

### Materials Needed

Labelled Leucosep™ tubes (Greiner bio-one) (to be stored at +4-+8° C. and in the dark until use, to be warmed to RT before use)
Labelled 15 ml tubes (e.g. PP tubes, e.g. Falcon® tubes)
Labelled 2 ml tubes (e.g. PP tubes)
Erythrocyte lysis buffer (Qiagen 79217) (to be stored at RT until use)
PBS (to be stored at RT until use)
RIPA buffer (Thermo Scientific 89900) (to be stored at 4° C. until use)
Protease Inhibitor Cocktail stock, provided as 10 μL aliquot (Thermo Scientific 87785) (to be stored at −80° C. until use)

### Sample Processing

1) Transfer 7 ml citrate blood (blood sample treated with citrate by commonly known procedures to inhibit coagulation) of a treatment to a ready-to-use Leucosep™ tube.
2) Centrifuge for 15 min at 800 g, without brake at RT.
3) Remove approx. 2 ml of plasma supernatant (contains thrombocytes) leaving up to 5 mm over the PBMC interphase.
4) Transfer the complete rest of the supernatant above the porous barrier by pipetting to a labelled 15 ml PP (e.g. Falcon®) tube suitable for centrifuging.
5) Add 10 ml of PBS (RT) and mix.
6) Centrifuge for 7 min at 400 g with brake at RT.
7) Remove supernatant except for a resting volume of approx. 0.5 ml PBS and resuspend cells.
8) Add 1.5 ml Erylysis (erythrocyte lysis)-Buffer (Qiagen 79217) and incubate for 4 min at RT for lysis.
9) Add 10 ml PBS to stop Erylysis (erythrocyte lysis).
10) Centrifuge for 7 min at 400 g at RT.
11) Remove supernatant and resuspend cells in 14 ml PBS.
12) Centrifuge for 7 min at 400 g at RT.
13) Remove supernatant and resuspend cells in 4.5 ml PBS.
14) Transfer three equal aliquots (3×1.5 ml) from the cell suspension into labelled 2 ml tubes.
15) Centrifuge for 7 min at 400 g at RT.
16) Remove supernatant completely according to the following procedure: tilt the tube to a 45 degree angle with the cell pellet facing upward and remove the liquid opposite to the cell pellet using a gel loader tip. Place on ice.
17) Transfer 1000 μL of cold RIPA-buffer to one tube of Protease Inhibitor Cocktail stock and mix.
18) Transfer 50 μL of this Protease Inhibitor Cocktail solution to each of the three aliquotted cell pellets and mix briefly.
19) Store pellets immediately at −80° C.

### Cellular HDAC Enzyme Activity Assay Protocol

### Materials Needed

Labelled 15 ml tubes (e.g. PP tubes, e.g. Falcon® tubes)
Labelled 2 ml tubes (e.g. PP tubes)
Erythrocyte Lysis buffer (Qiagen 79217) (stored at RT until use)
40 mM Boc-K(Ac) AMC Stock in DMSO (Bachem: 1-1875) (stored at −80° C. until use)

### Sample Processing

1) Add 1 ml citrate blood into a 15 ml labelled tube.
2) Add 5 μl 40 mM Boc-K(Ac) AMC to 1 ml citrate blood (final concentration: 200 μM Boc-K(Ac) AMC).
3) Incubate for 2 h in an incubator (37° C.).
4) Add 5 fold volume (5 ml) of 4° C. cold erythrocyte lysis buffer (EL buffer, Qiagen 79217).
5) Mix briefly on a shaker.
6) Incubate on ice for at least 15 min; mix two times in between on a plate shaker.
7) Add 7 ml of 4° C. EL buffer.
8) Centrifuge for 10 min at 400 g at 4° C. and remove the supernatant.
9) Add two fold volume (2 ml) 4° C. EL buffer and mix briefly.
10) Centrifuge for 10 min at 400 g at 4° C. and remove the supernatant.
11) Add two fold volume (2 ml) 4° C. EL buffer and mix briefly.
12) Take the 2 ml tubes labelled with the correct time point to sample.
13) Write the patient number onto the tube label.
14) Prepare two 1 ml aliquots (mix again with a pipette before aliquoting).
15) Centrifuge for 10 min at 400 g at +4° C. and remove the supernatant completely according to the following procedure: tilt the tube to a 45 degree angle with the cell pellet facing upward and remove the liquid opposite to the cell pellet using a gel loader tip.
16) Freeze samples at −80° C.
   RNA Isolation from Cell Lines and PBMC

Samples e.g. cells are disrupted, e.g. mechanically (e.g. sonification) or chemically (e.g. with a detergent, such as Sodium Dodecyl Sulfonate, Guanidine Isothiocyanate), to get access to the RNA. To extract the RNA form the cell lysate containing RNA, DNA, proteins and other entities, different methods can be used, e.g. extraction methods using organics (e.g. phenol/chloroform), filter-based spin basket formats (e.g. glass fiber, derivatized silica, or ion exchange membranes, to which nucleic acids bind), magnetic particle methods (particles with paramagnetic core and surrounding shell modified to bind to nucleic acids like silica), and direct lysis methods (e.g. with lysis buffer formulations that disrupt samples and stabilize nucleic acids).

To determine RNA expression levels of ZFP64 in different cancer cell lines and PBMC, the cell membranes were chemically disrupted with a buffer containing guanidine isothiocycanate, which lyses the cells and supports the binding of RNA to a silica membrane and RNA was extracted via a filter-based spin column.

### PaxGene Protocol

1) 2 labelled PaxGene® tubes (Qiagen 79217) (to be stored at RT until use) are taken per time point and patient.
2) After addition of 2 ml of blood mix the tubes by inverting it 10 times.
3) Incubate the tubes filled with blood for 2 hours at RT
4) Transfer the tubes to a −20° C. freezer
5) Store the blood containing tube at −20° C. at least for 24 h before shipment

As used herein, RT means room temperature, which is typically in the range of 21-25° C. Preferred methods of biomarker measurements are described in the European Patent Application No. 12179187.5

RNA Isolation from Whole Blood Samples

Total RNA was isolated from the whole blood samples stabilized in PAXgene™ buffer using a spin column based technique suitable for whole blood samples (like PAXgene™ Blood miRNA Kit or PAXgene™ Blood RNA Kit from Qiagen) according to the manufacturer's instructions. The purification began with a centrifugation step to pellet nucleic acids in the (PAXgene™ Blood RNA) tube. The resuspended pellet was incubated in buffers, optimized for maintenance of RNA stability together with proteinase K to bring about protein digestion. An additional centrifugation through the PAXgene™ Shredder spin column was carried out to homogenize the cell lysate and remove residual cell debris, and the supernatant of the flow-through fraction was transferred to a fresh microcentrifuge tube. Ethanol was added to adjust binding conditions, and the lysate was applied to a PAXgene™ RNA spin column. During a brief centrifugation, RNA was selectively bound to the PAXgene™ silica membrane as contaminants pass through. Remaining contaminants were removed in several efficient wash steps. Between the first and second wash steps, the membrane was treated with DNase I, as described herein below in 1.3 to remove trace amounts of bound DNA. After the wash steps, RNA was eluted in nuclease free water and heat-denatured.

### Additional DNase Digestion and Clean-Up

To enhance the purity of the RNA, an additional in-solution DNase digestion was carried out, using the RNase-free DNase Set (Qiagen). Briefly, the eluted RNA was incubated for 10 minutes at room temperature with 6.81 Kunitz units RNase free DNase I. Kunitz units are the commonly used units for measuring DNase I, defined as the amount of DNase I that causes an increase in A260 of 0.001 per minute per milliliter at 25° C., pH 5.0, with highly polymerized DNA as the substrate (Kunitz, M. [1950] J. Gen. Physiol. 33, 349 and 363). The samples were then purified using a spin column based technique (RNeasy® Mini Kit from Qiagen).

### Determination of RNA Concentration and Purity

An aliquot of each total RNA sample was used to determine RNA concentration and purity on a spectral photometer (NanoDrop® ND-1000 spectral photometer (peqlab).

### RNA Integrity Control

RNA integrity was tested but is not essential, because for a given sample, RNA quality is comparable over all measured RNAs in the sample, thus leveling any potential aberrance. All samples were analyzed on the 2100 Bioanalyzer (Agilent Technologies) using RNA 6000 Nano or RNA 6000 Pico LabChip Kits (Agilent Technologies), depending on the total RNA concentration.

The 2100 Bioanalyzer allows for analysis of total RNA samples by capillary electrophoresis. The RNA is separated according to fragment size, and results are returned as electropherograms and virtual gel images.

An index for RNA quality, the so-called RIN (RNA integrity number) is derived from the electrophoretic profile. The RIN scale ranges from 1 to 10. A RIN of 10 denotes an excellent RNA quality, while a RIN of 1 indicates massive degradation. For RIN calculation, the algorithm does not rely on the 28S/18S-rRNA ratio alone, but takes into account the entire electrophoretic profile (e.g. the fraction of short degraded RNA species, e.g. about 20 nucleobases in length) (Schroeder et al., 2006, BMC Molecular Biology 7:3).

### Reverse Transcription

The High-Capacity cDNA Reverse Transcription Kit (Applied Biosystems) was used for reverse transcription of total RNA into single stranded cDNA with the aid of random hexamer primers according to the manufacturer's instructions. Briefly, for the reaction mix total RNA was mixed with random primers in excess, 4 mM dNTP Mix, 2.5 U/reaction, reverse transcriptase and nuclease free water. The reverse transcription took place in a thermal cycler under the following conditions: 25° C. for 10 min, 37° C. for 120 min, 85° C. for 5 min, followed by 4° C. for cooling down the reaction. The reverse transcription can be done in principle as well with other reverse transcriptases, primers and temperature protocols. Whenever possible, 250 ng total RNA were reverse transcribed if not available, lower amounts were used.

### Quantitative Real-Time PCR on Custom TaqMan® Arrays

Custom TaqMan® Arrays (Applied Biosystems) in Format 16 were designed for the gene expression analysis of biomarkers of the present invention and housekeeping genes as outlined in table 4. These arrays allow for qPCR analysis of 8 samples per card. Of course, all reactions can be done as well by conventional qPCR analysis with specific primers.

TABLE 4
The target and housekeeping gene assays contained
on the Custom TaqMan ® Arrays

| | | | | TaqMan ® |
|---|---|---|---|---|
| | | Entrez | | Assay ID |
| Gene | | Gene ID | | (Applied |
| Symbol | Gene Name [human] | [human] | Category | Biosystems) |
| 18S | Eukaryotic 18S rRNA | n.a. | Housekeeping | Hs99999901_s1 |
| | | | gene | |
| GAPDH | glyceraldehyde-3- | 567 | Housekeeping | Hs99999905_m1 |
| | phosphate dehydrogenase | | gene | |
| TBP | TATA box binding | 2597 | Housekeeping | Hs99999910_m1 |
| | protein | | gene | |
| CCDC43 | coiled-coil domain | 124808 | Target gene | Hs00327475_m1 |
| | containing 43 | | | |
| DPP3 | dipeptidyl-peptidase 3 | 10072 | Target gene | Hs00366603_m1 |
| HIST2H4A | histone cluster 2, H4a and | 8370, | Target gene | Hs00269118_s1 |
| HIST2H4B | histone cluster 2, H4b | 554313 | | |
| KDELC2 | KDEL (Lys-Asp-Glu- | 143888 | Target gene | Hs00794053_m1 |
| | Leu) containing 2 | | | |
| MICALL1 | MICAL-like 1 | 85377 | Target gene | Hs00411017_m1 |
| ZFP64 | zinc finger protein 64 | 55734 | Target gene | Hs00217022_m1 |
| | homolog (mouse) | | | |

The microfluidic cards were loaded with TaqMan® Gene Expression Master Mix (Applied Biosystems) and 200 ng cDNA per port, or less if no more available.

The reaction mix was transferred into the reaction chambers by centrifuging twice for 1 minute each at 330 g and 4° C. After sealing, the microfluidic cards were run on an AB7900HT instrument (Applied Biosystems). The software SDS 2.4 (Applied Biosystems) was employed for instrument control, data acquisition and raw data analysis. The plates were run in Relative Quantification (ΔΔCt) mode, and the following temperature profile was used:

50° C./2:00 min-94.5° C./10:00 min-[97° C./0:30 min-59.7° C./1:00 min] for 40 cycles.

### Nuclear Protein Extraction and Western Blot Analysis

| | |
|---|---|
| HepG2 | hepatocellular carcinoma cells |
| 75 cm² cell culture flasks | Greiner Bio-one, Cat. 658170 |
| Nuclear Extraction Kit | Abnova, Cat. KA1346, Lot 0448735 |
| NP-40 Alternative (10%) | Protein Grade Detergent, Calbiochem, Cat. 492018 |
| CriterionTM Precast Gel | 12% Bis-Tris, 18 Well Comb, 30 μl, Bio-RAD |
| | Laboratories, Cat. 345-0118 |
| Tricine Sample Buffer | Bio-RAD Laboratories, Cat. 161-0739 (before use, add 5 μl |
| | β-Mercaptoethanol to 250 μl sample buffer)Precision Plus |
| | ProteinTM Dual Color Standards Bio-RAD |
| | Laboratories, Cat. 161-0374 |
| Biotinylated Protein Ladder | Cell Signaling, Cat. 7727 |
| XT MOPS (20x) Buffer | Bio-RAD Laboratories, Cat. 1161-0788 |
| Tris/CAPS Buffer (10x) | Bio-RAD Laboratories, Cat. 161-0778 |
| Immun-Blot PVDF Membrane (0.2 μm) | Bio-RAD Laboratories, Cat. 162-0177 |
| Filter Paper Criterion Size | Bio-RAD Laboratories, Cat. 1703967 |
| SuperBlock T20 (PBS) Blocking Buffer | Thermo Scientific, Cat. 37516 |
| Detection Reagent 1 Peroxide Solution | Thermo Scientific, Cat. 1859701 |
| Detection Reagent 2 Luminol Enhancer Solution | Thermo Scientific, Cat. 1859698 |
| Blotting Buffer | 50 ml 10x Tris/Caps Buffer + 2.5 ml 20% SDS |
| | add 500 ml with H₂O |
| Washing Buffer (PBS + 0.05% Tween20) | 25 ml PBS + 25 ml 0.1% Tween20 in PBS |
| Gel Doc 2000 | Bio-RAD Laboratories |

### Primary Antibodies:

| Target Protein | Cat. No^{o} | Lot No^{o} | Dilution/Conc. | Source |
|---|---|---|---|---|
| ZFP64 | ab66658 | GR-124850-1 | 1.25 μg/ml | Abcam |
| Histone H3 | ab1791 | GR153323-2 | 1/2000 | Abcam |

### Secondary Antibodies:

| Antibody | Cat. No^{o} | Dilution/Conc. | Source |
|---|---|---|---|
| Anti-Biotin-HRP | 7075 | 1/1000 | Cell Signaling |
| Histone H3 | 111-035-003 | 1/50,000 | Jackson |

4 h upon seeding, HepG2 cells were treated with either 0.1% DMSO or 5 μM resminostat.

24 h upon resminostat treatment, cells were washed once with PBS before scraping and harvesting in cold PBS and transferring the cells into pre-chilled 15 ml falcon tubes.

For preparing cytoplasmic and nuclear extracts, first cell swelling is caused by resuspension in hypotonic buffer. Afterwards a detergent (such as Nonident P-40) is added, which breaks the cell membranes and thereby permits access to the cytoplasmic fraction. Cellular fractionation is performed by centrifugation and removing of the cytoplasmic extract. Subsequently, the nuclei are lysed using a nuclear extraction buffer.

Cytoplasmic Extract Buffer: 10 mM HEPES, 60 mM KCl, 1 mM EDTA, 1 mM DTT, 1 mM PMSF, adjusted to pH 7.6; Detergent: 0.075% (v/v) NP-40; Nuclear Extraction Buffer: 20 mM Tris Cl, 420 mM NaCl, 1.5 mM MgCl2, 0.2 mM EDTA, 1 mM PSMF, 25% (v/v) glycerol, adjusted to pH 8.0

### Preparation of Buffers Using the Abnova Nuclear Extraction Kit:

| | |
|---|---|
| | 60 mm cell |
| Reaction vessel | culture plate |

PBS/Phosphatase Inhibitor Solution (1X)

| | | |
|---|---|---|
| Nuclear Extraction PBS (10X) | 0.6 | ml |
| Distilled Water | 5.28 | ml |
| Nuclear Extraction Phosphatase Inhibitors (50X) | 0.12 | ml |
| Total Volume | 6 | ml |

Hypotonic Buffer (1X)

| | | |
|---|---|---|
| Nuclear Extraction Hypotonic Buffer (10X) | 25 | μl |
| Nuclear Extraction Phosphatase Inhibitors (50X) | 5 | μl |
| Nuclear Extraction Protease Inhibitors (100X) | 2.5 | μl |
| Distilled Water | 217.5 | μl |
| Total Volume | 250 | μl |

Extraction Buffer (1X)

| | | |
|---|---|---|
| Nuclear Extraction Buffer (2X) | 25 | μl |
| Nuclear Extraction Protease Inhibitors (100X) | 0.5 | μl |
| Nuclear Extraction Phosphatase Inhibitors (50X) | 1 | μl |
| DTT (10 mM) | 5 | μl |
| Distilled Water | 18.5 | μl |
| Total Volume | 50 | μl |

The nuclear extraction was performed as described by the manufacturer (Abnova, Cat.# KA1346) as follows:
centrifugation of suspended cells at 300×g, 5 min, 4° C.
supernatant is discarded, resuspension of pellet in 3 ml ice-cold PBS/Phosphatase inhibitor solution
centrifugation 300×g, 5 min, 4° C.
supernatant discarded, resuspension of pellet in 3 ml ice-cold PBS/Phosphatase inhibitor solution
centrifugation 300×g, 5 min, 4° C.
supernatant discarded, 250 μl ice-cold Hypotonic buffer added, mixed and resuspended pellet transferred into pre-chilled 1.5 ml tubes
incubation on ice for 15 min
50 μl 10% Nonident P-40 added, gently mixed by pipetting
centrifugation 30 sec, 4° C.
supernatant (contains cytosolic fraction) transferred into new tube
pellet in 50 μl ice-cold Nuclear extraction buffer resuspended, vortexed 15 sec, rocking on ice for 15 min, vortexed 30 sec, rocking on ice for 15 min
centrifugation 14000×g, 10 min, 4° C.
supernatant (contains nuclear fraction) transferred into new tube
cytosolic and nuclear samples were stored at −80° C.

From each nuclear sample 4.4 μg protein (measured via BCA protein assay) were added to the same amount of Tricine buffer (supplemented with B-Mercaptoethanol), heated at 95° C. for 5 min and then placed on ice. The sample—Tricine buffer mixes were applied into a well of a precast 12% Bis-Tris gel. The gel was run at 80-110 V.

Blotting papers and PVDF membrane (shortly activated in methanol) were soaked in Blotting buffer and piled up in the blotting gadget. The gel was blotted onto a membrane with constant 180 mA for 45 min. Afterwards the membrane was placed in a plastic box containing SuperBlock T20 (PBS) Blocking Buffer and incubated shaking at room temperature for 2 h. After cutting the membrane to the appropriate sizes, these membrane pieces were incubated overnight at 4° C. in 10 ml 1/10 diluted SuperBlock T20 Blocking Buffer containing the respective primary antibody, diluted as described in the antibody table.

The next day, after washing four times with Washing Buffer, membranes were incubated for 1 h with the corresponding horseradish peroxidase-conjugated anti-rabbit IgG secondary antibody at a dilution of 1/50.000 and anti-Biotin at a dilution of 1/1.000 (diluted in 10 ml 1/10 diluted SuperBlock T20 Blocking Buffer). After washing four times with Washing Buffer, HRP signals were detected using enhanced chemoluminescence and exposed to X-ray films. Subsequently, the films were scanned using the Gel Doc 2000 and the corresponding signals were quantified with the “Quantity One” Software (BioRad).

### Data Analysis

### Analysis Settings

For downstream analysis, the real-time PCR runs of the 384-well microfluidic cards were loaded into the software RQ Manager 1.2.1 (Applied Biosystems). Separate studies (*.sdm files) were generated for each of the tested patients.

For each well, Ct values (Cycle threshold), i.e. the cycle number where the amplification curve clearly exceeds the background and the exponential curve is in the linear phase, were calculated in the software RQ Manager 1.2.1. Whenever possible, the instrument's setting “Automatic Ct” was used, and all manual settings were defined based on inspection of the amplification plots. The resulting Ct values were then averaged within the triplicate measurements of each sample/assay combination to generate Ct Avg values. The associated variation (Ct SD, which is equivalent to the standard error of the mean of Ct Avg) was calculated using the algorithm of the software RQ Manager 1.2.1.

### Quality Filtering (Technical Uniformity)

The raw signals of each well, namely the 6-FAM (6-Carboxyfluorescein) signal and the ROX (6-Carboxyl-X-Rhodamine) signal (passive reference dye contained in the master mix) were closely inspected. Whenever irregularities (such as unexpected buckles or shifts in the curves) were observed, the effects on the processed signals and the resulting amplification plots were checked. If necessary, wells with irregular raw signals were omitted from downstream analysis.

Furthermore, the uniformity of the triplicate measurements of each sample/assay combination was evaluated based on the Ct SD values. The quality filter Ct SD≦0.25 was applied, meaning that whenever the Ct SD value was found to be >0.25, the amplification plots of the triplicate wells were closely inspected. Whenever an outlier well, i.e. dCt>1 compared to the other replicates, was found, it was excluded from further analysis. Such an outlier might be caused by insufficient filling of a reaction chamber, irregular processes during PCR, e.g. bursting of tiny air bubbles or a production error caused by the manufacturer.

If target gene expression is low (e.g. Ct>32) and the starting number of molecules is very limited (between 1,000-10,000 copies, dependent of the molecule and the qPCR assay), stochastic effects exert a dominating influence on the PCR amplification process, resulting in variable Ct values. Especially for Ct values >32, low reproducibility of technical replicate measurements is observed. For this reason, triplicate wells with high Ct values and a Ct SD>0.25 were not excluded from analysis. However, their results must be interpreted with caution.

In cases where Ct values were <32, but Ct SD values were >0.25 and no clear outlier could be identified, all three data points were used for downstream calculations.

### Calculation of Relative Expression Levels

The ΔΔCt method was applied to calculate relative expression levels of the target (biomarker) transcripts. This method standardizes the gene expression of a target gene to the expression of one or more housekeeping genes and then relates it to the gene expressions of target and housekeeping genes in a calibrator (reference) sample or group.

### Basic Concept of the ΔΔCt Method:

In a first step, the Ct values of the triplicate measurements of a gene in sample A are averaged to create an Avg Ct value. The difference between the Avg Ct value of the target gene and the Avg Ct value of the housekeeping gene is calculated (ΔCt value). Subsequently, the difference of the ΔCt value of sample A and the ΔCt value of the calibrator sample is calculated (ΔΔCt value).

Based on the equation 2^{-ΔΔCt}, the RQ value (Relative Quantity) is determined, which indicates the relative expression of a target gene in sample A as compared to the calibrator sample. The calibrator sample obtains an RQ value of 1.000 for all target genes. The RQ value is equivalent to an X-Fold Change value.

The basic ΔΔCt calculation was performed using the RQ Manager software. Results of the different Relative Quantification studies were calculated using 18S as the housekeeping gene and the first sample of each patient (cycle 1, day 1, hour 0) as calibrator sample.

Modification of the ΔΔCt method to implement several housekeeping genes:

The R/Bioconductor package ddCt (v1.5.0, http://www.bioconductor.org/packages/bioc/html/ddCt.html; Authors: Jitao David Zhang, Rudolf Biczok and Markus Ruschhaupt) was implemented for advanced calculation of relative expression levels. This tool offers an approach to combine several housekeeping genes (and also, if needed, to integrate several samples into the calibrator (reference) group).

The following calculation steps are carried out by the ddCt script:

The mean of the technical replicates for each gene-sample combination is calculated and is called MeanCt. (In the original output tables from ddCt, this column is simply called ‘Ct’. In order to better discern it from the Ct values of individual technical replicates, it was renamed as “MeanCt” after ddCt analysis).

The mean of the MeanCt values of all housekeeping genes is calculated for each sample.

The mean of the MeanCt values of all housekeeping genes is subtracted from the corresponding MeanCt value of a gene Gene1. The resulting value is called dCt.

For a gene Gene1, the mean of the dCt values of all reference samples is calculated (if applicable).

The resulting mean dCt value of all reference samples is subtracted from the corresponding dCt value of Gene1 in sample A. The resulting value is called ddCt.

The transformation x→2^{-x}, is applied to each ddCt value. The resulting value is called exprs. This value is equivalent to a relative expression level or an X-fold change.

For each value an error is calculated, which is based on the standard error of the mean.

This analysis was carried out for each patient separately. The first sample (cycle 1, day 1, hour 0) was used as the reference sample. The ‘exprs’ value indicates relative expression levels (fold change values) for individual samples.

### Median/Average Gene Expression Values

Depending on the data distribution, it might be preferable to use either the median or the average value. In the case where the distribution of expression levels over the patients is non-normal, as in the present examples, the median value is usually preferred over the average value, therefore accounting for a possible skewed data distribution. In order to prove repetitive behavior of the gene expression, an example is given to show the expression level over the treatment period. [FIG. 4]

Table 5 contains the median expression level values for treatment day 5 (cycle 1, day 5, hours 0, 2, and 5) at a daily dose of 600 mg or 800 mg Resminostat mesylate salt is given below.

TABLE 5
Median expression level at cycle 1, day 5. First 3 genes
(18S, GAPDH, TBP) represent housekeeping genes

| | | Median | Median |
|---|---|---|---|
| Marker | Hour | 600 mg dose | 800 mg dose |
| 18S | 0 | 0.872 | 0.635 |
| 18S | 2 | 0.871 | 0.682 |
| 18S | 5 | 0.778 | 0.550 |
| GAPDH | 0 | 1.092 | 1.136 |
| GAPDH | 2 | 1.201 | 1.362 |
| GAPDH | 5 | 1.296 | 1.228 |
| TBP | 0 | 0.991 | 1.305 |
| TBP | 2 | 0.994 | 1.071 |
| TBP | 5 | 1.052 | 1.286 |
| CCDC43 | 0 | 0.917 | 1.391 |
| CCDC43 | 2 | 0.318 | 0.329 |
| CCDC43 | 5 | 0.378 | 0.319 |
| DPP3 | 0 | 1.121 | 1.457 |
| DPP3 | 2 | 0.767 | 0.912 |
| DPP3 | 5 | 0.267 | 0.395 |
| HIST2H4A; HIST2H4B | 0 | 0.875 | 1.530 |
| HIST2H4A; HIST2H4B | 2 | 2.111 | 3.345 |
| HIST2H4A; HIST2H4B | 5 | 2.042 | 2.821 |
| KDELC2 | 0 | 0.909 | 1.141 |
| KDELC2 | 2 | 0.544 | 0.509 |
| KDELC2 | 5 | 0.215 | 0.215 |
| MICALL1 | 0 | 0.981 | 1.024 |
| MICALL1 | 2 | 0.395 | 0.407 |
| MICALL1 | 5 | 0.201 | 0.163 |
| ZFP64 | 0 | 0.980 | 1.280 |
| ZFP64 | 2 | 0.412 | 0.356 |
| ZFP64 | 5 | 0.228 | 0.188 |

### Example 3a

### ZFP64 Gene Expression Correlated with SD/PD

### ZFP64 as Prognostic Marker

Statistical analyses were carried out using the statistical software R (R_Core_Team, 2012. R: A language and environment for statistical computing. [Online] Available at: http://www.R-project.org).

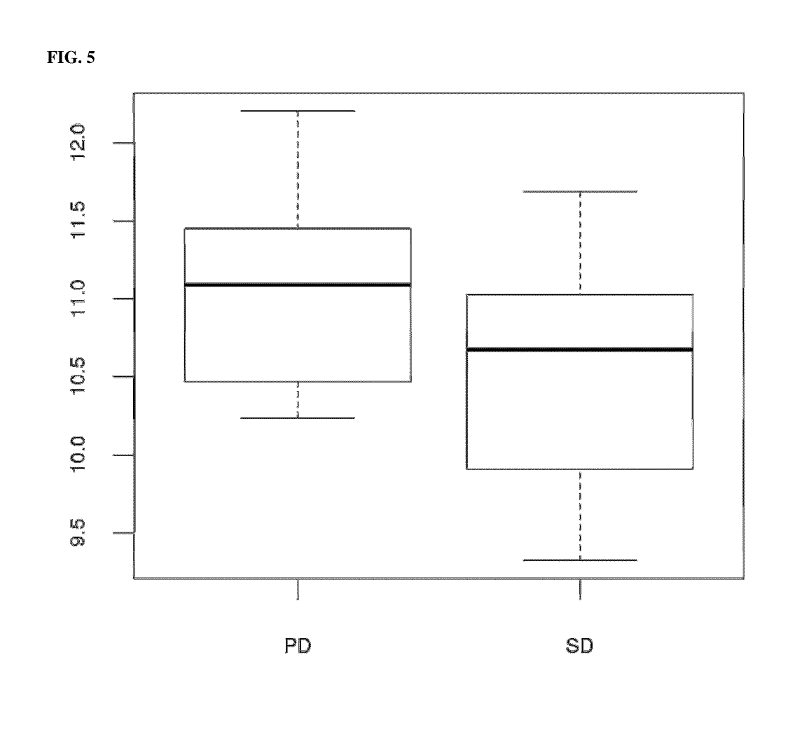

Statistical analysis of the baseline expression of the gene ZFP64 (Cycle 1, Day 1, Hour 0) revealed that, based on ZFP64 baseline gene expression, the patients can be separated into two groups, namely into a) patients which are expected to show a positive outcome of an HDAC inhibitor treatment as described herein, and b) patients which are expected not to show a positive outcome of an HDAC inhibitor treatment as described herein. The difference at baseline for ZFP64 gene expression is detectable by using the dCt values, not the expression level (for mathematical connection between the two, refer to ‘Basic concept of the ΔΔCt method’, see above). FIG. 5 shows a box plot of the two patient groups from the SAPHIRE clinical study with their respective median values, interquartile range (25^{th} to 75^{th} percentile) and data range. According to a Welch two sample t-test, the p-value (two-sided) for the difference between the two groups is 0.03.

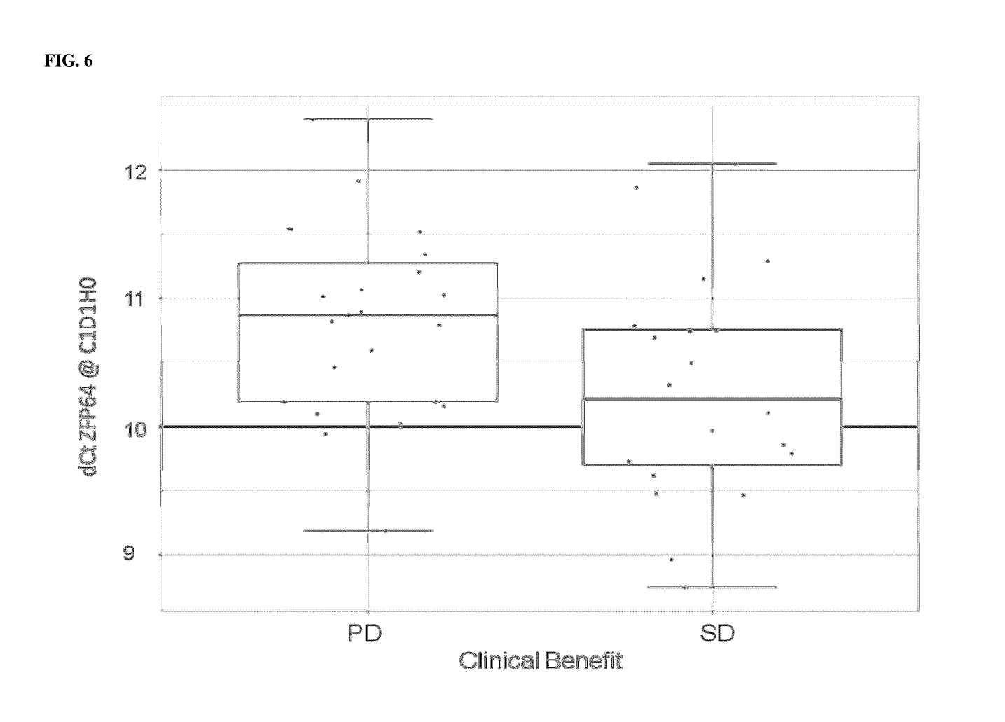

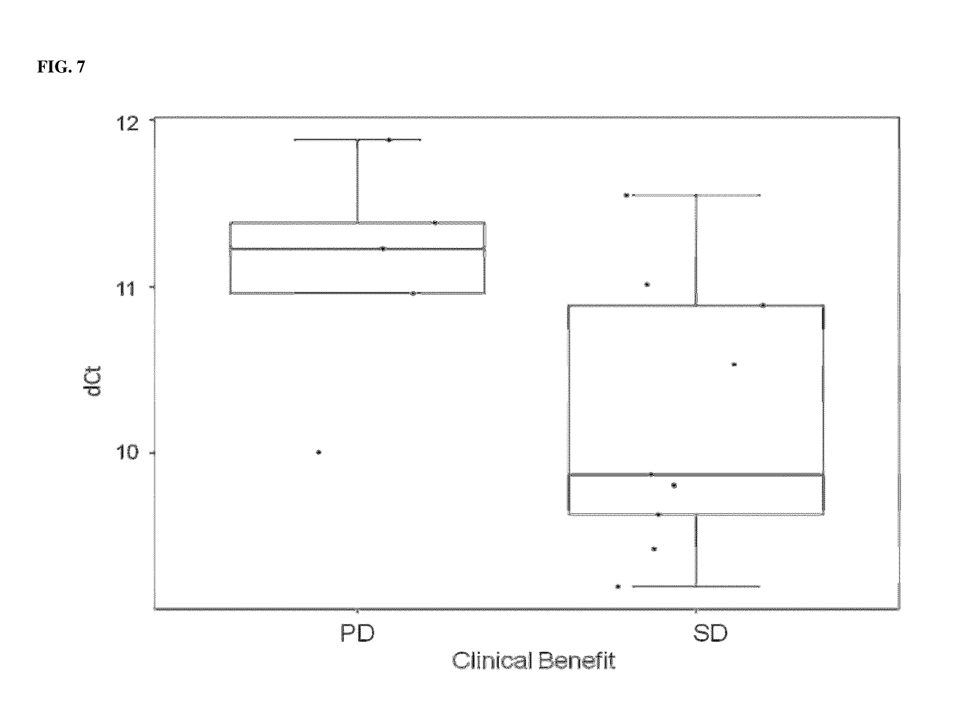

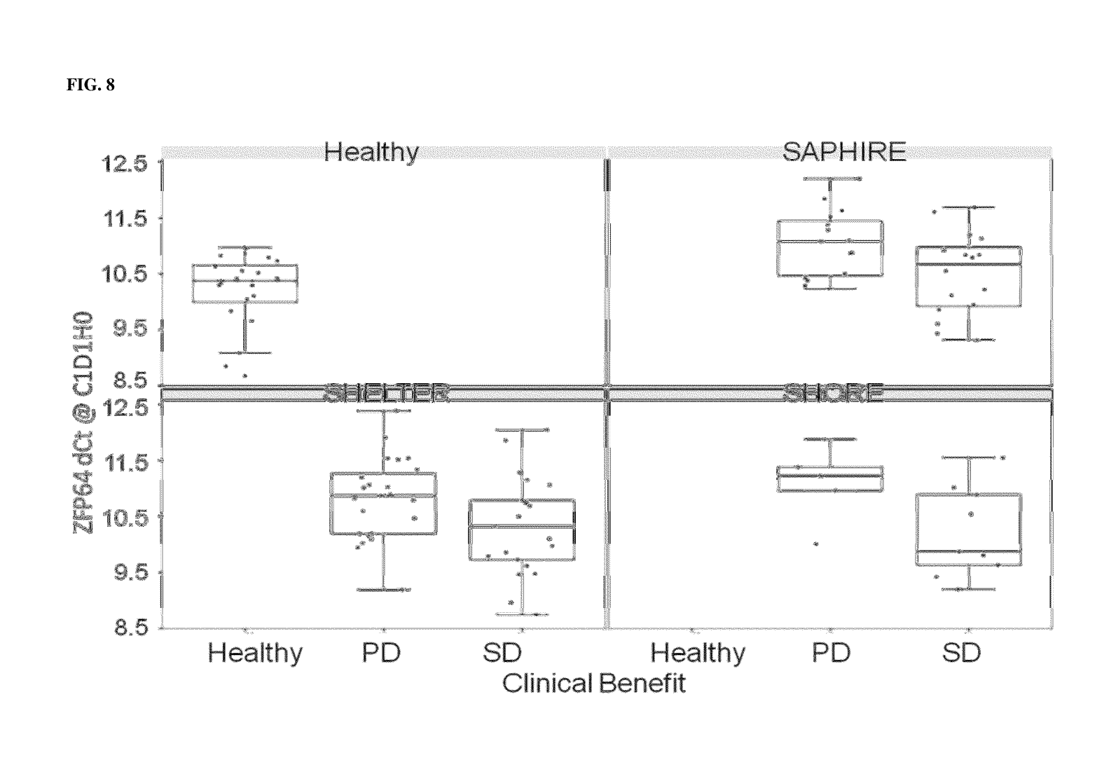

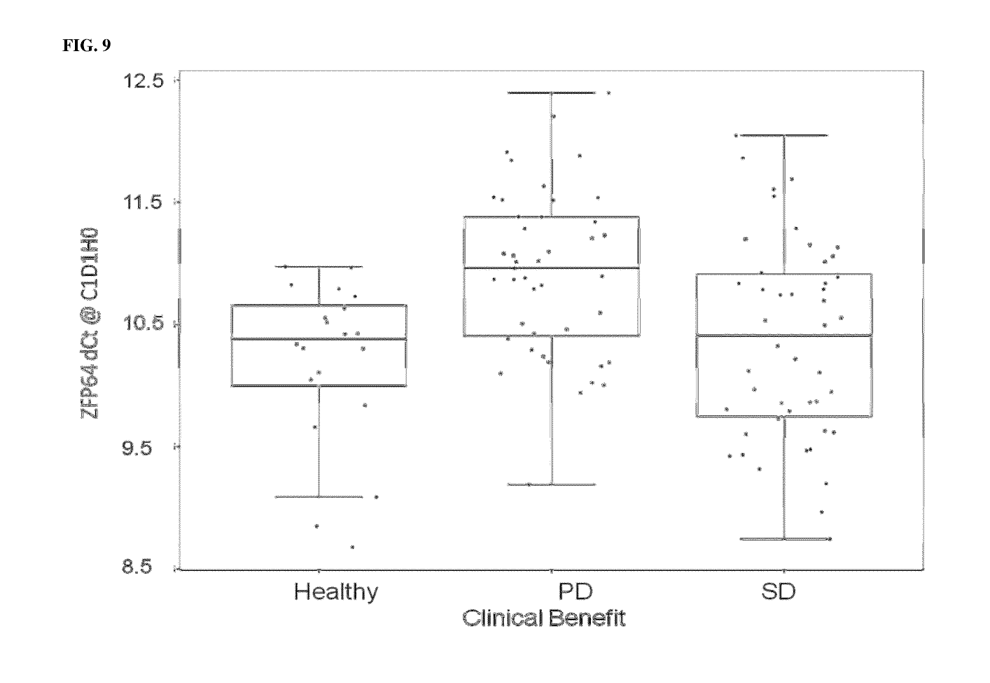

Accordingly, in FIGS. 6 and 7, the respective plots are shown for the SHELTER (p-value=0.04) and SHORE (p-value=0.06) clinical studies, indicating the applicability of ZFP64 as marker under resminostat treatment. Additionally, blood samples from healthy donors were taken and mRNA expression was measured as described above for the clinical studies. dCt values from healthy donors were in the expression range of the clinical samples with the respective median of the patients with stable disease (SD) in closer proximity of the median of the healthy donors. This is shown as boxplot diagram in FIG. 8 for the individual studies and in FIG. 9 as a combination of all studies.

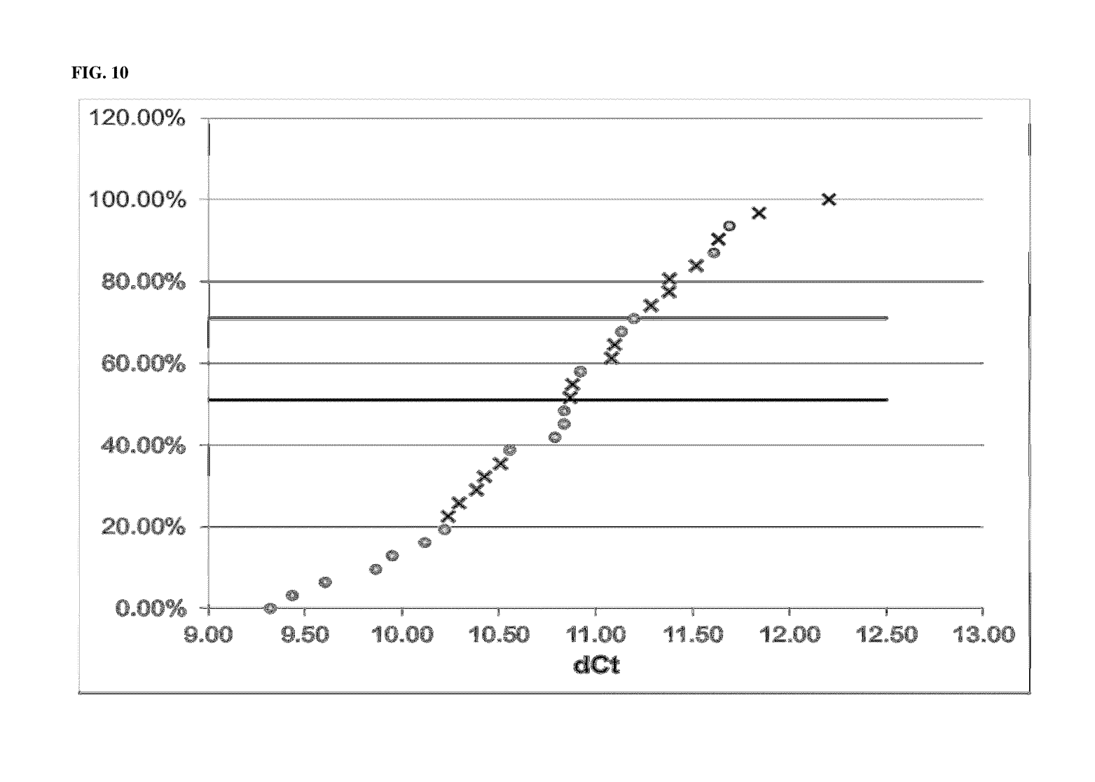

Considering the prognostic value of ZFP64 as biomarker, a separation into three groups of prognostic power is possible based on the percentile ranking of the dCt values (see FIG. 10). A dCt value in the range including the 71^{st} percentile and above indicates with a precision of 0.78 (7 of 9) that the HDAC inhibitor treatment does not result in a positive outcome. A dCt value in the range including the 51^{st} percentile and below, indicates a positive outcome of the HDAC inhibitor treatment with a 0.69 (11 of 16) precision. A dCt value in the range between, but not including the 51^{st} and 71^{st} percentile does not give a definitive prognostic indication. The percentile ranges of dCt values as described above relate to the overall distribution of gene expression over all patients receiving the HDAC inhibitor treatment.

### Example 3b

### ZFP64 Gene Expression Correlated with OS/PFS

### Splitting Methodology

Based on the results and indications seen in the analysis in example 3a, the dCt values of ZFP64 at baseline were treated with the method described here for determining the split between gene expression groups. Data was processed as follows: Gene expression data of the genes of interest was gathered as real time PCR dCt values relative to the expression of housekeeping genes for a specific time point, e.g. at baseline prior to treatment start. The patient cohort was split stepwise at defined percentiles (in steps of 5 percentiles, totaling from the 25^{th} percentile through 85^{th} percentile) of dCt values for the gene of interest into two groups: low and high values, relative to each other. Said two groups were then compared in Kaplan-Meier analyses for OS, PFS in order to identify a statistically significant difference that separates the patients' probability for OS, PFS.

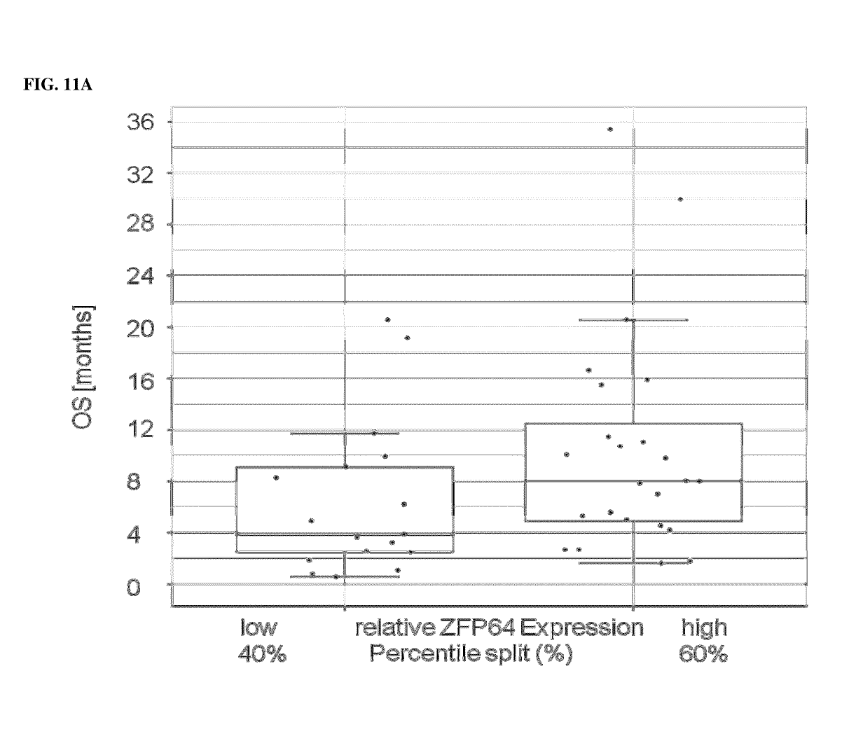

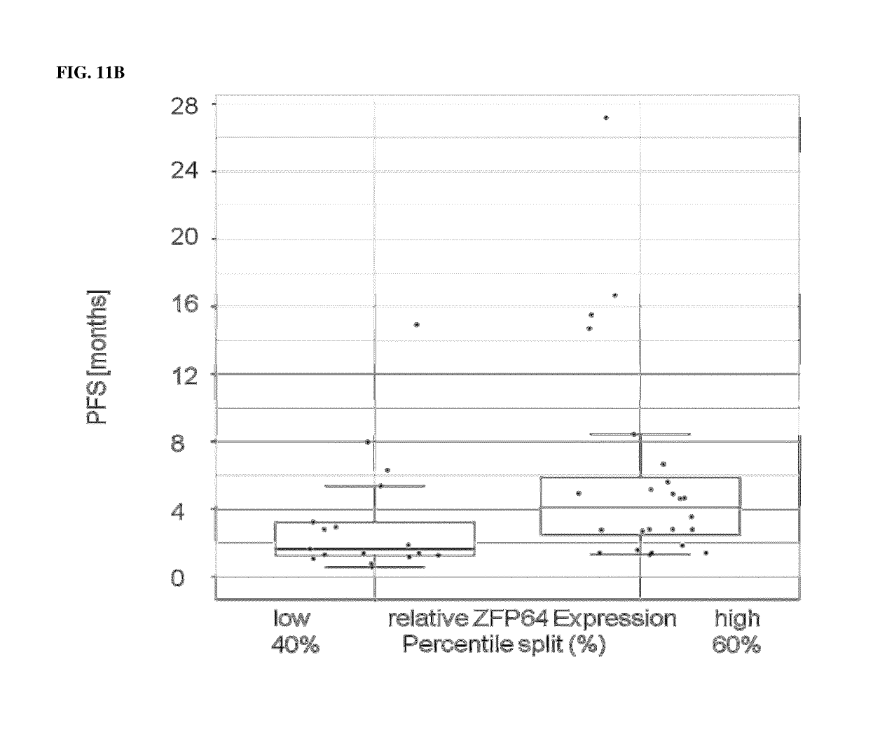

Based on data and percentile ranking in FIG. 10 and the splitting methodology as described above, a low and a high group are defined as shown in and detailed for FIGS. 11 and 11b.

Said splits are applied to boxplot diagrams, displaying the overall survival (OS) in FIG. 11 and progression-free survival (PFS) in FIG. 11b exemplified for the data from the SHELTER study. This analysis shows a statistical difference between the high and low group, with a p-value of 0.06 for the correlation with OS and a p-value Of 0.03 for the correlation with PFS.

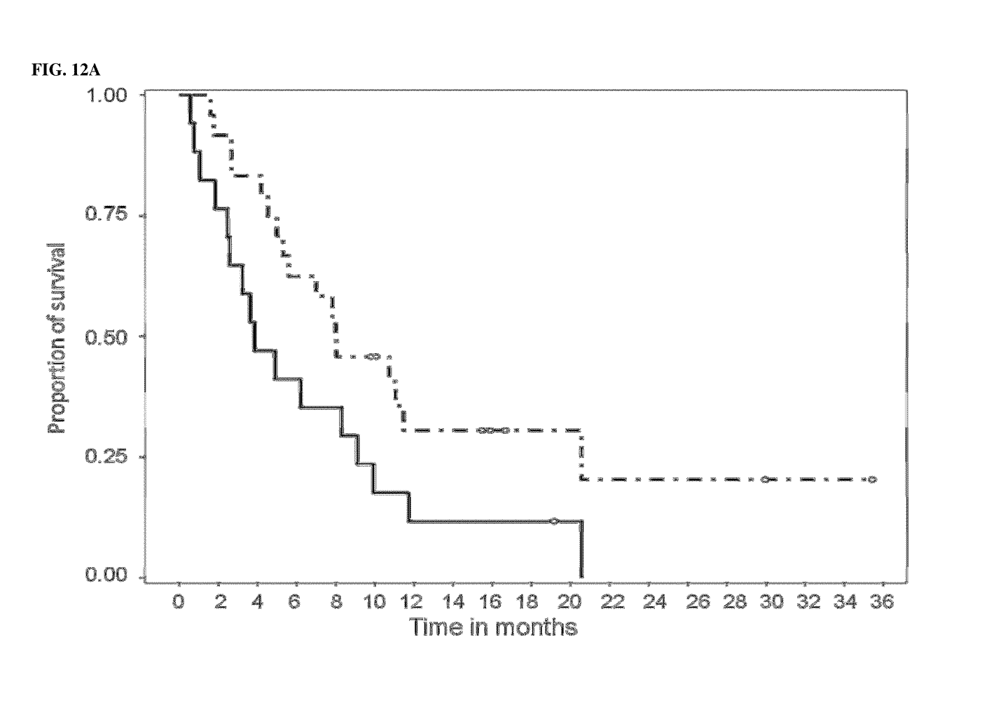

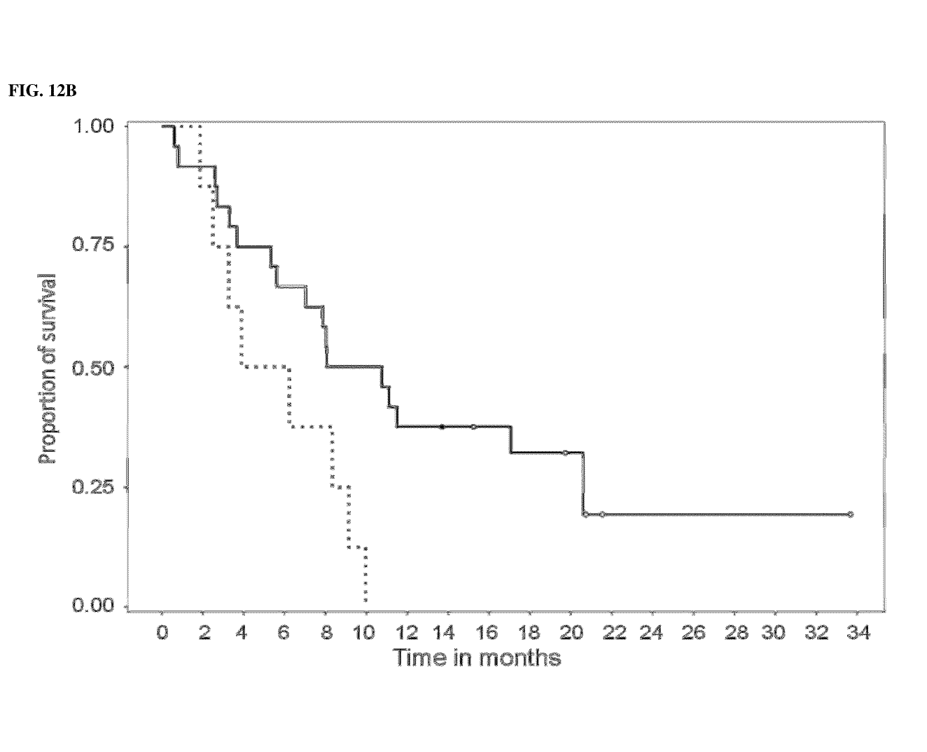

A Kaplan-Meier analysis of said data, as seen in FIGS. 12 and 12b, reflects the results from FIGS. 11 and 11b. In FIG. 12, all available patients from the SHELTER trial are included in the analysis with respect to OS. The split is comparable to the one in FIG. 11, with 60% of the patients in the high expression group and 40% in the low expression group, with a statistically determined p-value of 0.04, using the log-rank test. The median OS (high ZFP64 expression) is 8 months (95% C.I.: 5.6-NA), whereas the median OS (low ZFP64 expression) is 3.9 months (95% C.I.: 2.6-9.9).

In FIG. 12b, only those patients from the SHELTER study are included in the analysis, who were either treated with resminostat (600 mg) or a combination of resminostat (600 mg) and sorafenib (400 mg). The percentile for the split is different than in FIG. 12, as are the median values for the respective groups. The median OS (high ZFP64 expression is: 9.4 months (95% C.I.: 7.0-20.6) and the median OS (low ZFP64 expression) is 5.1 months (95% C.I.: 3.3-NA) with a p-value determined by log-rank test of 0.02.

Together with FIG. 10, FIGS. 12 and 12b indicate the same trend. The center part in FIG. 10 between 51% and 71% is within the same range as the two percentiles used for splitting in FIGS. 12 and 12b (60^{th} percentile and 75^{th} percentile, respectively). Keep in mind that a higher dCt ZFP64 baseline value means a lower ZFP64 mRNA expression and is indicative of developing progressive disease (PD) in HCC patients upon treatment with resminostat, whereas a lower dCt ZFP64 baseline value (i.e. higher ZFP64 mRNA expression) is indicative of developing stable disease (SD).

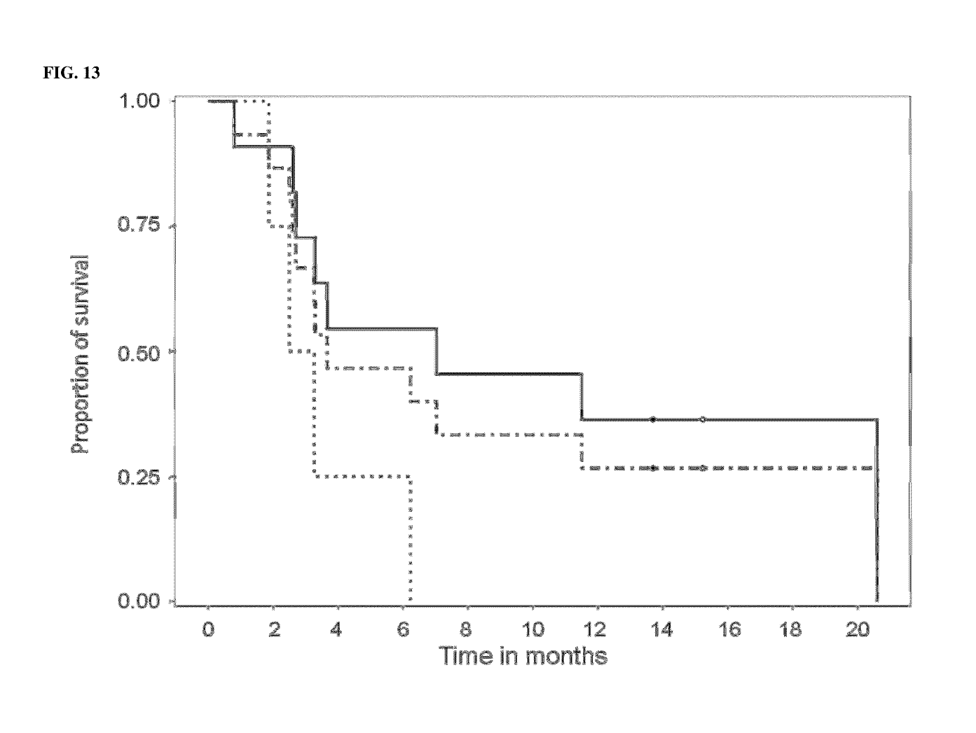

In FIG. 13, the baseline expression of evaluable patients (Some patients participating in the clinical trial could not be included into this analysis due to missing data or low quality samples) from the SHELTER study receiving resminostat (600 mg) is analyzed. The split shown is at the 75th percentile, resulting in a median OS for low ZFP64 expression of 0.9 months (95% C.I.: 1.9-NA) and a median OS for high ZFP64 expression of 7.0 months (95% C.I.: 3.3-NA) with a log-rank determined p-value of 0.05. The overall median OS for the two groups is 3.7 months, represented by the intermittent line.

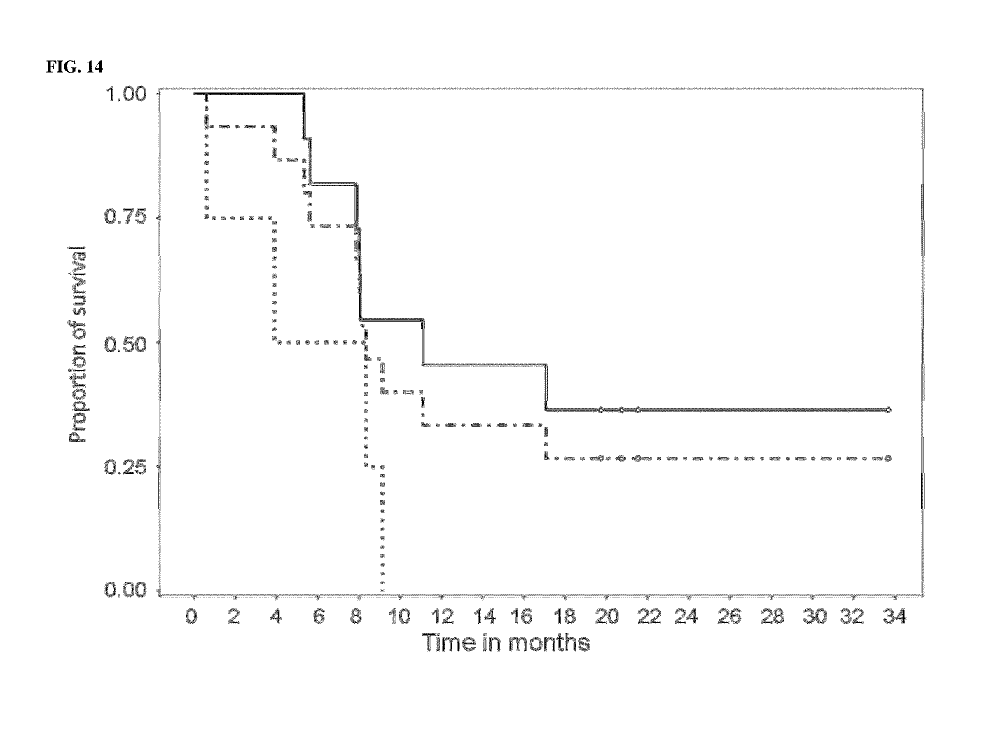

In FIG. 14, the baseline expression of evaluable patients (see above) from the SHELTER study receiving the combination resminostat (600 mg) and sorafenib (400 mg) is analyzed. The split shown is at the 75th percentile, resulting in a median OS for low ZFP64 expression of 6.1 months (95% C.I.: 0.6-NA) and a median OS for high ZFP64 expression of 11.1 months (95% C.I.: 8.0-NA) with a log-rank determined p-value of 0.07. The overall median OS for the two groups is 8.3 months, represented by the intermittent line.

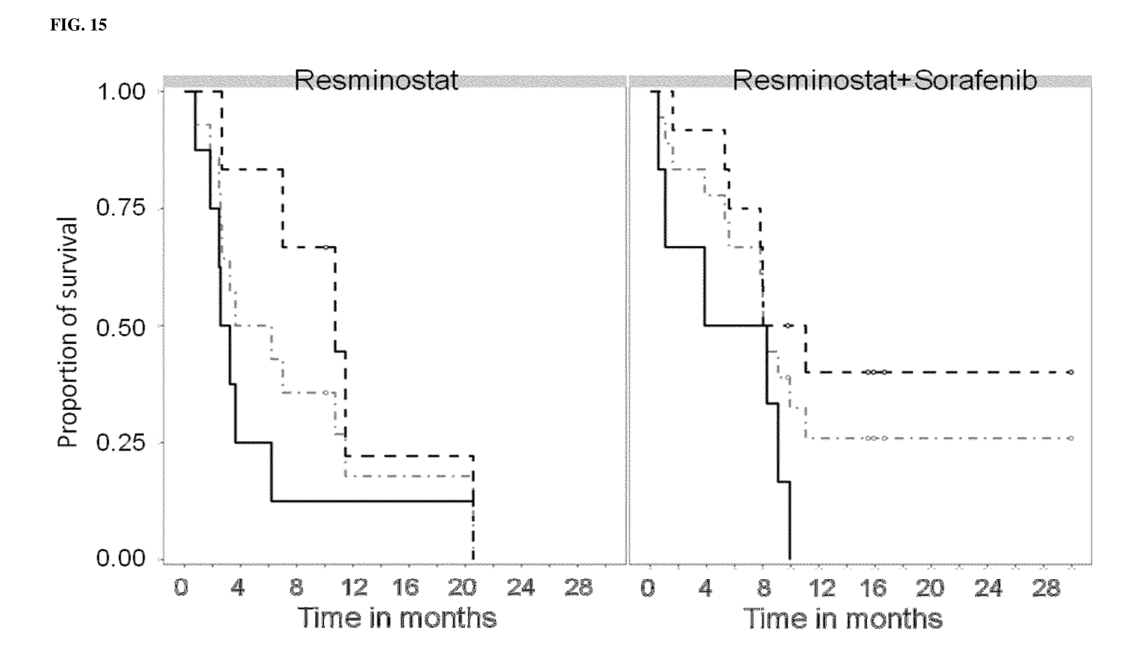

FIG. 15 shows the evaluable patients (see above) from the SHELTER study receiving resminostat or the combination of resminostat and sorafenib side by side, respectively. The split is calculated by the method described herein, and based on the overall study population (as seen in FIG. 11). The two graphs essentially mirror the trend seen in FIGS. 13 and 14, respectively, displaying only differences due to the fact that the values used for splitting into two groups differ. Since the percentiles (75^{th} for FIGS. 13 and 14 and 60^{th} for FIG. 15) are comparable to those seen in FIG. 10, where areas of definitive outcome predictability, and areas wherein a solid predictability is not given, are defined, the applicability of ZFP64 as marker is confirmed.

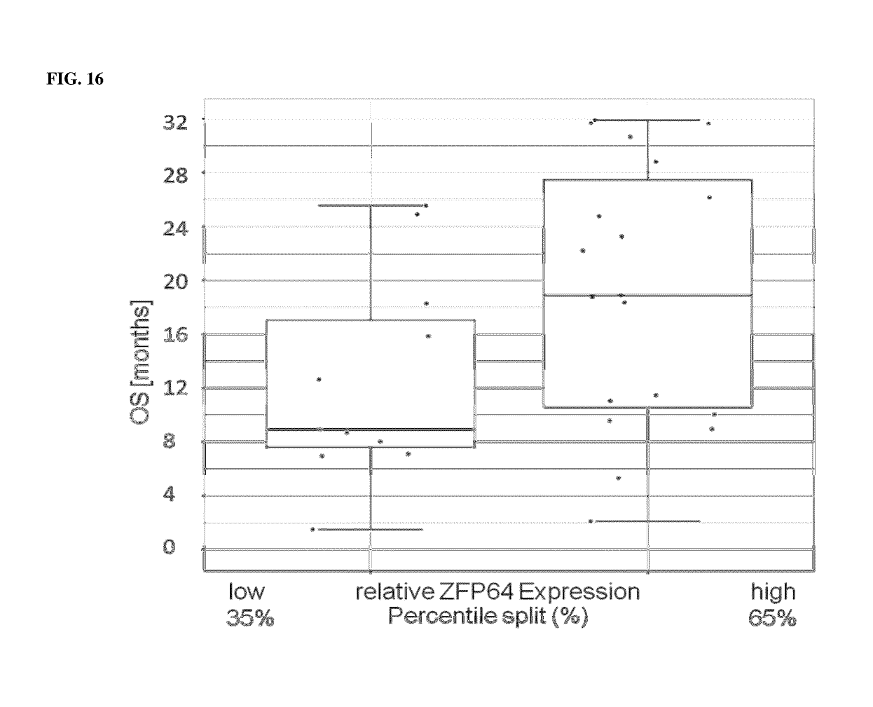

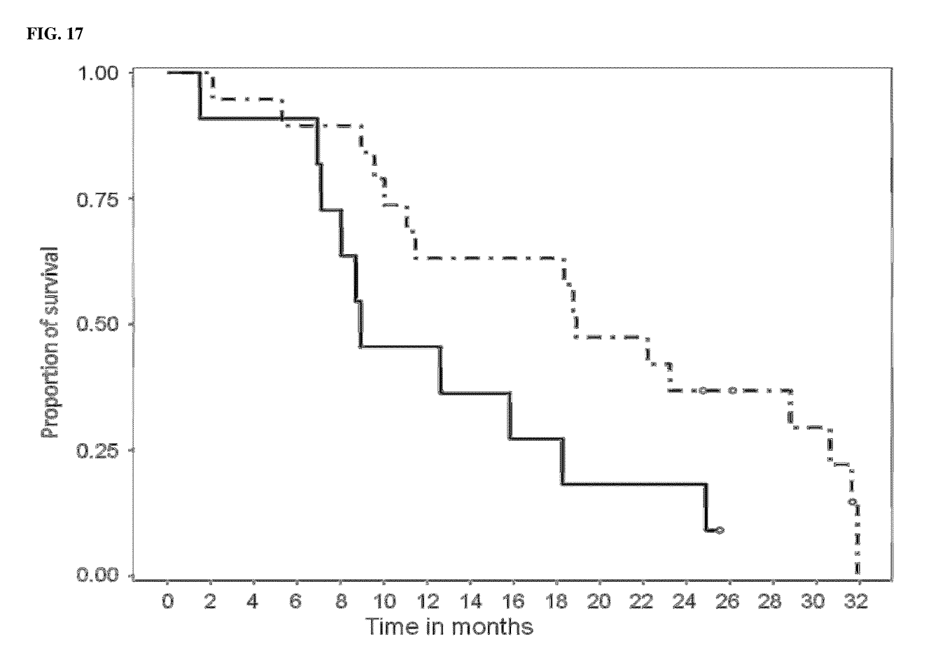

FIGS. 16 and 17 represent the analysis for the SAPHIRE data, based on the same methods as described in this section for the SHELTER data analysis. The boxplot diagram in FIG. 16 displays the overall survival (OS) data with respect to the split at the 65^{th} percentile into high and low ZFP64 expression, showing a statistical difference between the two groups, with a p-value by log-rank test of 0.04. FIG. 17 shows the Kaplan-Meier analysis of OS with the split of ZFP64 expression at the 65^{th} percentile. The p-value by log-rank test is 0.04.

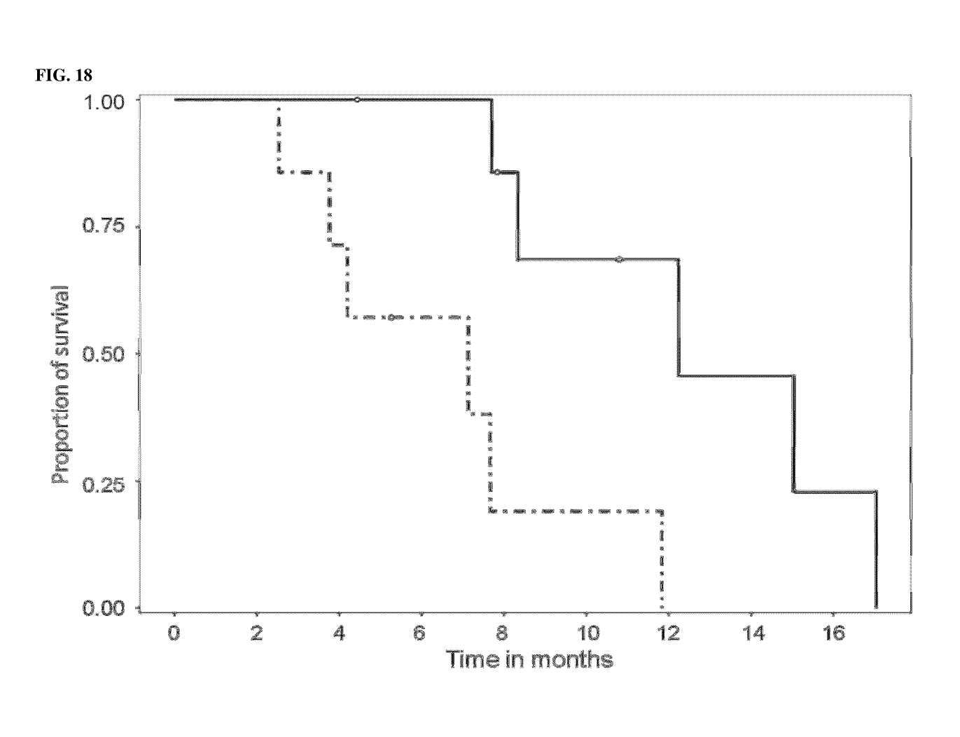

FIG. 18 is the respective Kaplan-Meier analysis for the SHORE study data. The data were collected before the final study report and some censored patients (circles) are above the 0.5 proportion of the survival lines, therefore the median value for OS in the respective groups could differ to some degree upon final evaluation of all patient data. Nevertheless, a similar trend is seen in CRC as in HCC and HL, with the relative low ZFP64 expression group showing a shorter OS and the relative high ZFP64 expression group showing a longer OS.

### DPP3 as Prognostic Marker

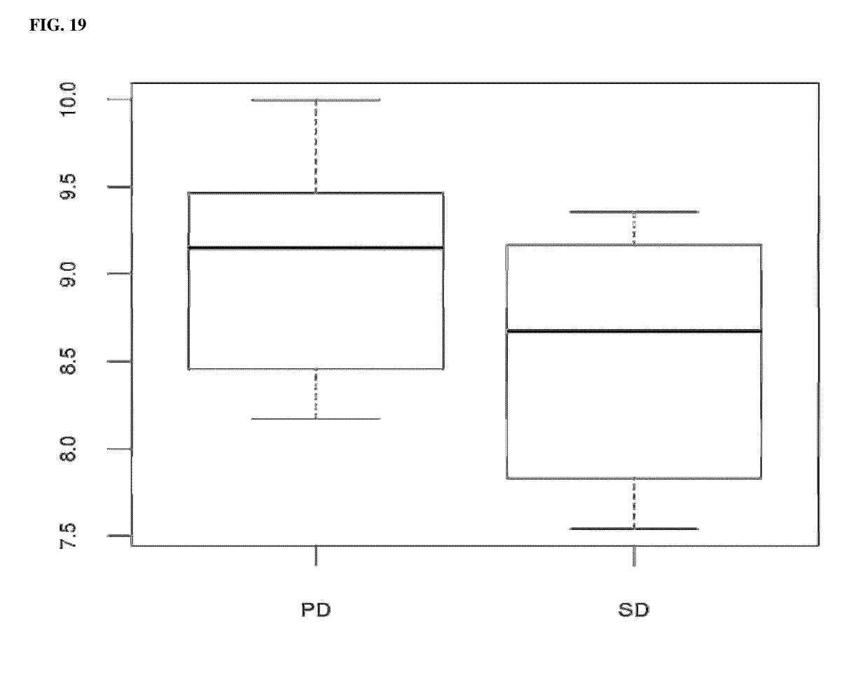

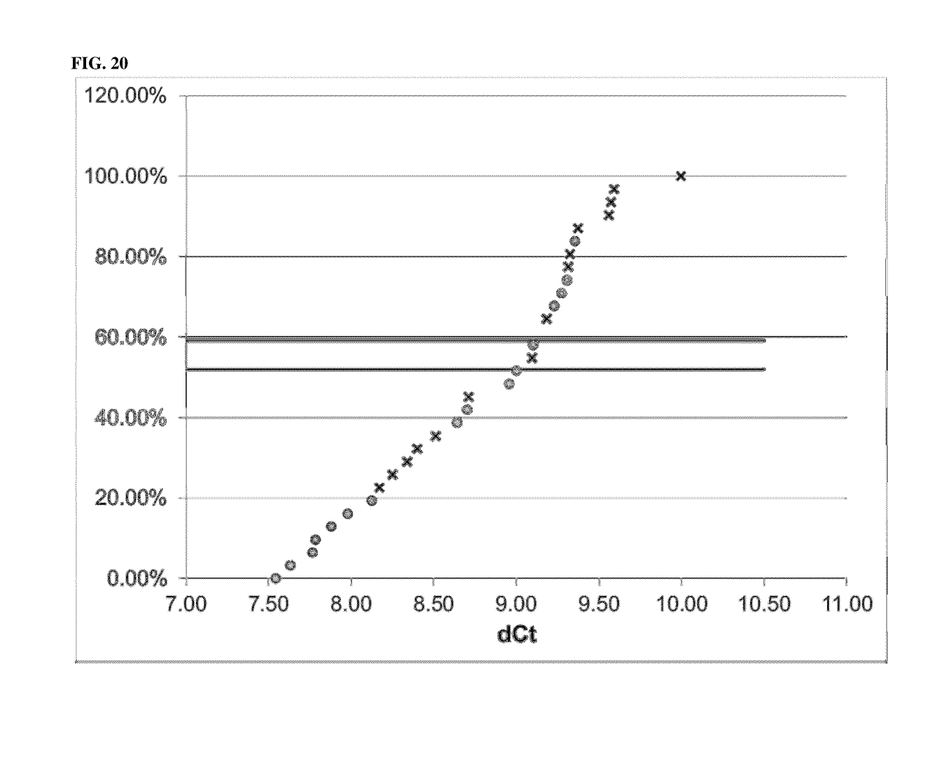

Statistical analysis of the baseline expression of the gene DPP3 (Cycle 1, Day 1, Hour 0) revealed that, based on DPP3 baseline gene expression, the patients can be separated into two groups, namely into a) patients which are expected to show a positive outcome of an HDAC inhibitor treatment as described herein, and b) patients which are expected not to show a positive outcome of an HDAC inhibitor treatment as described herein. The difference at DPP3 baseline gene expression is detectable by using the dCt values, not the expression level FIG. 19 shows a box plot of the two patient groups with their respective median values, interquartile range (25^{th} to 75^{th} percentile) and data range. The two sided p-value for the difference is 0.03, according to a Welch two sample t-test. Considering the prognostic value of DPP3 as biomarker, a separation into three groups of prognostic power is done based on the percentile ranking of the dCt values (see FIG. 20).

A dCt value in the range including the 59^{th} percentile and above indicates with a precision of 0.69 (9 of 13) that the HDAC inhibitor treatment does not result in a positive outcome. A dCt value in the range including the 52^{nd} percentile and below, indicates a positive outcome of the HDAC inhibitor treatment with a 0.64 (11 of 17) precision. A dCt value in the range between, but not including the 52^{nd} and 59^{th} percentile does not give a definitive prognostic indication. The percentile ranges of dCt values as described above relate to the overall distribution of gene expression over all patients receiving the HDAC inhibitor treatment.

It has been shown that resminostat administration leads to down-regulation of ZFP64 gene expression in cancer cell lines, healthy donor PBMCs and whole blood cells as well as in whole blood cells taken from patients in clinical trials SHELTER, SAPHIRE, and SHORE.

The relative gene expression in clinical trials at baseline (cycle1, day1, hour0) is indicative of the clinical outcome for the patients under resminostat treatment, namely the evaluation of progressive disease (PD) or at least stable disease or even responsive disease (SD). Higher ZFP64 expression levels measured at baseline (prior to treatment start) in cancer patients are indicative of larger clinical benefit (PD vs SD, increase of PFS and OS times) upon treatment with resminostat. Additionally and more relevant, said relative gene expression at baseline is also indicative for progression-free survival (PFS) and/or overall survival (OS) time of patients under resminostat treatment, showing a statistically relevant difference between defined relative high and relative low expression groups.

Cell and whole blood experiments prove that the gene regulative effect of resminostat is not influenced by sorafenib. A combination of resminostat and sorafenib does show comparable values of down-regulation of ZFP64 gene expression, compared with the down-regulation for resminostat alone.

ZFP64 is a pharmacodynamic marker for resminostat activity.

ZFP64 indicated as prognostic as well as predictive biomarker for resminostat response. Furthermore, ZFP64 offers the opportunity for the development of a companion diagnostic for patient stratification.

### Raw Data for Figures (Table Number is Equivalent to the Respective Figure Number):

TABLE F2/F3
Comparison between change in gene expression upon HDAC
inhibitor administration, as determined in samples of peripheral
blood (ex vivo) and selected human cancer cell lines (in vitro).

| Gene | Time point | Blood | HepG2 | HT29 |
|---|---|---|---|---|
| CCDC43 | 0 h | 1.00 | 1.00 | 1.00 |
| | 2 h | 0.29 | 0.70 | 0.82 |
| | 5 h | 0.35 | 0.46 | 0.53 |
| DPP3 | 0 h | 1.00 | 1.00 | 1.00 |
| | 2 h | 0.57 | 0.86 | 0.87 |
| | 5 h | 0.29 | 0.72 | 0.72 |
| HIST2H4A/B | 0 h | 1.00 | 1.00 | 1.00 |
| | 2 h | 2.59 | 0.88 | 0.96 |
| | 5 h | 4.11 | 1.17 | 0.88 |
| KDELC2 | 0 h | 1.00 | 1.00 | 1.00 |
| | 2 h | 0.47 | 0.84 | 0.79 |
| | 5 h | 0.07 | 0.54 | 0.35 |
| MICALL1 | 0 h | 1.00 | 1.00 | 1.00 |
| | 2 h | 0.30 | 0.85 | 0.81 |
| | 5 h | 0.10 | 0.50 | 0.42 |
| ZFP64 | 0 h | 1.00 | 1.00 | 1.00 |
| | 2 h | 0.24 | 0.46 | 0.58 |
| | 5 h | 0.15 | 0.19 | 0.25 |
| 18s | 0 h | 1.00 | 1.00 | 1.00 |
| | 2 h | 0.83 | 0.91 | 0.94 |
| | 5 h | 0.66 | 0.87 | 0.91 |
| GAPDH | 0 h | 1.00 | 1.00 | 1.00 |
| | 2 h | 1.16 | 0.96 | 0.93 |
| | 5 h | 1.38 | 1.03 | 0.97 |
| TBP | 0 h | 1.00 | 1.00 | 1.00 |
| | 2 h | 1.03 | 1.15 | 1.15 |
| | 5 h | 1.11 | 1.12 | 1.13 |

TABLE F6
dCt (ZFP64)

| PD | SD |
|---|---|
| 9.19 | 8.75 |
| 9.94 | 8.97 |
| 10.02 | 9.47 |
| 10.10 | 9.48 |
| 10.16 | 9.62 |
| 10.19 | 9.73 |
| 10.19 | 9.79 |
| 10.46 | 9.86 |
| 10.60 | 9.97 |
| 10.79 | 10.11 |
| 10.82 | 10.33 |
| 10.87 | 10.50 |
| 10.89 | 10.69 |
| 11.01 | 10.74 |
| 11.02 | 10.75 |
| 11.06 | 10.79 |
| 11.20 | 11.15 |
| 11.34 | 11.29 |
| 11.52 | 11.86 |
| 11.54 | 12.05 |
| 11.54 | |
| 11.91 | |
| 12.39 | |

TABLE F5
dCt (ZFP64)

| PD | SD |
|---|---|
| 10.24 | 9.32 |
| 10.29 | 9.43 |
| 10.38 | 9.60 |
| 10.43 | 9.86 |
| 10.51 | 9.95 |
| 10.87 | 10.12 |
| 10.88 | 10.22 |
| 11.08 | 10.56 |
| 11.10 | 10.79 |
| 11.29 | 10.84 |
| 11.38 | 10.84 |
| 11.52 | 10.92 |
| 11.63 | 11.13 |
| 11.84 | 11.20 |
| 12.20 | 11.61 |
| | 11.69 |

TABLE F7

| dCt (ZFP64) | Disease state | dCt (ZFP64) | Disease state |
|---|---|---|---|
| 10.005 | PD | 9.198 | SD |
| 10.96 | PD | 9.424 | SD |
| 11.228 | PD | 9.631 | SD |
| 11.383 | PD | 9.806 | SD |
| 11.882 | PD | 9.872 | SD |
| | | 10.534 | SD |
| | | 10.887 | SD |
| | | 11.014 | SD |
| | | 11.548 | SD |

TABLE F8

| | SAPHIRE | | SHELTER | | SHORE | |
|---|---|---|---|---|---|---|
| Healthy | PD | SD | PD | SD | PD | SD |
| 8.68 | 10.238259 | 9.3202401 | 9.19 | 8.747 | 10.005 | 9.198 |
| 8.851 | 10.293933 | 9.4346557 | 9.9433254 | 8.965 | 10.96 | 9.424 |
| 9.089 | 10.384568 | 9.6037827 | 10.022981 | 9.4680678 | 11.228 | 9.631 |
| 9.661 | 10.426038 | 9.8647959 | 10.099 | 9.477 | 11.383 | 9.806 |
| 9.838 | 10.508729 | 9.9492891 | 10.16 | 9.618 | 11.882 | 9.872 |
| 10.048 | 10.867462 | 10.117474 | 10.192 | 9.729 | | 10.534 |
| 10.105 | 10.880343 | 10.219671 | 10.192 | 9.791 | | 10.887 |
| 10.303 | 11.081816 | 10.556121 | 10.463 | 9.8589718 | | 11.014 |
| 10.305 | 11.097414 | 10.789325 | 10.596095 | 9.970357 | | 11.548 |
| 10.339 | 11.285386 | 10.836466 | 10.791 | 10.106 | | |
| 10.423 | 11.380366 | 10.83761 | 10.821 | 10.325 | | |
| 10.426 | 11.518661 | 10.921212 | 10.87 | 10.496 | | |
| 10.52 | 11.633498 | 11.132758 | 10.894 | 10.692 | | |
| 10.555 | 11.842571 | 11.197216 | 11.013 | 10.743 | | |
| 10.631 | 12.203387 | 11.609842 | 11.023 | 10.748 | | |
| 10.731 | | 11.689532 | 11.064716 | 10.786308 | | |
| 10.792 | | | 11.204 | 11.06 | | |
| 10.825 | | | 11.341 | 11.152 | | |
| 10.964 | | | 11.516 | 11.288 | | |
| 10.973 | | | 11.536578 | 11.861 | | |
| | | | 11.54 | 12.047 | | |
| | | | 11.911 | | | |
| | | | 12.393 | | | |

TABLE F11
OS (months)

| low (40%) | high (60%) |
|---|---|
| relative | relative |
| expression | expression |
| 0.6 | 1.6 |
| 0.8 | 1.8 |
| 1.1 | 2.7 |
| 1.8 | 2.7 |
| 2.5 | 4.2 |
| 2.6 | 4.5 |
| 3.2 | 5 |
| 3.6 | 5.3 |
| 3.9 | 5.6 |
| 4.9 | 7 |
| 6.2 | 7.8 |
| 8.3 | 8 |
| 9.1 | 8 |
| 9.9 | 9.8 |
| 11.7 | 10.1 |
| 19.2 | 10.7 |
| 20.6 | 11.1 |
| | 11.5 |
| | 15.5 |
| | 15.9 |
| | 16.7 |
| | 20.6 |
| | 30 |
| | 35.4 |

TABLE F11b
PFS (months)

| low (40%) | high (60%) |
|---|---|
| relative | relative |
| expression | expression |
| 0.6 | 1.3 |
| 0.8 | 1.4 |
| 1.1 | 1.4 |
| 1.2 | 1.4 |
| 1.3 | 1.6 |
| 1.3 | 1.8 |
| 1.4 | 2.7 |
| 1.4 | 2.8 |
| 1.6 | 2.8 |
| 1.9 | 2.8 |
| 2.8 | 2.8 |
| 2.9 | 3.5 |
| 3.2 | 4.6 |
| 5.4 | 4.7 |
| 6.3 | 4.9 |
| 8 | 4.9 |
| 14.9 | 5.2 |
| | 5.6 |
| | 6.7 |
| | 8.4 |

TABLE F16
OS (months)

| low (35%) | high (65%) |
|---|---|
| relative | relative |
| expression | expression |
| 1.5 | 2.1 |
| 6.9 | 5.3 |
| 7.1 | 9 |
| 8 | 9.6 |
| 8.7 | 10 |
| 8.9 | 11.1 |
| 12.6 | 11.5 |
| 15.8 | 18.3 |
| 18.3 | 18.8 |
| 24.9 | 18.9 |
| 25.5 | 22.2 |
| | 23.2 |
| | 24.8 |
| | 26.1 |
| | 28.8 |
| | 30.6 |
| | 31.6 |
| | 31.7 |
| | 31.9 |

1. A method
(I) of determining an effect of an HDAC inhibitor treatment, the method comprising the following steps:
a) Providing a sample of a patient receiving said HDAC inhibitor treatment,
b) determining the gene expression and/or the change of the gene expression of at least one gene selected from the group comprising ZFP64, DPP3, CCDC43, HIST2H4A/B, KDELC2 and MICALL1 in said sample,
c) correlating the determined gene expression and/or the change of the gene expression of said at least one gene to an effect of said HDAC inhibitor treatment in said patient;
or
(II) of monitoring an HDAC inhibitor treatment, the method comprising the following steps:
a) Providing a sample of a patient receiving said HDAC inhibitor treatment,
b) determining the gene expression and/or the change of the gene expression of at least one gene selected from the group comprising ZFP64, DPP3, CCDC43, HIST2H4A/B, KDELC2 and MICALL1 in said sample,
c) repeating the above steps a and b at least once, preferably more than once, and
d) using said gene expressions determined in steps a) to c) to generate a time profile of said patient's response to said HDAC inhibitor treatment;
or
(III)
of stratification of a patient potentially in need of an HDAC inhibitor treatment comprising the following steps:
a) Providing a sample of said patient
b) Determining the gene expression of at least one gene selected from the group comprising ZFP64, DPP3, CCDC43, HIST2H4A/B, KDELC2 and MICALL1 in said sample
c) Correlating the determined gene expression of said at least one gene to the probability that an HDAC inhibitor treatment has a beneficial effect on said patient and
d) classifying said patient as responder or non-responder to said HDAC inhibitor treatment, based on the probability determined in step c;
or
(IV)
of predicting the probability of a positive outcome of an HDAC inhibitor treatment for a patient receiving said HDAC inhibitor treatment, the method comprising the following steps:
a) Providing a sample of said patient
b) Determining the gene expression of at least one gene selected from the group comprising ZFP64, DPP3, CCDC43, HIST2H4A/B, KDELC2 and MICALL1 in said sample,
c) Comparing said gene expression with the gene expression of said at least one gene in a sample provided from said patient prior to step a), and
d) Correlating the difference of the gene expression of said at least one gene in said sample provided in step a) and in said sample provided prior to step a) to the probability of a positive outcome of said HDAC inhibitor treatment for said patient;
or
(IV)
of determining the gene expression of at least one gene as pharmacodynamic marker in a patient in need of an HDAC inhibitor treatment, the method comprising the following steps:
a) Providing a sample of said patient,
b) determining the gene expression and/or the ZFP64, DPP3, CCDC43, HIST2H4A/B, KDELC2 and MICALL1 in said sample
c) correlating the determined gene expression and/or the change of the gene expression of said at least one gene to the relative inhibition of HDAC by the HDAC inhibitor.

2. A method of claim 1, which is method (II), and is for monitoring an HDAC inhibitor treatment, the method comprising the following steps:
a) Providing a sample of a patient receiving said HDAC inhibitor treatment,
b) determining the gene expression and/or the change of the gene expression of at least one gene selected from the group comprising ZFP64, DPP3, CCDC43, HIST2H4A/B, KDELC2 and MICALL1 in said sample,
c) repeating the above steps a and b at least once, preferably more than once, and
d) using said gene expressions determined in steps a) to c) to generate a time profile of said patient's response to said HDAC inhibitor treatment.

3. A method claim 1, which is method (I) or (II), wherein the gene expression and/or the change of the gene expression of said at least one gene is furthermore correlated with the probability of a positive outcome or with the probability of a negative outcome of the HDAC inhibitor treatment.

4. A method of claim 1, which is method (III), and is for stratification of a patient potentially in need of an HDAC inhibitor treatment comprising the following steps:
a) Providing a sample of said patient
b) Determining the gene expression of at least one gene selected from the group comprising ZFP64, DPP3, CCDC43, HIST2H4A/B, KDELC2 and MICALL1 in said sample
c) Correlating the determined gene expression of said at least one gene to the probability that an HDAC inhibitor treatment has a beneficial effect on said patient and
d) classifying said patient as responder or non-responder to said HDAC inhibitor treatment, based on the probability determined in step c.

5. The method according to claim 4, wherein said sample provided in step a) is provided before an HDAC inhibitor is administered to said patient,
wherein after step a) an HDAC inhibitor is added to said sample ex vivo to inhibit HDAC in said sample, and
wherein in step b) the gene expression of at least one gene is determined in said sample comprising said HDAC inhibitor.

6. A method of claim 1, which is method (IV), and is for predicting the probability of a positive outcome of an HDAC inhibitor treatment for a patient receiving said HDAC inhibitor treatment, the method comprising the following steps:
a) Providing a sample of said patient
b) Determining the gene expression of at least one gene selected from the group comprising ZFP64, DPP3, CCDC43, HIST2H4A/B, KDELC2 and MICALL1 in said sample,
c) Comparing said gene expression with the gene expression of said at least one gene in a sample provided from said patient prior to step a), and
d) Correlating the difference of the gene expression of said at least one gene in said sample provided in step a) and in said sample provided prior to step a) to the probability of a positive outcome of said HDAC inhibitor treatment for said patient.

7. A method according to claim 6, wherein said sample provided prior to step a) is provided from said patient before an HDAC inhibitor is administered to said patient, and
wherein said sample provided in step a) is provided after an HDAC inhibitor is administered to said patient, preferably after an HDAC inhibitor is administered to said patient for the first time.

8. A method of claim 1, which is method (V), and is for determining the gene expression of at least one gene as pharmacodynamic marker in a patient in need of an HDAC inhibitor treatment, the method comprising the following steps:
a) Providing a sample of said patient,
b) determining the gene expression and/or the ZFP64, DPP3, CCDC43, HIST2H4A/B, KDELC2 and MICALL1 in said sample
c) correlating the determined gene expression and/or the change of the gene expression of said at least one gene to the relative inhibition of HDAC by the HDAC inhibitor.

9. The method of claim 1, which is method (I), (II), (III), (IV) or (V), wherein the gene expression of said at least one gene is determined by measuring the level of at least one mRNA encoded by said at least one gene or a fragment thereof of at least 150 nucleotides in length, preferably at least 180 nucleotides in length, in said sample.

10. The method of claim 1, which is method (I), (II), (III), (IV) or (V), wherein the gene expression of said at least one gene is determined by measuring the level of at least one protein encoded by said at least one gene, or a domain of said protein, in said sample.

11. The method according to claim 10, wherein the level and/or the change of the level of said at least one protein or domain thereof is determined by the binding of an antibody or a probe comprising an antibody, wherein said antibody specifically binds to said at least one protein or domain thereof.

12. The method of claim 1, which is method (III), (IV) or (V), wherein the sample is taken either before starting of the HDAC inhibitor treatment or during HDAC inhibitor treatment.

13. The method of claim 1, which is method (I), (II), (III), (IV) or (V), wherein said sample is a sample of a bodily fluid, preferably a blood sample selected from the group comprising whole blood, serum or plasma, more preferably a peripheral blood sample selected from the group comprising whole blood, serum or plasma.

14. The method of claim 1, which is method (I), (II), (III), (IV) or (V), wherein the sample is a tissue sample, preferably a sample of diseased tissue, more preferably a biopsy from cancer tissue.

15. The method of claim 1, which is method (I), (II), (III), (IV) or (V), wherein steps a to c or a to b are repeated at least once, preferably more than once.

16. The method of claim 1, which is method (I), (II), (III), (IV) or (V), wherein the HDAC inhibitor is selected from the group comprising vorinostat, romidepsin, valproic acid, panobinostat, entinostat, belinostat, mocetinostat, givinostat and resminostat or a pharmaceutically acceptable salt thereof, preferably (E)-3-(1-(4-((dimethylamino)methyl)phenylsulfonyl)-1H-pyrrol-3-yl)-N-hydroxyacrylamide in free form or the hydrochloride or mesylate salt thereof.

17. A method
(1) for HDAC inhibitor treatment for a patient in need of said HDAC inhibitor treatment, comprising employing as a pharmacodynamic marker at least one gene or a protein encoded by said at least one gene, wherein said at least one gene is selected from the group comprising ZFP64, DPP3, CCDC43, HIST2H4A/B, KDELC2 and MICALL1 as a pharmacodynamic marker;
or
(2) for predicting the outcome of an HDAC inhibitor treatment for a patient in need of said HDAC inhibitor treatment, comprising employing at least one gene or a protein encoded by said at least one gene, wherein said at least one gene is selected from the group comprising ZFP64, DPP3, CCDC43, HIST2H4A/B, KDELC2 and MICALL1;
or
(3) for determining HDAC activity, comprising employing as a surrogate marker at least one gene or a protein encoded by said at least one gene, wherein said at least one gene is selected from the group comprising ZFP64, DPP3, CCDC43, HIST2H4A/B, KDELC2 and MICALL1:
or
(4) for stratifying a patient potentially in need of an HDAC inhibitor treatment as responder or non-responder, comprising employing at least one gene or a protein encoded by said at least one gene, wherein said at least one gene is selected from the group comprising ZFP64, DPP3, CCDC43, HIST2H4A/B, KDELC2 and MICALL1.

18. A method according to claim 17, which is method (2), and is for predicting the outcome of an HDAC inhibitor treatment for a patient in need of said HDAC inhibitor treatment, comprising employing at least one gene or a protein encoded by said at least one gene, wherein said at least one gene is selected from the group comprising ZFP64, DPP3, CCDC43, HIST2H4A/B, KDELC2 and MICALL1.

19. A method according to claim 17, which is method (3), and is for determining HDAC activity, comprising employing as a surrogate marker at least one gene or a protein encoded by said at least one gene, wherein said at least one gene is selected from the group comprising ZFP64, DPP3, CCDC43, HIST2H4A/B, KDELC2 and MICALL1.

20. A method according to claim 17, which is method (2), and is for stratifying a patient potentially in need of an HDAC inhibitor treatment as responder or non-responder, comprising employing at least one gene or a protein encoded by said at least one gene, wherein said at least one gene is selected from the group comprising ZFP64, DPP3, CCDC43, HIST2H4A/B, KDELC2 and MICALL1.

21. A kit
(A) for determining the gene expression of at least one gene selected from the group comprising ZFP64, DPP3, CCDC43, HIST2H4A/B, KDELC2 and MICALL1 in a sample, wherein the kit comprises probes which specifically bind to at least one mRNA encoded by said at least one gene or a fragment thereof of at least 150 nucleotides in length, preferably at least 180 nucleotides in length, and
wherein the kit optionally comprises one or more further components selected from the group comprising media, medium components, buffers, buffer components, RNA purification columns, DNA purification columns, dyes, nucleic acids including dNTP mix, enzymes including polymerases, and salts;
or
(B) for determining the level of at least one protein encoded by a gene selected from the group comprising ZFP64, DPP3, CCDC43, HIST2H4A/B, KDELC2 and MICALL1 in a sample:
wherein the kit comprises probes which specifically bind to at least one protein encoded by said at least one gene or a domain of said protein, and
wherein the kit optionally comprises one or more further components selected from the group comprising media, medium components, buffers, buffer components, membranes, ELISA plates enzyme substrates, dyes, enzymes including polymerases, and salts.

22. A kit according to claim 21, which is kit (B), and is for determining the level of at least one protein encoded by a gene selected from the group comprising ZFP64, DPP3, CCDC43, HIST2H4A/B, KDELC2 and MICALL1 in a sample:
wherein the kit comprises probes which specifically bind to at least one protein encoded by said at least one gene or a domain of said protein, and
wherein the kit optionally comprises one or more further components selected from the group comprising media, medium components, buffers, buffer components, membranes, ELISA plates enzyme substrates, dyes, enzymes including polymerases, and salts.

23. A method for determining the gene expression of at least one gene selected from the group comprising ZFP64, DPP3, CCDC43, HIST2H4A/B, KDELC2 and MICALL1 in a sample, which method is performed by a kit (A) or (B) according to claim 21.

24. A method according to claim 23, wherein said determined gene expression is correlated to HDAC activity in said sample.

25. A method according to claim 23, wherein said sample is provided from a patient potentially in need of an HDAC inhibitor treatment.

26. A method according to claim 1, which is method (I), (II), (III), (IV) or (V), and which is performed by a kit
(A) for determining the gene expression of at least one gene selected from the group comprising ZFP64, DPP3, CCDC43, HIST2H4A/B, KDELC2 and MICALL1 in a sample, wherein the kit comprises probes which specifically bind to at least one mRNA encoded by said at least one gene or a fragment thereof of at least 150 nucleotides in length, preferably at least 180 nucleotides in length, and
wherein the kit optionally comprises one or more further components selected from the group comprising media, medium components, buffers, buffer components, RNA purification columns, DNA purification columns, dyes, nucleic acids including dNTP mix, enzymes including polymerases, and salts;
or
(B) for determining the level of at least one protein encoded by a gene selected from the group comprising ZFP64, DPP3, CCDC43, HIST2H4A/B, KDELC2 and MICALL1 in a sample:
wherein the kit comprises probes which specifically bind to at least one protein encoded by said at least one gene or a domain of said protein, and
wherein the kit optionally comprises one or more further components selected from the group comprising media, medium components, buffers, buffer components, membranes, ELISA plates enzyme substrates, dyes, enzymes including polymerases, and salts.

27. (canceled)

28. A method of treating a patient potentially in need of an HDAC inhibitor treatment, the method comprising administering to the patient an HDAC inhibitor, wherein before and/or during said method at least one gene selected from the group comprising ZFP64, DPP3, CCDC43, HIST2H4A/B, KDELC2 and MICALL1, at least one mRNA corresponding to said at least one gene, or at least one protein encoded by said at least one gene is used for determining the probability of an effect of the HDAC inhibitor treatment to said patient, or for determining whether said patient is a responder to the HDAC inhibitor treatment.

29. The method according to claim 28 wherein the HDAC inhibitor is selected from the group comprising vorinostat, romidepsin, valproic acid, panobinostat, entinostat, belinostat, mocetinostat, givinostat and resminostat or a pharmaceutically acceptable salt thereof, preferably (E)-3-(1-(4-((dimethylamino)methyl)phenylsulfonyl)-1H-pyrrol-3-yl)-N-hydroxyacrylamide in free form or a hydrochloride salt or a mesylate salt thereof.

30. A method of claim 1, which is method (I), and is for determining an effect of an HDAC inhibitor treatment, the method comprising the following steps:
a) Providing a sample of a patient receiving said HDAC inhibitor treatment,
b) determining the gene expression and/or the change of the gene expression of at least one gene selected from the group comprising ZFP64, DPP3, CCDC43, HIST2H4A/B, KDELC2 and MICALL1 in said sample,
c) correlating the determined gene expression and/or the change of the gene expression of said at least one gene to an effect of said HDAC inhibitor treatment in said patient.

31. A kit according to claim 21, which is kit (A), and is for determining the gene expression of at least one gene selected from the group comprising ZFP64, DPP3, CCDC43, HIST2H4A/B, KDELC2 and MICALL1 in a sample, wherein the kit comprises probes which specifically bind to at least one mRNA encoded by said at least one gene or a fragment thereof of at least 150 nucleotides in length, preferably at least 180 nucleotides in length, and
wherein the kit optionally comprises one or more further components selected from the group comprising media, medium components, buffers, buffer components, RNA purification columns, DNA purification columns, dyes, nucleic acids including dNTP mix, enzymes including polymerases, and salts.

32. A method according to claim 17, which is method (1), and is for HDAC inhibitor treatment for a patient in need of said HDAC inhibitor treatment, comprising employing as a pharmacodynamic marker at least one gene or a protein encoded by said at least one gene, wherein said at least one gene is selected from the group comprising ZFP64, DPP3, CCDC43, HIST2H4A/B, KDELC2 and MICALL1 as a pharmacodynamic marker.


SEQUENCE LISTING (12 sequences)

SEQ ID NO: 1 - DNA, 73 bp, Artificial Sequence
  Description of Artificial Sequence Synthetic ZFP64 target oligonucleotide
cacctcggagacccagacaatcacagtttcagctccagaatttgtttttgaacatggctatcaaacttacctg

SEQ ID NO: 2 - DNA, 20 bp, Artificial Sequence
  Description of Artificial Sequence Synthetic forward primer
cacctcggagacccagacaa

SEQ ID NO: 3 - DNA, 25 bp, Artificial Sequence
  Description of Artificial Sequence Synthetic reverse primer
caggtaagtttgatagccatgttca

SEQ ID NO: 4 - DNA, 19 bp, Artificial Sequence
  Description of Artificial Sequence Synthetic forward primer
cacctcggagacccagaca

SEQ ID NO: 5 - DNA, 24 bp, Artificial Sequence
  Description of Artificial Sequence Synthetic reverse primer
caggtaagtttgatagccatgttc

SEQ ID NO: 6 - DNA, 20 bp, Artificial Sequence
  Description of Artificial Sequence Synthetic reversed transcription forward primer 1
acctgcccacggaaagtaat

SEQ ID NO: 7 - DNA, 20 bp, Artificial Sequence
  Description of Artificial Sequence Synthetic reversed transcription reverse primer 1
tatggggtttgtctcccgtg

SEQ ID NO: 8 - DNA, 20 bp, Artificial Sequence
  Description of Artificial Sequence Synthetic reversed transcription forward primer 2
acccagacaatcacaggttg

SEQ ID NO: 9 - DNA, 21 bp, Artificial Sequence
  Description of Artificial Sequence Synthetic reversed transcription reverse primer 2
gctaaagcacttgccacagac

SEQ ID NO: 10 - protein, 49 aa, Artificial Sequence
  Description of Artificial Sequence Synthetic Abcam ab66658 - rabbit polyclonal ZFP64 antibody immunogen polypeptide
DGGQNIAVATTAPPVFSSSSQQELPKQTYSIIQGAAHPALLCPADSIPD

SEQ ID NO: 11 - protein, 92 aa, Artificial Sequence
  Description of Artificial Sequence Synthetic immunogen polypeptide
SFDTKQPSNLSKHMKKFHGDMVKTEALERKDTGRQSSRQVAKLDAKKSFHCDICDASFMREDSLRSHKRQHSEYSESKNSDVTVLQFQIEPS

SEQ ID NO: 12 - protein, 4 aa, Homo sapiens
KDEL